(19) United States
(12) Patent Application Publication
(10) Pub. No.: US 2012/0263724 A1
(43) Pub. Date: Oct. 18, 2012

(54) Escherichia coli o157:h7 proteins and uses thereof

(76) Inventors: Manohar John, Ames, IA (US); Indira T. Kudva, Ames, IA (US); Stephen B. Calderwood, Wellesley, MA (US); Bryan Krastins, Somerville, MA (US); David A. Sarracino, Andover, MA (US)

(21) Appl. No.: 11/919,021

(22) PCT Filed: Apr. 20, 2006
(86) PCT No.: PCT/US06/15100

§ 371 Date: Jun. 14, 2010

Related U.S. Application Data
(60) Provisional application No. 60/673,599, filed on Apr. 20, 2005.

(51) Int. Cl. C07K 14/245; C12N 15/63; C12N 1/21; C12N 1/19; C12N 5/10; C12P 21/00; C12P 21/08; C12Q 1/68; G01N 33/566; A61K 39/108; A61P 37/04; A61P 31/04; C12N 15/31; C07K 16/12; A61K 39/40; C40B 20/08

(52) U.S. Cl. 424/139.1; 506/6; 435/320.1; 435/252.33; 435/254.21; 435/348; 435/325; 435/357; 435/367; 435/365; 435/70.2; 435/70.21; 435/6.13; 436/501; 530/350; 424/190.1; 536/23.7; 530/387.9

(57) ABSTRACT

Immunogenic Escherichia coli O157:H7 (0157) proteins expressed during infection of a mammal, are described. In particular, 0157 proteins expressed specifically in vivo during human and cattle infection were identified. These proteins, mapped to the backbone, O-islands (OIs) and pO157. Because these-proteins are expressed during infection, and might help pathogens adapt to and counter hostile in vivo environments, those proteins identified in this study are useful as targets for drug and vaccine development. Also, such proteins are useful as markers of 0157 infection in stool specimens. Also described are methods of identifying immunogenic proteins.

### BACKGROUND OF THE INVENTION

The invention relates to immunogenic Escherichia coli O157:H7 (O157) proteins, in particular, proteins that are immunogenic in humans or cattle or both. This invention also relates to methods for identifying immunogenic proteins derived from a pathogen.

Enterohemorrhagic Escherichia coli (EHEC) O157:H7 (O157) is a pathogen which causes disease ranging from acute, self-resolving watery diarrhea to hemorrhagic colitis (HC) and the potentially fatal hemolytic uremic syndrome (HUS). Currently, no therapies are available to lessen the potential morbidity and mortality of this infection.

A common route for human infection is the consumption of beef contaminated with EHEC. Cattle feces seeping into water and the slaughter of infected animals are considered to be the primary sources of E. coli O157 infection in humans. An E. coli O157 vaccine for cattle is a potential solution for preventing human disease because it deals with the problem at the source (the cattle's gastrointestinal tract) and prevents the bacteria from getting into the water supply and food chain, where it can cause illness in humans.

O157 is thought to have evolved from a strain of the enteropathogenic E. coli (EPEC) O55:H7 bearing the pathogenicity island termed the locus for enterocyte effacement (LEE), through the acquisition of bacteriophages encoding Shiga toxins type 1 (stx₁) and/or 2 (stx₂), a virulence plasmid (pO157), transition of somatic antigen O55 to O157 and loss of sorbitol fermentation and β-glucuronidase activity (Kaper et al. 2004. Nat Rev Microbiol 2:123-140). HUS as a complication of O157 infection has been associated with the presence of the stx₂ or its variant stx_{2c} genes in the infecting O157 strain (Ibid). In addition, the characteristic attaching and effacing (A/E) lesions produced by this organism on the human colonic epithelium are a result of proteins encoded on the LEE, including the adhesion molecule intimin-γ (Eae), its receptor (Tir), the type III protein secretion system that secretes a variety of LEE-encoded translocator proteins (EspA, EspB, and EspD) that translocate effectors into host cells, and effector proteins (Tir, EspG, EspF, Map, and EspH) that modulate the host cell cytoskeleton (Ibid). The type III secretion system translocates Tir into the host cell, with subsequent trafficking to the host cell membrane. Intimin binding of Tir leads to host cell actin rearrangement and formation of A/E lesions. Other putative virulence factors are encoded on pO157 and include an enterohemolysin (Ehx), an immunomodulator (Lif), and a serine protease (EspP) (Ibid). Hence several factors may be involved in E. coli O157 pathogenesis and research is ongoing to understand the complexity of this infection.

The sequenced O157 EDL933 genome shows that although this organism shares 4.1 Mb of DNA with E. coli K-12 (termed backbone), it has 1.34 Mb of DNA distributed among 177 DNA segments termed O-islands that is absent in K-12 (Perna et al. 2001. Nature 409:529-533). Of the genes found in these O-islands, only 40% have been assigned a function and several remain to be characterized (Ibid). Collective evidence indicates that intimin-γ and the Shiga toxins act in concert with other unidentified virulence factors, encoded by both the O-island and backbone sequences, to cause the spectrum of O157 disease (Kaper et al. 2004. Nat Rev Microbiol 2:123-140; Torres and Kaper 2003. Infect Immun 71:4985-4995).

### SUMMARY OF THE INVENTION

Using in vivo-induced antigen technology (IVIAT), a modified immunoscreening technique that circumvents the need for animal models, we have directly identified immunogenic Escherichia coli O157:H7 (O157) proteins expressed specifically during human infection, but not during growth under standard laboratory conditions. IVIAT identified 223 O157 proteins expressed specifically in vivo during human infection, several of which were unique to this study. These in vivo-induced (ivi) proteins, encoded by ivi-genes, mapped to the backbone, O-islands (OIs) and pO157. Lack of in vitro expression of O157-specific ivi-proteins was confirmed by proteomic analysis of a mid-exponential phase culture of O157 grown in LB broth. Because ivi-proteins are expressed in response to specific cues during infection, and might help pathogens adapt to and counter hostile in vivo environments, those identified in this study are useful as targets for drug and vaccine development. Also, such proteins may be exploited as markers of O157 infection in stool specimens.

In addition to the IVIAT system, we developed a novel application of proteomics, Proteomics-based Expression Library Screening (PELS), to independently identify 207 proteins which are immunogenic in cattle. These only include proteins produced during infection of cattle, and not those produced exclusively in vitro. As with the proteins identified by IVIAT, these proteins are useful targets for drug and vaccine development, and as diagnostic markers for O157 infection.

In one aspect, the invention features an isolated nucleic acid molecule including a sequence substantially identical to any one of polynucleotides described in Tables 2, 3, or 7. In one embodiment, the isolated nucleic acid molecule includes any of the above-described sequences or a fragment thereof; and is derived from a pathogen (e.g., from a bacterial pathogen such as E. coli O157). Additionally, the invention includes a vector and a cell, each of which includes at least one of the isolated nucleic acid molecules of the invention; and a method of producing a recombinant polypeptide involving providing a cell transformed with a nucleic acid molecule of the invention positioned for expression in the cell, culturing the transformed cell under conditions for expressing the nucleic acid molecule, and isolating a recombinant polypeptide. The invention further features recombinant polypeptides produced by such expression of an isolated nucleic acid molecule of the invention, and substantially pure antibodies that specifically recognize and bind such recombinant polypeptides.

In an another aspect, the invention features a substantially pure polypeptide including an amino acid sequence that is substantially identical to the amino acid sequence of any one of the amino acids described in Tables 2, 3, or 7. In one embodiment, the substantially pure polypeptide includes any of the above-described sequences or an immunogenic fragment thereof; and is derived from a pathogen (e.g., from a bacterial pathogen such as E. coli O157).

In yet another related aspect, the invention features a method for identifying a compound which is capable of decreasing the expression of a pathogenic factor (e.g., at the transcriptional or post-transcriptional levels), involving (a) providing a pathogenic cell expressing any one of the isolated nucleic acid molecules of the invention; and (b) contacting the pathogenic cell with a candidate compound, a decrease in expression of the nucleic acid molecule following contact with the candidate compound identifying a compound which decreases the expression of a pathogenic virulence factor. In preferred embodiments, the pathogenic cell infects a mammal (e.g., a human).

In yet another related aspect, the invention features a method for identifying a compound which binds a polypeptide, involving (a) contacting a candidate compound with a substantially pure polypeptide including any one of the amino acid sequences of the invention under conditions that allow binding; and (b) detecting binding of the candidate compound to the polypeptide.

In addition, the invention features a method of treating a pathogenic infection in a mammal, involving (a) identifying a mammal having a pathogenic infection; and (b) administering to the mammal a therapeutically effective amount of a composition which inhibits the expression or activity of a polypeptide encoded by any one of the nucleic acid molecules of the invention. In preferred embodiments, the pathogen is E. coli O157.

In yet another aspect, the invention features a method of treating a pathogenic infection in a mammal, involving (a) identifying a mammal having a pathogenic infection; and (b) administering to the mammal a therapeutically effective amount of a composition which binds and inhibits a polypeptide encoded by any one of the amino acid sequences of the invention. In preferred embodiments, the pathogenic infection is caused by E. coli O157.

In general, the invention includes any nucleic acid sequence which may be isolated as described herein or which is readily isolated by homology screening or polymerase chain reaction (PCR) amplification using the nucleic acid sequences of the invention. Also included in the invention are polypeptides which are modified in ways which do not abolish their pathogenic activity (assayed, for example as described herein). Such changes may include certain mutations, deletions, insertions, or post-translational modifications, or may involve the inclusion of any of the polypeptides of the invention as one component of a larger fusion protein. Also, included in the invention are polypeptides that have lost their pathogenicity.

Thus, in other embodiments, the invention includes any protein that is substantially identical to a polypeptide of the invention. Such homologs include other substantially pure naturally-occurring polypeptides as well as allelic variants; proteins having substantial identity to any protein found in Tables 2, 3, or 7 isolated from naturally occurring related strains of E. coli, or other pathogenic bacteria; natural mutants; induced mutants; proteins encoded by DNA that hybridizes to any one of the nucleic acid sequences of the invention under high stringency conditions or, less preferably, under low stringency conditions (e.g., washing at 2×SSC at 40° C. with a probe length of at least 40 nucleotides); and proteins specifically bound by antisera of the invention.

The invention further includes analogs of any naturally-occurring polypeptide of the invention. Analogs can differ from the naturally-occurring the polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino acid sequence of the invention. The length of sequence comparison is at least 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis according to standard methods. Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the invention also includes fragments of any one of the polypeptides of the invention. As used herein, the term “fragment,” means at least 5, preferably at least 20 contiguous amino acids, preferably at least 30 contiguous amino acids, more preferably at least 50 contiguous amino acids, more preferably at least 80 or more contiguous amino acids, and most preferably at least 200 amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Furthermore, the invention includes nucleotide sequences that facilitate specific detection of any of the nucleic acid sequences of the invention. Thus, for example, nucleic acid sequences described herein or fragments thereof may be used as probes to hybridize to nucleotide sequences by standard hybridization techniques under conventional conditions. Sequences that hybridize to a nucleic acid sequence coding sequence or its complement are considered useful in the invention. Sequences that hybridize to a coding sequence of a nucleic acid sequence of the invention or its complement and that encode a polypeptide of the invention are also considered useful in the invention. As used herein, the term “fragment,” as applied to nucleic acid sequences, means at least 5 contiguous nucleotides, preferably at least 10 contiguous nucleotides, more preferably at least 20 to 30 contiguous nucleotides, more preferably at least 40 to 80, more preferably at least 100 to 150, more preferably at least 200 to 300, or most preferable at least 400 to 600 or more contiguous nucleotides. Fragments of nucleic acid sequences can be generated by methods known to those skilled in the art.

The invention further provides a method for inducing an immunological response in a subject, particularly a human or cattle, which includes inoculating the subject with, for example, any of the polypeptides (or a fragment or analog thereof or fusion protein) of the invention to produce an antibody and/or a T cell immune response to protect the subject from infection, especially bacterial infection (e.g., an E. coli infection). The invention further includes a method of inducing an immunological response in a subject which includes delivering to the subject a nucleic acid vector to direct the expression of a polypeptide described herein (or a fragment or fusion thereof) in order to induce an immunological response.

The invention also includes vaccine compositions including the polypeptides or nucleic acid sequences of the invention. For example, the polypeptides of the invention may be used as an antigen for vaccination of a subject to produce specific antibodies which protect against an E. coli infection. The invention therefore includes a vaccine formulation which includes an immunogenic recombinant polypeptide of the invention together with a suitable carrier.

In another embodiment, peptide vaccines can be utilized as a prophylactic or therapeutic vaccine in a number of ways, including: 1) as monomers or multimers, 2) combined contiguously or non-contiguously with additional sequences that may facilitate aggregation, promote presentation or processing of the epitope (e.g., class targeting sequences) and/or additional antibody, T helper or CTL epitopes to increase the immunogenicity of the peptide as a means to enhance efficacy of the vaccine, 3) chemically modified or conjugated to agents that would increase the immunogenicity or delivery of the vaccine (e.g., fatty acid or acyl chains, KLH, tetanus toxoid, or cholera toxin), 4) any combination of the above, 5) any of the above in combination with adjuvants, including but not limited to inorganic gels such as aluminium hydroxide, and water-in-oil emulsions such as incomplete Freund's adjuvant, aluminum salts, saponins or triterpenes, MPL, cholera toxin, ISCOM'S®, PROVAX®, DETOX®, SAF, Freund's adjuvant, Alum®, Saponin®, among others.

An important aspect of this invention is that it includes vaccines formulated for both humans and cattle which include any of the proteins or fragments thereof described herein. The invention also provides for the production of antibodies (e.g., polyclonal or monoclonal) directed to any of the proteins or immunogenic fragments thereof described herein. Such antibodies are especially useful for passive immunization.

The invention further provides compositions (e.g., nucleotide sequence probes), polypeptides, antibodies, and methods for the diagnosis of a pathogenic condition.

By “isolated nucleic acid molecule” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “polypeptide” or “protein” is meant any chain of amino acids, regardless of length or post-translational modification (for example, glycosylation or phosphorylation).

By a “substantially pure polypeptide” is meant a polypeptide of the invention that has been separated from components which naturally accompany it. Typically, the polypeptide is substantially pure when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. A substantially pure polypeptide of the invention may be obtained, for example, by extraction from a natural source (for example, a pathogen); by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “substantially identical” is meant a polypeptide or nucleic acid molecule exhibiting at least 25% identity to a reference amino acid sequence (for example, any one of the amino acid sequences described herein) or nucleic acid sequence (for example, any one of the nucleic acid sequences described herein). Preferably, such a sequence is at least 30%, 40%, 50%, 60%, more preferably 80%, and most preferably 90% or even 95%, 96%, 97%, 98% or 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transformed cell” is meant a cell into which (or into an ancestor of which) has been introduced, by means of recombinant DNA techniques, a DNA molecule encoding (as used herein) a polypeptide of the invention. Exemplary polypeptides include those identified in Tables 2, 3, 7, and the amino acid sequences set forth in SEQ ID NOs: 1-340.

By “positioned for expression” is meant that the DNA molecule is positioned adjacent to a DNA sequence which directs transcription and translation of the sequence (i.e., facilitates the production of, for example, a recombinant polypeptide of the invention, or an RNA molecule).

By “purified antibody” is meant antibody which is at least 60%, by weight, free from proteins and naturally-occurring organic molecules with which it is naturally associated. Preferably, the preparation is at least 75%, more preferably 90%, and most preferably at least 99%, by weight, antibody. A purified antibody of the invention may be obtained, for example, by affinity chromatography using a recombinantly-produced polypeptide of the invention and standard techniques.

By “specifically binds” is meant a compound or antibody which recognizes and binds a polypeptide of the invention but which does not substantially recognize and bind other molecules in a sample, for example, a biological sample, which naturally includes a polypeptide of the invention.

By “derived from” is meant isolated from or having the sequence of a naturally-occurring sequence (e.g., a cDNA, genomic DNA, synthetic, or combination thereof).

By “infected” or “infection” is meant a state produced by the establishment of an infective agent in or on a subject; including a disease resulting from an infection. These terms also include an act or process of infecting as well as the establishment of a pathogen in a subject after invasion.

By “inhibiting a pathogen” is meant the ability of a candidate compound to decrease, suppress, attenuate, diminish, or arrest the development or progression of a pathogen-mediated disease or an infection in a eukaryotic subject. Preferably, such inhibition decreases pathogenicity by at least 5%, more preferably by at least 25%, and most preferably by at least 50%, as compared to symptoms in the absence of the candidate compound in any appropriate pathogenicity assay (for example, those assays described herein). In one particular example, inhibition may be measured by monitoring pathogenic symptoms in a subject exposed to a candidate compound or extract, a decrease in the level of symptoms relative to the level of pathogenic symptoms in a subject not exposed to the compound indicating compound-mediated inhibition of the pathogen.

By “pathogenic virulence factor” is meant a cellular component (e.g., a protein such as a transcription factor, as well as the gene which encodes such a protein) without which the pathogen is incapable of causing disease or infection in a eukaryotic subject. Exemplary virulence factors are those described herein which are expressed specifically during human infection.

By “immune response” is meant a series of molecular, cellular, and organismal events that are induced when an antigen is encountered by the immune system. These may include the expansion of B- and T-cells and the production of antibodies. Aspects of an immune response, such as the expansion of T cell, B cell, or other antigen presenting cell populations may take place in vitro for administration to a subject. The immune response may provide a defense against foreign substances or organisms. To determine whether an immune response has occurred and to follow its course, the immunized subject can be monitored for the appearance of immune reactants directed at the specific antigen.

By “immunogenic” is meant the ability of a substance or composition or pathogen to induce an immune response.

By “adjuvant” is meant a compound (e.g., an immunomodulator) which non-specifically stimulates or enhances an immune response (e.g., production of IgA antibodies). Administration of an adjuvant in conjunction with an immunogenic composition facilitates the induction of an immune response to the immunogenic compound and assists in the prevention, amelioration, or cure of a pathogenic infection or disease.

By “treating” or “treatment” is meant reduction of the severity, progression, spread, and/or frequency of symptoms, elimination of symptoms and/or underlying cause, prevention of the occurrence of symptoms and/or their underlying cause, and improvement or remediation of damage. Exemplary treatment modalities include therapeutic treatment as well as prophylactic, or suppressive measures for an infection.

By “prophalactic” is meant a treatment that results in the prevention of disease in a subject.

By “therapeutic” is meant a treatment that results in alleviation or amelioration from a pre-existing disease.

A feature of the invention is the rapidity of proteome-wide immunogenic protein-identification, which renders Proteomics-based Expression Library Screening (herein referred to as “PELS”) an ideal alternative/complement to emerging protein chip/array technologies. Other attractive features include broad applicability, robustness, and an elimination of subjective bias. Also, because proteins are expressed from genes on inserts within clones of a genomic DNA expression library, a more comprehensive determination of the immunoproteome of the cognate pathogen is possible.

The invention provides a number of targets that are useful for the development of drugs that specifically block the pathogenicity of a microbe. In addition, the methods of the invention provide a facile means to identify compounds that are safe for use in eukaryotic subjects (i.e., compounds which do not adversely affect the normal development and physiology of the organism), and efficacious against pathogenic microbes (i.e., by suppressing the virulence of a pathogen). In addition, the methods of the invention provide a route for analyzing virtually any number of compounds for an anti-virulence effect with high-volume throughput, high sensitivity, and low complexity. The methods are also relatively inexpensive to perform and enable the analysis of small quantities of active substances found in either purified or crude extract form.

Other features and advantages of the invention will be apparent from the detailed description, and from the claims.

### BRIEF DESCRIPTION OF THE DRAWINGS

FIG. 1. Reactivity of pooled, unadsorbed (Panel A) and adsorbed (Panel B) HUS sera against two known O157 virulence proteins, intimin and EspA, and an unrelated negative control protein, PilA, from V. cholerae.

FIG. 2. EIA reactivity of sera with in vitro grown O157 lysates after each step in sequential adsorption. OD₄₀₅ values were corrected for background and for dilution during adsorption.

FIG. 3. Graphical representation of the location of ivi-genes on the chromosome of E. coli O157 strain EDL 933. Outer and inner circles show the positions of ivi-genes on the backbone and O-islands, respectively. The black numbers on the outside of the inner circle refer to individual OIs (Perna et al. 2001. Nature 409:529-533). Individual OI-genes, phage-associated genes, the LEE (encoding intimin-γ), CP-933V (encoding Stx1), and BP-933W (encoding Stx2) are also indicated. OI groups, I, II and III are indicated by brackets (see text). The innermost circle shows the scale in base pairs. Figure was created using the Genvision software from DNASTAR.

FIG. 4. Graphical representation of the location of ivi-genes on the pO157 plasmid of E. coli O157 strain EDL 933. Outer circle shows the position of ivi-genes (magenta), and the inner circle, the scale in base pairs. Figure was created using the Genvision software from DNASTAR.

FIG. 5. Flow chart of PELS screening process.

FIG. 6. SDS-PAGE profiles of recombinant O157 proteins in elutions from columns coupled to bait PAbs (“charged”) or “uncharged” columns. Multiple O157 expression library cultures were induced with a range of IPTG concentrations at an initial OD₆₀₀ of 0.6. Following incubations at different temperatures for varying lengths of time, cell-lysates and pellets prepared from pooled, pelleted cultures were loaded on “charged” or “uncharged” columns. Elutions were fractionated on SDS-PAGE Tris-Glycine 8-16% gradient gels and visualized by SimplyBlue Safe staining. FIG. 6A shows elutions from a “charged” column (test) loaded with lysate fractions. FIG. 6B shows elutions from an “uncharged” column (control) loaded with lysate fractions. FIG. 6C shows elutions from a “charged” column (test) loaded with pellet fractions. FIG. 6D shows elutions from an “uncharged” column (control) loaded with pellet fractions. (1) indicates the molecular weight ladder.

FIG. 7. Reactivity of purified E. coli O157 LPS and recombinant E. coli BL21(DE3) (pET-30b) expressing various bacterial proteins with pooled hyperimmune sera from cattle experimentally infected with E. coli O157. FIG. 7A is a dot immunoblot of purified O157 LPS, spotted in duplicate, screened with pooled, unadsorbed hyperimmune bovine sera. FIG. 7B is a Dot immunoblot of O157 LPS, spotted in duplicate (test); 2, Colony immunoblot of four recombinant clones expressing PilA of V. cholerae, an irrelevant protein (control); and 3, Colony immunoblot of four recombinant clones expressing EspB of E. coli O157 (test), screened with pooled hyperimmune bovine sera adsorbed against purified E. coli O157 LPS.

### DETAILED DESCRIPTION

A pathogen expresses specific proteins during infection of a subject. The methods described herein identify such proteins. In the first two working examples, immunogenic E. coli proteins are identified that are expressed during infection of humans and cattle. An additional example is then presented which provides methods useful to identify immunogenic proteins from virtually any pathogen.

### Example 1

### E. coli Antigens Identified in Humans

To date, the main impediment to identifying a broader complement of virulence factors in this pathogen has been the lack of an animal model that mimics the spectrum of human disease. Also, the potentially fatal sequelae that can follow O157 infection preclude human volunteer studies. We circumvented these limitations and exploited the human immune response following O157 infection itself to identify a panel of microbial factors that might contribute to the pathogenicity of this organism. In particular, we used a modified immunoscreening technique called in vivo-induced antigen technology (IVIAT) (Handfield et al. 2000. Trends Microbiol 8:336-339) to identify immunogenic O157 proteins that are either expressed during infection but not during growth in standard laboratory media, or expressed at significantly higher levels in vivo than in vitro. The rationale was that such antigens expressed in response to unique signals encountered within the gastrointestinal tract might contribute to pathogen adaptation and survival within the gut, and hence play important roles in the virulence of this organism. Below we describe the identification of O157 proteins that are expressed uniquely during human infection but not during in vitro growth, and indicate that several of the genes encoding such proteins are conserved through evolution. These identified proteins are useful as targets for development of diagnostics, drugs, and vaccines.

### Materials and Methods

Recombinant DNA Methods.

Isolation of plasmid DNA, restriction digestions, and agarose gel electrophoresis were performed using standard procedures (Sambrookand Russel 2001. A Laboratory Manual. Cold Spring Harbor Laboratory Press, Cold Spring Harbor, N.Y.). All enzymes for restriction digestions, DNA modifications and ligations were from New England Biolabs, Beverly, Mass. DNA sequencing was performed at the DNA Sequencing Core Facility, Department of Molecular Biology, Massachusetts General Hospital, using ABI Prism DiTerminator cycle sequencing with AmpliTaq DNA polymerase FS with an ABI 377 DNA sequencer (Perkin-Elmer Applied Biosystems Division, Foster City, Calif.). Oligonucleotides for PCR and sequencing were obtained from the DNA Synthesis Core Facility, Department of Molecular Biology, Massachusetts General Hospital. Plasmids were electroporated into E. coli DH5α or BL21(DE3) using a Gene Pulser (Bio-Rad Laboratories, Richmond, Calif.) as instructed by the manufacturer. Electroporation conditions were 2,500 V at 25-mF capacitance, producing time constants of 4.8 to 4.9 ms.

Bacterial Strains, Plasmids, and Growth Conditions.

An isolate of O157, from a patient who recovered from recent, clinically diagnosed HUS, and contributed a serum sample to the pool of convalescent-phase sera for probing the expression library, was used to construct the expression DNA library (see below). E. coli X21-Blue (pEB313) expressed an intracellular derivative of intimim-γ, His₆-intimin-γ (McKee et al. 1996. Infect Immun 64:2225-2233), from which the putative signal sequence of 34 amino acids had been removed (gift of Dr. Alison O'Brien, Uniformed Services University of the Health Sciences, Bethesda, Md.). E. coli DH5a (pCVD468, pREP4) expressed a genetically engineered version of EspA, His₆-EspA (Karpman et al. 2002. Pediatr Nephrol 17:201-211), which was a gift from Dr. James B. Kaper, University of Maryland School of Medicine, Baltimore, Md. Bacterial strains were grown in vitro in Luria-Bertani (LB) medium, and maintained at −70° C. in LB broth containing 15% glycerol. Kanamycin (kan) and ampicillin (amp) were used at concentrations of 50 μg/ml and 100 μg/ml, respectively.

Patient and Control Sera.

Convalescent-phase sera (approximately 500 μl/patient) were obtained from four patients who had recovered from HUS following O157 infection (HUS-convalescent sera). Age of the patients ranged from 2-10 yrs, and sera were collected on day 13-96 post-illness. A serum sample from a healthy pediatric patient was used as the control. All of the above serum samples were collected at the Children's Hospital and Regional Medical Center, Seattle, Wash., for routine laboratory investigations and only excess sera was used for this study. The Institutional Review Board of the Children's Hospital and Regional Medical Center approved the use of these sera, which were stored at −70° C. until used.

Assessment of Reactivity of Pooled, Unadsorbed HUS-Convalescent and Healthy Control Sera with Immunogenic O157 Proteins.

Sera were assessed by examining their reactivity via colony immunoblotting (described below), against E. coli XL1-Blue (pEB313) expressing His₆-intimin-γ, plated on LB-amp plates (McKee et al. 1996. Infect Immun 64:2225-2233), and E. coli DH5α (pCVD468, pREP4) expressing His₆-EspA, on LB-amp+kan plates (Karpman et al. 2002. Pediatr Nephrol 17:201-211). Both EspA and intimin-γ are expressed during human infection and targeted by the immune response (Ibid; McKee et al. 1996. Infect Immun 64:2225-2233).

Adsorption of HUS-Convalescent and Control Sera.

To compensate for variations in immune responses of individual patients and identify the widest array of O157 antigens, equal volumes of HUS-convalescent serum samples from four patients were pooled, and sequentially adsorbed against the O157 isolated from one of the four patients (the same O157 isolate used to generate the expression library).

The adsorption protocol has been described previously (Hang et al. 2003. Proc Nat Acad Sci USA 100:8508-8513). Briefly, a protease inhibitor cocktail formulated for bacterial cells and containing 4-(2-aminoethyl)benzenesulfonyl fluoride (AEBSF; 23 mM), ethylenediaminetetraacetic acid (EDTA; 100 mM), bestatin (2 mM), pepstatin A (0.3 mM) and E-64 (0.3 mM), was prepared per the manufacturer's (Sigma, St. Louis, Mo.) instructions, and then added to intact cells and cell-lysates at a dilution of 1:10. Pooled, BUS-convalescent sera were sequentially adsorbed against in vitro grown (LB broth, 37° C.) O157 whole cells, cell-lysates (prepared by 3 cycles of freezing and thawing, followed by sonication) and heat-denatured cell-lysates (Ibid). Adsorbed sera were stored at −70° C. until further use.

Individual HUS-convalescent serum samples from each of the four patients, and the control serum sample, were adsorbed against whole cells, cell-lysates and heat-denatured cell-lysates of in vitro grown (LB-kan broth, 37° C.) expression host, E. coli BL21(DE3) containing the native expression plasmids, pET-30 abc, in a similar manner.

The efficiency of adsorption of pooled, HUS-convalescent sera was assessed using an enzyme immunoassay described previously (Ibid) and detailed below. Adsorption efficiency was further evaluated by reacting sera with recombinant clones expressing His₆-intimin-γ and His₆-EspA, via colony immunoblotting as described below.

Efficiency of Adsorption of Pooled, HUS-Convalescent Sera.

Microtiter wells were coated with 100 μl of a 1:2 dilution of in vitro grown O157 lysate (same isolate used to make the DNA expression library) in 50 mM carbonate buffer (pH 9.6), prepared by three cycles of freezing and thawing followed by sonication. Following overnight incubation at room temperature, wells were washed with phosphate buffered saline (PBS) containing 0.05% of Tween 20 (PBS-T), and blocked with a 1% solution of BSA. After 1 h incubation at 37° C., wells were emptied, and 100 μl dilutions (1:200 to 1:25,600) of sera, removed from the pool after each adsorption step, were added to wells. The wells were incubated for 1 h at 37° C. and washed, after which 100 μl of a 1:1000 dilution of horseradish peroxidase-conjugated goat anti-human affinity-purified IgG reactive against all classes of human immunoglobulins (ICN, Cappel, Aurora, Ohio), were added to the wells. Wells were incubated for 1 h at 37° C. and washed with PBS-T. Reactions were developed with a 1 mg/ml solution of 2,2′-azinobis(ethylbenzthiazolinesulphonic acid) (ABTS; Sigma, St. Louis, Mo.) with 0.1% H₂O₂ (Sigma). The OD₄₀₅ was determined kinetically with a Vmax microplate reader (Molecular Devices Corporation, Sunnyvale, Calif.). Plates were read for 5 min at 19-s intervals, and the maximum slope for an OD change of 0.2 U was determined as milli-OD units/min (John et al. 2000. Infect Immun 68:1171-1175).

Construction of an Inducible, E. Coli O157:H7 Genomic DNA Expression Library.

Polymorphic amplified typing sequences (PATS), a powerful and user-friendly typing methodology for bacterial pathogens that compares well with pulsed-field gel electrophoresis (PFGE) (Kudva et al. 2002. J Clin Microbiol 40:1152-1159; Kudva et al. 2004. J Clin Microbiol 42:2388-2397), profiled the cognate O157 isolates from the 4 HUS patients as heterogeneous. We therefore selected one isolate at random, purified genomic DNA and generated the DNA expression library.

To generate the expression library, vector DNA was prepared by digesting with the restriction enzyme BamHI (New England Biolabs, Beverly, Mass.). The vectors used were the pET-30abc series of expression vectors (Novagen, Madison, Wis.) that permit the cloning of inserts in each of the three reading frames under the transcriptional control of the T7 phage promoter. The restriction enzyme-digested plasmid DNA was gel-purified using the QIAEX II Gel Extraction Kit (Qiagen, Valencia, Calif.), and then treated with shrimp alkaline phosphatase. Genomic DNA of an O157 isolate from one of the four HUS patients was partially digested with the restriction enzyme, Sau3AI. Following fractionation on a 1% agarose gel, DNA fragments ranging in size from ca. 0.5-1.5 kbp (insert DNA) were excised and purified using the QIAEX Gel Extraction Kit (Qiagen). Various ratios of insert and vector DNA were ligated and used to transform competent E. coli DH5α via electroporation according to standard protocols (Sambrookand Russel 2001. A Laboratory Manual. Cold Spring Harbor Laboratory Press, Cold Spring Harbor, N.Y.). Transformants were plated on LB plates supplemented with 50 μg/ml of kanamycin (LB-kan). After an overnight incubation at 37° C., growth was scraped off the plates, plasmid DNA isolated using standard procedures (Ibid), and used to transform electro-competent E. coli BL21(DE3) (Novagen), a general purpose expression host. To determine the percentage of transformants containing inserts, the library was plated on LB-kan and 100 colonies were randomly picked and analyzed by colony PCR using vector-specific primers. Greater than 90% of transformants contained inserts ranging in size from 0.2 kbp-1.8 kbp.

Screening of the Expression Library and Identification of Clones Expressing Immunogenic O157 Proteins.

The expression library was first screened with pooled, unadsorbed HUS-convalescent sera as follows. An optimal dilution of the library in E. coli BL21 (DE3) was plated on LB-kan to yield ca. 300-350 colonies per plate. After 5 h of incubation at 37° C., colonies were lifted using a nitrocellulose filter, and placed colony side up on fresh LB-Kan plates containing 1 mM isopropyl-β-D-thiogalactoside. Plates were incubated overnight at 30° C. to induce expression of genes contained within cloned inserts. Colonies on plates were partially lysed by exposing them to chloroform vapors for 15 min in a candle jar. The filters were removed from the plates, air dried and blocked using 5% non-fat milk in PBS (pH 7.4) for 1 h at room temperature. After rinsing with PBS-T, the filters were probed with a 1:500 dilution of pooled, unadsorbed HUS-convalescent sera for 1 h at room temperature on a rocking platform. Filters were washed 3× with PBS-T, and incubated with a 1:5000 dilution of peroxidase-labeled goat IgG directed against human gamma globulin fraction (ICN/Cappel). Following development with an ECL chemiluminescence kit (Amersham Pharmacia Biotech), reactive clones were identified by their positions on the reference plate (the original plate from which the colonies were lifted that was also incubated overnight at 30° C.). Each reactive clone identified in the primary screen was purified further, and tooth-picked in a grid pattern such that each test clone alternated with E. coli BL21 (DE3) (pET-30a), the negative control, and processed as described above.

Identification of O157 Proteins Expressed Exclusively During Human Infection Using IVIAT.

To identify proteins expressed by O157 exclusively during human infection and not during in vitro growth, the clones identified above were subjected to IVIAT using pooled, adsorbed HUS-convalescent sera. Clones were tooth-picked onto LB-kan plates in a grid pattern, and incubated for 5 h at 37° C. Processing of filters and screening were identical to that described above, except that a 1:100 dilution of pooled, adsorbed HUS-convalescent sera was used as a probe. Following confirmation by 4 additional rounds of screening, plasmid inserts were sequenced and encoded proteins were identified via BLAST against the genomic sequence of E. coli O157:H7 EDL 933 and Sakai strains. Proteins expressed from inserts within positive clones were called in vivo-induced proteins (ivi-proteins), and the genes encoding them were referred to as ivi-genes. Positive clones were further probed with individual, adsorbed, HUS-convalescent serum samples from each of the four patients, and the control serum sample, as described above.

Cellular localization of ivi-proteins was predicted using PSORT/PSORT-B program. Hypothetical ivi-proteins were assigned putative functions using the Clusters of Orthologous Groups (COGs) database available at. The online browser tool, Proteome Navigator, was used to compare proteins not assigned specific functions by the COGs database against Prolinks, a database of protein functional linkages derived from co-evolution (Bowers et al. 2004. Genome Biol 5:R35.

Proteomic Analysis of O157 Grown in LB Broth Using Microcapillary High Performance Liquid Chromatography Combined with Electrospray Ionization Tandem Mass Spectrometry (ESIμLC-MS/MS).

To confirm that the ivi-proteins were not expressed when O157 was cultured under standard laboratory conditions, O157 grown in LB broth were subjected to ESIμLC-MS/MS, at the Harvard Partners Center for Genetics and Genomics, Cambridge, Mass. The O157 whole cells for one dimensional microcapillary high performance liquid chromatography-tandem mass spectrometry (1D μLC-MS/MS) analysis were prepared as follows: mid-log phase O157 (OD₆₀₀=0.7) cultured at 37° C. in LB broth were pelleted via centrifugation at 4° C. and washed twice in deionized water. Cells were aliquotted into 1.5 mL tarred centrifuge tubes, frozen at −80° C. and lyophilized to dryness under high vacuum. The tubes were then weighed again and the total dried cell pellet weight determined. Dried O157 pellets (2 mg) were dissolved in 200 μL of 6M urea, 1% SDS 100 mM ammonium bicarbonate 10 mM DTT (pH 8.5). Samples were vortexed, and following incubation at 37° C. for 1 hour, 12 μL of 500 mM iodoacetamide, 100 mM ammonium bicarbonate pH 8.5 was added to each 200 μL sample. The reaction was allowed to proceed at room temperature for 1 hour in the dark. Alkylation was quenched by the addition of 2 μL of 2M DTT in 100 mM ammonium bicarbonate pH 8.5. Samples were then diluted 8 fold with 5 mM CaCl2, mixed with 20 μg of Promega sequencing grade trypsin, and incubated at 37° C. for 16 hours. Following quenching with 2 μL of formic acid, samples were diluted with 2 mL of 0.1% formic acid and cleaned up using a Waters Oasis MCX cartridge. Peptides were eluted with 6% ammonium hydroxide in 50% acetonitrile, frozen and lyophilized. Samples were redissolved in 5% acetonitrile 0.1% formic acid/water and loaded onto a 96 well plate for MS analysis.

For mass spectrometry, samples were run on a LCQ DECA XP plus Proteome X workstation from Thermo Finnigan. For each run, 85 μL of reconstituted sample was injected with a Surveyor Autosampler, while the separation was done on a 250 μm i.d.×30 cm column packed with C18 media running at a 2 μL per minute flow rate provided from a Surveyor MS pump with a flow splitter, with a gradient of 5-72% water 0.1% formic acid, acetonitrile 0.1% formic acid over the course of 240 min (4 hour). Two such runs were performed. Between each set of samples, two standards of a 5 Angio mix of peptides (Michrom BioResources) were run to ascertain column performance, and observe any potential carryover that might have occurred. The LCQ was run in a top five configuration, with one MS scan and five tandem MS (MS/MS) scans. Dynamic exclusion was set to 1 with a limit of 30 seconds. Peptide ID's were made using SEQUEST™ (Thermo Finnigan) through the Bioworks Browser 3.1. Sequential database searches were made using the E. coli O157:H7 strain EDL933 FASTA database from the EMBL European Bioinformatics institute using differential carbamidomethyl modified cysteines and oxidized methionines. A yeast protein database was spiked in to provide noise and determine validity of the peptide hits. In this fashion, known and theoretical protein hits can be found without compromising the statistical relevance of all the data (Peng et al. 2003. J Proteome Res 2:43-50). Peptide score cutoff values were chosen at Xcorr of 1.8 for singly charged ions, 2.5 for doubly charged ions, and 3.0 for triply charged ions, along with deltaCN values of 0.1, and RSP values of one. The cross correlation values chosen for each peptide assured a high confidence match for the different charge states, while the deltaCN cutoff insured the uniqueness of the peptide hit; RSP value of one insured that the peptide matched the top hit in the preliminary scoring.

### Results and Discussion

Pooled, Unadsorbed HUS-Convalescent Sera Reacted Specifically with Previously Identified, Immunogenic O157 Proteins.

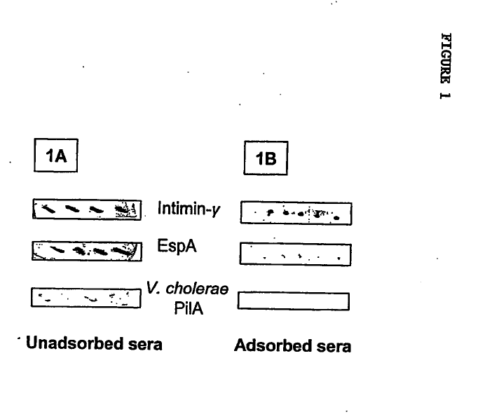

Pooled, unadsorbed HUS-convalescent sera reacted strongly and specifically with E. coli XL1-Blue (pEB313) (McKee et al. 1996. Infect Immun 64:2225-2233) and E. coli DH5α (pCVD468, pREP4) (Karpman et al. 2002. Pediatr Nephrol 17:201-211), expressing His₆-intimin-γ and His₆-EspA, respectively, in contrast to E. coli BL21(DE3) expressing recombinant Vibrio cholerae PilA, an irrelevant control protein (FIG. 1A). This suggested that the pool of sera possessed sufficient reactivity for probing the expression library. On the other hand, the unabsorbed healthy control serum did not react with either of these proteins (data not shown).

Adsorption of Pooled, HUS-Convalescent Sera as Per the IVIAT Protocol Resulted in Selective Depletion of Antibodies Against O157 Antigens Expressed in Vitro.

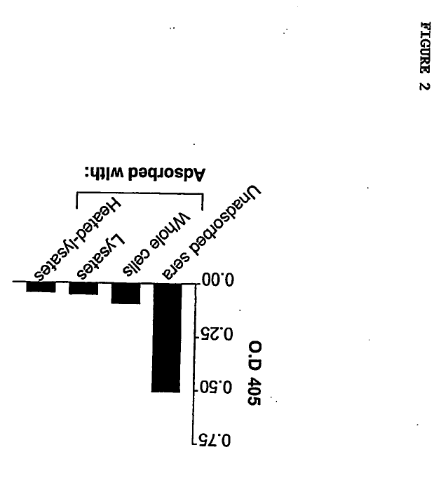

Adsorption efficiency was determined by examining the reactivity of serum aliquots from pooled, HUS-convalescent sera after each adsorption step with in vitro grown O157 lysates. There was a sharp decline in reactivity of sera against the in vitro grown O157 lysate following the first adsorption step, compared to the unadsorbed sera, indicating efficient depletion of antibodies against in vitro-expressed O157 proteins (FIG. 2).

Although the adsorbed sera continued to react with the clone expressing His₆-intimin-γ, it did not react with the clone expressing His₆-EspA (FIG. 1B). Since both are reportedly expressed during human infection (Ibid; Li et al. 2000. Infect Immun 68:5090-5095), as well as during in vitro growth (McNally et al. 2001. Infect Immun 69:5107-5114), we anticipated reactivity with both might be eliminated by adsorption of the sera. We attribute residual serum reactivity with intimin-γ to relatively weak in vitro expression of this protein, which may be insufficient to adsorb away all of the anti-intimin-γ antibodies generated in response to significantly higher expression of this adhesin within the gastrointestinal tract.

Screening of an O157 Genomic Expression Library.

Primary screening of ca.50,000 clones of an O157 genomic expression library, using pooled, unadsorbed HUS-convalescent sera, yielded 918 reactive clones. IVIAT of these clones using pooled, adsorbed HUS-convalescent sera identified 223 persistently reactive clones containing unique inserts as determined from non-redundant databases.

IVI-Proteins Included Previously Identified O157 Virulence-Related Proteins.

IVIAT identified proteins previously reported to have a putative role in O157 virulence ((Besser et al. 1999. Annu Rev Med 50:355-367); Table 1). These included: (i) intimin-γ, the LEE-encoded outer membrane adhesin that acts in concert with other LEE-encoded proteins to generate the attaching-effacing lesion (Kaper et al. 2004. Nat Rev Microbiol 2:123-140), and binding to host nucleolin (Sinclair and O'Brien 2002. J Biol Chem 277:2876-2885) to tether the bacterium to the enterocyte; (ii) QseA, a backbone-encoded LysR-type quorum sensing E. coli transcriptional regulator, which is part of the regulatory cascade that controls expression of O157 virulence factors via quorum sensing (Sperandio et al. 2002. Mol Microbiol 43:809-821). QseA is also present in other gastrointestinal pathogens such as enteropathogenic E. coli (EPEC) (QseA), and V. cholerae (AphB). Following activation by the furanone AI-2, QseA activates transcription of Ler, the positive activator of the LEE operon, and thereby influences expression of putative virulence factors from the LEE. A qseA mutant is impaired in the secretion of LEE-encoded proteins via the Type III secretion system also encoded on the LEE (Sperandio et al. 2002. Infect Immun 70:3085-3093); (iii) TagA, a pO157-encoded inner membrane lipoprotein (Paton et al. 2002. J Clin Microbiol 40:1395-1399). TagA is a protein of unknown function but a putative role in O157 virulence has been suggested because of its presence in a diverse collection of O157 strains (Paton et al. 2002. J Clin Microbiol 40:1395-1399) and the fact that a homolog in V. cholerae is regulated by ToxR, a transcriptional regulator that governs expression of several V. cholerae virulence factors (Harkey et al. 1995. Gene 153:81-84); and (iv) MsbB2, a pO157-encoded inner membrane acyl transferase, which facilitates the synthesis of hexaacyl lipid A, the form with maximal biological activity (Kim et al. 2004. Infect Immun 72:1174-1180). MsbB2 reportedly functions to suppress minor modifications of Lipid A. Acting in conjunction with MsbB1, another homologous acyltransferase expressed from the chromosome, MsbB2 facilitates the synthesis of lipid A of maximal biological activity that interacts optimally with the subject immune system to evoke an immune response to LPS (Ibid). Strongly supporting a role for MsbB2 in O157 virulence is the fact that LPS reportedly acts synergistically with the Shiga toxins, especially Stx2, in the pathogenesis of HUS (Ikeda and Honda 2004. Pediatr Nephrol 19:485-489). Further support for a likely role in O157 virulence is suggested by observations that MsbB2 contributes to virulence of related pathogens such as Shigella flexneri and septicemic E. coli O18:K1:H7 strain H16, and that it influences expression of virulence-related surface structures in diverse pathogens (Kim et al. 2004. Infect Immun 72:1174-1180).

Ivi-Proteins Expressed from the O157 Backbone.

A total of 181 ivi-proteins of diverse functional classes were expressed from the backbone (Table 2). Those involved in biosynthesis and metabolism (51.38%) may have functions essential for bacterial growth in vivo, a feature imperative for bacterial pathogenicity. Also, consistent with the anaerobic gut environment, IVIAT identified glycolytic enzymes, hydrogenases involved in fermentation of carbon compounds, an alcohol dehydrogenase, and reductases (including two cryptic nitrate reductases) involved in energy generation from carbohydrates via anaerobic respiration and fermentation (Peekhaus and Conway 1998. J Bacteriol 180:3495-3502). These results were expected and consistent with that of other studies of diverse organisms using techniques such as transcriptional profiling (Xu et al. 2003. Proc Nat Acad Sci USA 100:1286-1291), in vivo expression technology (WET) (Heithoff et al. 1997. Trends Microbiol 5:509-513; Mahan et al. 2000. Amu Rev Genet 34:139-164), recombinase in vivo expression technology (RIVET) (Camilli and Mekalanos 1995. Mol Microbiol 18:671-683), signature-tagged mutagenesis (STM) (Hava et al. 2002. Mol Microbiol 45:1389-1405; Mei et al. 1997. Mol Microbiol 26:399-407; Merrell et al. 2002. Mol Microbiol 43:1471-1491), selective capture of transcribed sequences (SCOTS) (Dozois et al. 2003. Proc Nat Acad Sci USA 100:247-252) and IVIAT of other organisms (Cao et al. 2004. FEMS Microbiol Lett. 237:97-103).

Transport ivi-proteins (18.78%), included diverse ABC-type transporters and phosphotransferase systems (PTS), permeases, transport proteins involved in the transport of diverse molecules, iron-uptake proteins such as FhuA (a transporter of ferrichrome (Fe3+) and a receptor for phages and colicins), CirA, an iron-regulated receptor for uptake and transport of colicin I, uncharacterized transport proteins, and in particular, an anaerobically-induced permease, TdcC, expressed from the tdc operon that encodes transport and degradation of L-threonine and L-serine. Inactivation of the activator, TdcA, results in hyperadherence of O157 to cultured epithelial cells due to derepression of OmpA expression (Torres and Kaper 2003. Infect Immun 71:4985-4995).

Regulatory proteins (13.81%) comprised among others, QseA, a LysR-type transcriptional activator (described above) (Sperandio et al. 2002. Mol Microbiol 43:809-821), and several two-component regulatory systems that govern virulence of diverse pathogens (Hoch and Silhavy 1995. Two-Component Signal Transduction, ASM Press, Washington, D.C.). Some of these are functionally interlinked as evidenced by earlier reports (Groisman. 2001. J. Bacteriol. 183:1835-1842; Hagiwara et al. 2003. J. Bacteriol. 185:5735-5746; Heithoff et al. 1999. J. Bacteriol. 181:799-807; Wosten et al. 2000. Cell 103:113-125) and the Prolinks database, suggesting IVIAT identified proteins that sense and integrate diverse environmental signals (such as anaerobiosis, cation limitation, acid, and excess, toxic levels of extracytoplasmic Fe³⁺), and help O157 mount a coordinated cellular adaptive response to counter the hostile host environment. The two-component regulatory systems IVIAT identified were, (i) the sensor molecule, NarX, of the NarL-NarX system, which in the absence of oxygen responds to nitrate or nitrite and acts via NarL, the response regulator that activates expression of enzymes involved in nitrate respiration and represses enzymes involved in respiration of other electron acceptors (Lee et al. 1999. J Bacteriol 181:5309-5316; Rabin and Stewart 1992. Proc Nat Acad Sci USA 89:8419-8423); (ii) the sensor kinase component, PhoQ, of the PhoP-PhoQ system, which responds to extracytoplasmic levels of Mg2+ and Ca2+ (involved in the adaptation to Mg2+ limitation) (Groisman. 2001. J. Bacteriol. 183:1835-1842), and to Zn2+ excess (Hagiwara et al. 2003. J. Bacteriol. 185:5735-5746); (iii) the sensor component, BasS, of the BasR-BasS system that governs the response to excess extracytoplasmic Fe3+ (Wosten et al. 2000. Cell 103:113-125) and mild acid pH (Soncini and Groisman 1996. J Bacteriol 178:6796-6801); (iv) the sensor protein, GlnL, of the GlnG-GlnL system that responds to low ammonia concentration and stimulates ammonia assimilation (Neidhardt et al. 1996. Escherichia coli and Salmonella: Molecular and Cellular Biology. American Society for Microbiology, Washington, D.C.); and (v) HydH, the sensor for HydG, which primarily responds to high periplasmic Zn2+ and Pb2+, and nonspecifically activates the expression of hydrogenase 3, an enzyme involved in hydrogen production during fermentation (Leonhartsberger et al. 2001. J Mol Biol 307:93-105). IVIAT also identified an outer membrane lipoprotein, RcsF, that transduces a signal in response to glucose and zinc to the RcsC/YojN/RcsB/RcsA phosphorelay system, which in turn controls the rcs regulon (target genes), encoding enzymes for colanic acid exopolysaccharide capsule (Hagiwara et al. 2003. J. Bacteriol. 185:5735-5746), an acid-adaptive response that protects O157 from environmental stresses to acid and heat (Mao et al. 2001. J Bacteriol 183:3811-3815). Other ivi-proteins that were part of this functional group and likely to impact in vivo survival of O157 were: (i) PrpR, a regulator of the prp operon involved in catabolism of propionate, a short chain fatty acid (SCFA) that can be detrimental to O157 in high concentrations (exposure to SCFA is a stress condition and catabolism may serve to decrease the concentration of this short chain fatty acid (Kwon and Ricke 1998. Appl Environ Microbiol 64:3458-3463; Polen et al. Appl Environ Microbiol 69:1759-1774); (ii) FucR, a positive regulator of the fuc operon encoding enzymes for metabolism of L-fucose, a component of both mucus and glycans on enterocytes (Peekhaus and Conway 1998. J Bacteriol 180:3495-3502). Following experimental inoculation of mice, O157 reportedly is found attached to both mucus and enterocytes (in contrast to non pathogenic E. coli, which is found in mucus alone) (Miranda et al. 2004. Infect Immun 72:1666-1676), and may utilize L-fucose as one nutrient source to multiply and out-compete other flora to establish infection; (iii) FliS and FliT, which along with FliD negatively regulate the export of the anti-sigma factor, FlgM, to prevent expression of the flagellar regulon (which may promote in vivo survival, since over production of flagella is deleterious to bacterial growth) (Yokoseki et al. 1996. J Bacteriol 178:899-901), and (iv) paraquat-inducible protein A (PqiA), an inner membrane protein of uncharacterized function.

Ivi-proteins functioning in environmental adaptation (8.84%) included methyl accepting chemotaxis proteins (MCPs), a protein that was part of the adaptive response to hyperosmolarity, a colicin expressed in response to iron-limiting conditions, a modulator of drug activity, and two proteins of the PhoB regulon expressed as part of the adaptive response to phosphate limitation. Specifically, IVIAT identified two methyl accepting chemotaxis proteins (MCPs), namely Trg, a receptor for the periplasmic ribose and galactose binding proteins, and Tsr, the serine sensor receptor, both of which are regulators of chemotaxis and motility (Neidhardt et al. 1996. Escherichia coli and Salmonella: Molecular and Cellular Biology. American Society for Microbiology, Washington, D.C.). Interestingly, MCPs were also highly expressed during human infection with V. cholerae as identified by IVIAT (Hang et al. 2003. Proc Nat Acad Sci USA 100:8508-8513). IVIAT also identified OsmY, a periplasmic protein of unknown function that is induced in response to hyperosmolarity (Yim et al. 1992. J Bacteriol 174:3637-3644). This protein is expressed as part of the Rcs regulon, which includes genes encoding the synthesis of the exo-polysaccharide colanic acid capsule (see above), and possibly functions in the transport of an alternative osmolyte (Hagiwara et al. 2003. J. Bacteriol. 185:5735-5746). One of the ivi-proteins in this subgroup was CirA, an iron-regulated receptor for colicin homologous to a siderophore iron-uptake system, which is also expressed in iron-limiting conditions by other pathogens such as Salmonella typhimurium (Heithoff et al. 1999. J. Bacteriol. 181:799-807). Interestingly, as in previous studies (Ibid; Wosten et al. 2000. Cell 103:113-125), IVIAT identified proteins expressed in response to iron limitation (FhuA and CirA), as well as those expressed in response to extracytoplasmic iron excess (BasS of the BasR-BasS two-component system; see above). Other ivi-proteins included MdaA, a modulator of drug activity, and two proteins that are part of the Pho regulon and expressed as part of the adaptive response to limiting phosphate in the environment, namely, PhoE, an outer membrane porin functioning in the transport of various anions, and a periplasmic, phosphate ester hydrolase, PhoA, involved in the degradation of nontransportable organophosphates (Neidhardt et al. 1996. Escherichia coli and Salmonella: Molecular and Cellular Biology. American Society for Microbiology, Washington, D.C.). The PhoB regulon is required for colonization of the rabbit small intestine by V. cholerae (von Kruger et al. 1999. Microbiol 145:2463-2475), and regulates hilA and invasion genes in S. typhimurium (Lucas et al. 2000. J Bacteriol 182:1872-1882). A possible role in O157 virulence is also suggested by the fact that in vivo expression of PhoB in an avian pathogenic E. coli strain during experimental infection of chickens was identified by SCOTS (Dozois et al. 2003. Proc Nat Acad Sci USA 100:247-252).

Phage-related proteins (1.11%) included an inner membrane protein of unknown function, and a non-specific protease that degrades the lambda repressor, cII. Those of unknown function (6.08%) rounded off ivi-proteins expressed from the backbone. Collectively, these results suggested that defined backbone ivi-proteins not only support pathogenicity by facilitating in vivo survival, but also regulate and indirectly complement pathogen-specific virulence factors.

Ivi-Proteins Expressed from O-Islands (OIs).

The 37 ivi-proteins expressed from OIs included 13 phage-related proteins (Table 3). Because phage proteins include both Stx1 and Stx2 in EHEC, and because they also include proteins that influence every stage of infection of mammalian subjects by diverse pathogens (Wagner and Waldor 2002 Infect Immun. 70:3985-3993), they are potential virulence factors and warrant further evaluation. Although IVIAT did not identify either Stx1 or Stx2, (both are also produced during in vitro growth in LB broth) (Ritchie et al. 2003. Appl Environ Microbiol 69:1059-1066), it identified two homologous ivi-proteins of unknown function, one expressed from each of the phages that encode Stx1 and Stx2. These two ivi-proteins are homologous to ivi-proteins expressed from several other cryptic prophages (Table 3).

Particularly interesting was that certain OIs (#36 and #71) containing cryptic prophages expressing ivi-proteins, also expressed non-phage, non-LEE (Nle) effectors (proteins encoded outside the LEE, but secreted via the type III secretion apparatus encoded on the LEE) (Deng et al. 2004. Proc Nat Acad Sci USA 101:3597-3602). The presence of Nle homologs, such as NleA and NleF (OI #71) and NleB, NleC, and NleD (OI #36), in related pathogens, and the requirement of NleA for full virulence of C. rodentium, a pathogen of mice, suggest a probable role for phage proteins expressed from these OIs in O157 virulence (Gruenheid et al. 2004. Mol Microbiol 51:1233-1249).

IVIAT identified 24 clones whose inserts encoded proteins expressed from OI sequences not part of phage genomes (Table 3). These included intimin-γ expressed from the LEE (OI #148), WbdP, a cytoplasmic glycosyl transferase (OI #84) involved in the synthesis of the O-polysaccharide antigen (Wang and Reeves 1998. Infect Immun 66:3545-3551), WaaD, a putative periplasmic glycosyltransferase (OI #145) involved in biosynthesis of the oligosaccharide core of LPS, and numerous ivi-proteins with putative or unknown functions (Table 3). The identification of enzymes involved in both O-antigen and LPS core biosynthesis by IVIAT was expected because LPS is a broadly recognized virulence determinant of pathogenic Gram negative bacteria, and transcripts of genes encoding such enzymes were identified during infection of chickens with avian pathogenic E. coli (APEC) using SCOTS (7). Other non-phage ivi-proteins that could impact O157 virulence included a putative arylsulphatase (OI #40) involved in the scavenging of sulfate and implicated in the ability of E. coli K1 to invade brain microvascular endothelial cells (Hoffman et al. 2000. Infect Immun 68:5062-5067), and a putative recombinant hot spot A (RhsA) protein (OI #30) that reportedly contributes to genomic plasticity (Lin et al. 1984. J Mol Biol 177:1-18). Transcripts of a gene encoding RhsH, a similar protein, were also detected during APEC infection of chickens by SCOTS (Dozois et al. 2003. Proc Nat Acad Sci USA 100:247-252).

Many non-phage ivi-proteins were expressed from seven of the nine large OIs (>15 kb) that reportedly encode putative virulence factors (Table 4; 5). Besides intimin-γ from OI #148, IVIAT also identified a putative acyl-coenzyme A synthetase (fatty acid:CoA ligase) expressed from OI #138, which catalyzes the formation of fatty acyl-CoA, a substrate for phospholipid biosynthesis and enzymes of β-oxidation, and is involved in diverse functions such as protein transport, protein acylation, enzyme activation, cell signaling, and control of transcription (Neidhardt et al. 1996. Escherichia coli and Salmonella: Molecular and Cellular Biology. American Society for Microbiology, Washington, D.C.); a putative inner membrane ABC-type transport permease expressed from OI #47 and functioning in cell wall biogenesis; a putative, inner membrane ABC-type bacteriocin/lantibiotic exporter expressed from OI #28 that functions in the export of large molecules such as proteins and peptides, and homologous to ATP-binding proteins of ABC transporters and toxin secretion systems of several pathogens, including Pseudomonas putida, Salmonella typhi, and V. cholerae; a cytoplasmic esterase of the α-β hydrolase superfamily expressed from OI #43; a conserved cytoplasmic protein of unknown function expressed from OI #7; and an inner membrane protein of unknown function expressed from OI #48. Interestingly, IVIAT did not identify clones expressing putative virulence factors encoded on OI #115 and OI #122 (Table 4). Perhaps these proteins are expressed equally in vitro and in vivo, resulting in the removal of corresponding reactive antibodies during adsorption, or these proteins are not immunogenic, or antibodies to these proteins are short-lived.

Ivi-Proteins Expressed from pO157.

Ivi-proteins expressed from pO157 (Table 3), included the previously discussed TagA and MsbB2; SopA, an ATPase that accurately partitions low copy number F plasmids into daughter cells (also identified during APEC infection of chickens) (Dozois et al. 2003. Proc Nat Acad Sci USA 100:247-252); a putative nickase associated with plasmid maintenance; and a putative hemolysin expression modulating protein, a homolog (90% amino acid identity) of the E. coli regulator Hha (Burland et al. 1998. Nucl. Acids. Res. 26:4196-4204), which complexes with the nucleoid-associated, universal regulator protein, H—NS, and governs expression of the hly operon in response to changes in temperature and osmolarity (Madrid et al. 2002. J Bacteriol 184:5058-5066). In addition, Hha has also been shown to repress the LEE-encoded regulator (Ler) in O157, thereby causing reduced expression of the esp operon encoding the LEE translocator proteins, EspA, EspB, and EspD (Sharma and Zuerner 2004. J Bacteriol 186: 7290-7301).

Majority of Clones Expressing Ivi-Proteins Reacted Specifically and Broadly with HUS-Convalescent Serum from Individual Patients.

Ivi-proteins for practical applications such as the development of diagnostic markers, vaccines and drugs, should ideally be expressed strongly during infection and evoke robust immune responses broadly in patients with O157 disease. The majority of the 223 positive clones identified earlier using pooled, adsorbed HUS-convalescent sera, reacted with each of the four individual serum samples that made up the pool, but not with a control serum sample taken from a healthy pediatric patient (Tables 1, 2, 3). However, 15 ivi-proteins expressed from the backbone and four ivi-proteins expressed from OIs reacted differentially with individual patient serum. We are currently investigating via PCR whether the failure of individual patients to respond to a particular ivi-protein is attributable to heterogeneity of cognate isolates. Also, 22 backbone (Table 2) and two O157-specific ivi-proteins (Table 3) reacted with the control serum. We speculate that this may be due to cross-reacting antibodies in the control sera, or less likely, the presence of pre-existing antibodies to O157 proteins from prior, unrecognized infection. Studies are ongoing to compare reactivity of sera from healthy individuals, of different age groups, to the ivi-antigens.

Ivi-Proteins Expressed from OIs and pO157 were not Among the 300 O157 Proteins Most Highly Expressed During In Vitro Growth.

The central premise of IVIAT is that identified proteins are expressed during infection but not during growth under standard laboratory conditions. Proteomic analysis using ESIμLC-MS/MS confirmed that none of the 37 ivi-proteins expressed from OIs or five expressed from pO157 were among the 300 O157 proteins most highly expressed during growth in LB broth (data not shown). This was not entirely expected because many of the O157-specific proteins are reportedly expressed, at least to some degree, in vitro (McNally et al. 2001. Infect Immun 69:5107-5114). We speculate that owing to minimal expression of such proteins during in vitro culture, a mass spectrometry run of longer duration would be required for their identification.

In contrast, 18 of 181 backbone ivi-proteins were expressed sufficiently in LB broth for detection by proteomic analysis (Table 5). Of these 18 backbone ivi-proteins expressed in vitro, 12 were identified by ESIμLC-MS/MS during the course of both runs; six were weakly expressed in one run only, at a percent protein abundance ranging from 0.09 to 0.04 (Table 5). We hypothesize that these 18 proteins are expressed at higher levels during human infection than during growth in LB broth and attribute their identification by IVIAT to the fact that low-level protein expression during in vitro growth may not effectively deplete HUS-convalescent sera of antibodies against these ivi-proteins during absorption.

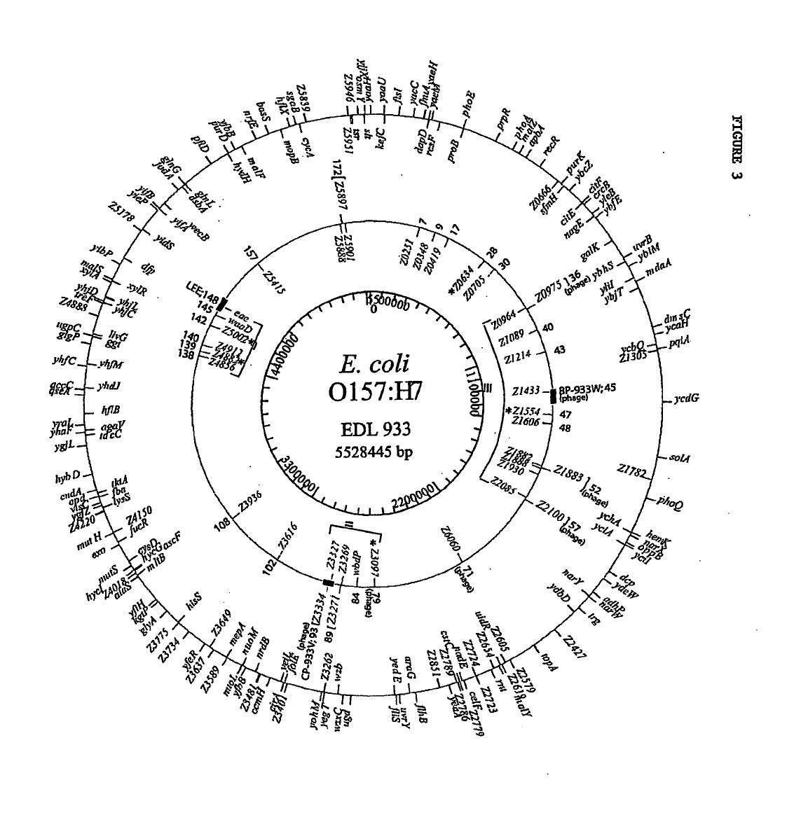

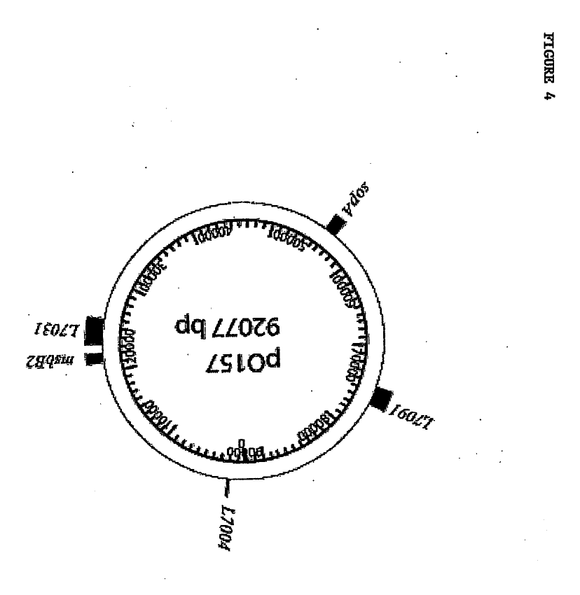

A graphical representation of the locations of ivi-genes on the O157 chromosome is shown in FIG. 3 and on the pO157 plasmid in FIG. 4. Ivi-genes included 181 of 4029 (4.5%) open reading frames (ORFs) in the backbone, 37 of 1387 (2.7%) ORFs in the OIs, and 5 of 100 (5%) ORFs in pO157 sequences of the EDL933 genome (Perna et al. 2001. Nature 409:529-533).

The 181 backbone-specific ivi-genes were distributed uniformly on the O157 chromosome; however, several of the 37 OI-specific ivi-genes appeared to localize in three discrete OI groups, (a group contained four or more OIs, with each separated by five or fewer intervening OIs). Although in most cases only one ivi-gene mapped to an individual OI, there were instances where multiple ivi-genes (two or more) mapped within the same OI (OI #36, OI #52, OI #57, OI #89, OI #93). The five ivi-genes that mapped to pO157 are also shown in FIG. 4.

The apparent grouping of OIs expressing ivi-proteins raises the possibility that ivi-proteins (and other proteins) expressed from OIs within a particular group might act in concert to optimally influence a specific function. Particularly interesting was that group I, which included OI #148 expressing the adhesin, intimin-γ, also included OI #145 expressing a glycosyltransferase, WaaD, one of the many enzymes involved in the biosynthesis of LPS core oligosaccharide. The fact that WaaD is expressed from the same operon as WaaI (another enzyme that functions in LPS core oligosaccharide biosynthesis), and that O157 waaI deletion mutants are hyper-adherent to cultured intestinal epithelial cells (Torres and Kaper 2003. Infect Immun 71:4985-4995) may suggest that during human infection, intimin-γ, LPS of O157 (and possibly other ivi and non ivi-proteins, expressed from OIs within this group) might act in concert to modulate adherence of O157 to human epithelial cells. It will be interesting to test this hypothesis experimentally and also to determine whether proteins in OI groups II and III might be functionally related as well.

In conclusion, IVIAT identified 223 O157 proteins expressed in vivo during human infection, several of which were unique to this study. Although IVIAT for O157 was validated by the identification of previously identified potential O157 virulence factors, prior infection with O157 does not necessarily produce full protection from subsequent re-infection (Besser et al. 1999. Annu Rev Med 50:355-367). This may reflect suboptimal antibody responses to protective antigens, and we hypothesize that robust expression and optimal delivery of relevant ivi-proteins (and other O157 antigens) to the mucosal immune system might engender more protective immune responses. Preliminary experiments demonstrated that all of the 223 reactive clones also reacted with pooled, adsorbed sera from patients who had recovered from hemorrhagic colitis (data not shown) suggesting that similar pathogenic mechanisms may be operating in this illness and in HUS.

IVIAT provides a “snap shot” of O157 protein expression during infection, and a glimpse of the possible mechanisms by which this pathogen might counter host defenses, and adapt and establish itself within the human gut to cause disease. Studies directed toward the characterization of the role of ivi-proteins in O157 pathogenesis are currently underway. The identification of ivi-genes unique to diverse O157 isolates, as well as non-O157 EHEC, and Shiga toxigenic E. coli (STEC) that lack LEE but are pathogenic to humans, and of ivi-genes shared between EHEC and EPEC, augurs well for the future development of diagnostic tests for EHEC and STEC infection, as well as the development of common drugs and vaccines against EHEC, STEC and EPEC.

### Example 2

### E. coli Antigens Identified in Cattle

The primary route for human infection with E. coli is through contaminated beef products. Therefore an effective means for reducing E. coli disease in humans is the vaccination of cattle against E. coli. Using PELS, described herein, we have identified immunogenic E. coli proteins that are expressed in cattle.

Proteins constituting microbial immunomes (the subset of microbial antigens that elicit subject immune responses) have excellent diagnostic, prophylactic and therapeutic potential, since a subset of such immunogenic proteins is part of the repertoire of microbial factors that function to help pathogens counter subject defenses, facilitate niche-adaptation and survive and replicate in these subjects. Methodologies to rapidly identify such protein targets facilitate exploitation of microbial genome sequence data, and expedite the development of novel management strategies against infectious diseases.

Traditional methodologies for proteome-wide identification of immunogenic microbial proteins (IMPs) involve screening microbial recombinant genomic expression libraries in plasmid/phage expression vectors and laboratory host strains, with sera from colonized or infected subjects. However, colony immunoscreening and in vivo-induced antigen technology (IVIAT) (Rollins et al. 2005. Cell Microbiol 7:1-9), a variation of colony immunoscreening that defines only partial immunoproteomes, and bacterial surface display coupled with magnetic cell sorting (Etz et al. 2002. Proc Nat Acad Sci USA 99:6573-6578) are laborious, and require several months or more for definitive IMP-identification. Immunoproteomics of pathogens cultured in vitro under either standard laboratory conditions or those that attempt to mimic the host environment are also popular; however, pathogens cultured in vitro might not express the entire spectrum of virulence proteins. In view of the challenging task of accurately reproducing the host environment, such approaches might overlook those immunogenic virulence proteins that are expressed exclusively in response to host environmental cues, and contribute significantly to pathogenicity (Mahan et al. 2000. Annu Rev Genet. 34:139-164). Although these limitations may be circumvented by immunoproteomics of pathogens isolated directly from either biological specimens or subject anatomical sites of infection, consistent recovery of sufficient numbers of suitable organisms for analysis presents a significant challenge (Xu et al. 2003. Proc Nat Acad Sci USA 100: 1286-1291). Protein microarray/chip-technology has tremendous potential for rapid, global definition of IMPS, but is constrained by bottlenecks in proteome-scale purification of microbial proteins, and currently permits immunological characterization of only a partial proteome (Li et al. 2005. Infect Immun 73:3734-3739). Newer formats such as nucleic acid programmable protein arrays (NAPPA) (Ramachandran et al. 2004. Science 305:86-90) are still experimental, and neither NAPPA nor antibody arrays/protein chips utilizing surface enhanced laser desorption/ionization time of flight (SELDI-TOF) mass spectrometry for antigen identification (Hess et al. 2005. J Chromatogr B Analyt Technol Biomed Life Sci 815:65-75) have been demonstrated to rapidly define microbial immunoproteomes.

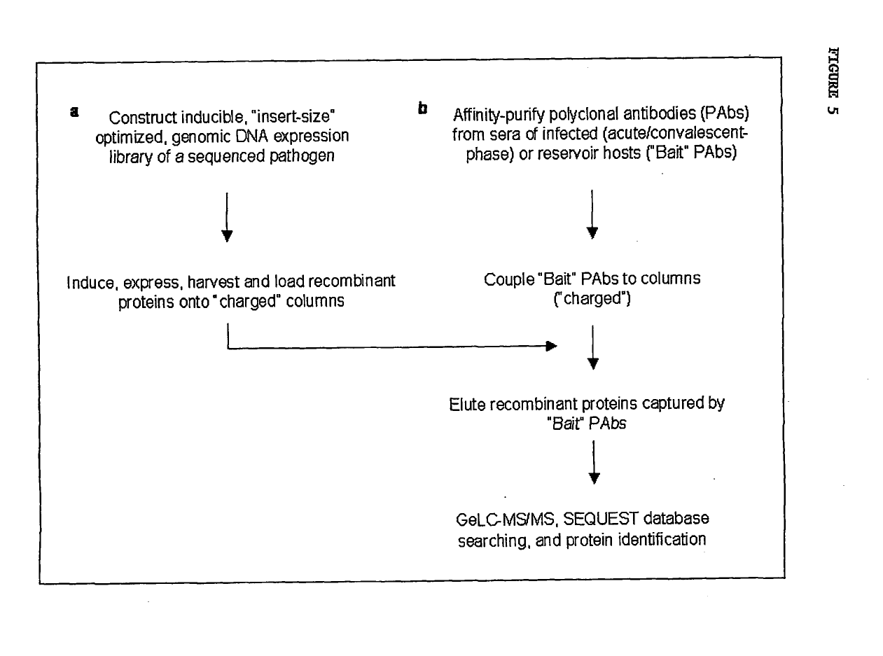

To rapidly identify proteins comprising microbial immunomes, we have developed a novel technique, PELS, which couples standard recombinant DNA and immunochemistry techniques with proteomics. The principle of PELS is outlined in FIG. 5, and involves capture of recombinant proteins expressed from an inducible, microbial genomic DNA expression library using polyclonal antibodies (PAbs) affinity-purified from acute/convalescent sera of infected subjects or sera from reservoirs colonized by the cognate pathogen (bait PAbs), coupled to a solid support. Proteins captured by the bait PAbs are subjected to 1D SDS PAGE ESI nano LC-MS/MS (GeLC-MS/MS), and identified via SEAQUEST database searching (Steen and Mann 2004. Nat Rev Mol Cell Biol 5:699-711) (FIG. 5). The entire process, from recombinant genomic expression library construction to definitive protein identification, is accomplished in only three weeks without biases inherent to manual screening. To our knowledge, this is the first application of proteomics for rapid, global identification of IMPs from among proteins expressed from genes on inserts within recombinant clones comprising microbial genomic expression DNA libraries.

### Materials and Methods

Recombinant DNA Methods and Proteomic Analysis.

Isolation of plasmid DNA, restriction digestions, and agarose gel electrophoresis were performed using standard procedures (Sambrookand Russel 2001. A Laboratory Manual. (Cold Spring Harbor Laboratory Press, New York). Enzymes for restriction digestions, DNA modifications and ligations were procured from New England Biolabs, Beverly, Mass. Oligonucleotides for PCR were obtained from the DNA Synthesis Core Facility, Department of Molecular Biology, Massachusetts General Hospital. Plasmids were electroporated into E. coli DH5α or BL21(DE3) using a Gene Pulser (Bio-Rad Laboratories, Richmond, Calif.) as instructed by the manufacturer. Electroporation conditions were 2,500 V at 25-mF capacitance, producing time constants of 4.8 to 4.9 ms. Proteomic analysis, namely, 1D SDS-PAGE nano capillary high-performance liquid chromatography combined with electrospray ionization tandem mass spectrometry (1D SDS-PAGE ESI nano LC-MS/MS or GeLC-MS/MS), was performed at the Harvard Partners Center for Genetics and Genomics, Cambridge, Mass.

Construction of an Inducible, Escherichia coli O157:H7 (O157) Genomic DNA Expression Library Comprising of Clones Containing DNA Inserts of Optimal Size (“Optimized” O157 Expression Library).

We generated an O157 genomic DNA expression library as described above, using genomic DNA isolated from the O157 strain 43894 (an isolate from a human patient with hemorrhagic colitis in the United States) in the pET-30abc series of expression vectors (Novagen, Madison, Wis.). Vector and insert DNA in the size range of 0.5 to 3.0 kbp were prepared as described previously (Ibid). Because accuracy of protein identification using tandem MS/MS data and SEQUEST database searching increases with the number of peptides generated following trypsin digestion of the cognate protein (Steen and Mann 2004. Nat Rev Mol Cell Biol 5:699-711), we sought to preferentially ligate insert DNA fragments that were larger than the average size of ORFs in this pathogen. The rationale was that larger DNA fragments were more likely to include genes encoding full-length or near full-length recombinant proteins containing potentially a larger number of recognition sites for cleavage by trypsin, such that multiple peptide fragments resulting from trypsin cleavage of such proteins might facilitate robust protein identification. We accomplished this by performing multiple ligation reactions, with each containing a different mole ratio of insert to vector. Each ligation reaction was then used to transform competent E. coli DH5α via electroporation according to standard protocols (Sambrookand Russel 2001. A Laboratory Manual. (Cold Spring Harbor Laboratory Press, New York). To minimize overrepresentation of sister clones in the O157 expression library, transformants were directly plated onto LB plates supplemented with 50 μg/ml of Kan (LB-Kan) without allowing for phenotypic expression, and incubated overnight at 37° C. To determine both the percentage of transformants containing inserts as well as the insert size, 100 colonies were randomly picked from each O157 expression library, and analyzed by colony PCR using vector-specific primers. Greater than 90% of all transformants examined contained inserts, and >80% of clones of one O157 expression library included inserts that exceeded 1.7 kbp in size. Recombinant clones comprising this particular O157 expression library were scraped off LB-Kan plates, plasmid DNA was isolated using the QIAprep Spin Miniprep Kit (Qiagen Sciences, MD), and used to transform electrocompetent E. coli BL21(DE3) (Novagen), the recommended expression host, and plated as described above. The resultant O157 expression library comprised >10⁵ recombinant clones. Given the genome sizes of the sequenced O157 strains EDL933 (Perna et al. 2001. Nature 409:529-533), and Sakai (Hayashi et al. 2001. DNA Res 8:11-22), and that the average size of open reading frames (ORFs) in these strains is 1 kbp (Ohnishi et al. 1999. DNA Res 6:361-368), we concluded that this expression library, comprising of recombinant clones containing inserts of optimal size (referred to herein as the “optimized” O157 expression library), was adequate for defining components of the O157 immunoproteome (Stephenson 2005. A Guide to Mathematics in the Laboratory. (Academic Press, Massachusetts).

Expression of the Optimized O157 Expression Library and Preparation of Cell-Lysate and Pellet Fractions Containing Recombinant Proteins for Immunoaffinity Capture.

To expedite sample preparation, and circumvent laborious experimentation to define the inducer concentration and post induction growth conditions (such as incubation temperature and duration of growth after induction) that facilitated recombinant O157 protein expression in a form that permitted optimal capture by polyclonal antibodies (PAbs) affinity purified from pooled hyperimmune cattle sera (referred to herein as “bait” PAbs), and coupled to HiTrap NHS-activated HPcolumns (see below), several batches of the “optimized” O157 expression library were cultured simultaneously at 37° C. to an initial OD₆₀₀ of 0.6. Cultures were then induced with 0.25 mM, 0.5 mM, 1 mM or 2.0 mm isopropyl-β-D-thiogalactopyranoside (IPTG), and incubated at 25° C., 30° C., or 37° C. for either 5 h or overnight. Cultures were pooled, centrifuged at 7,000 rpm for 10 min at 4° C. to harvest cell pellets, which were then washed 3× with chilled, sterile PBS (pH 7.4). Complete® protease inhibitor cocktail (Roche Diagnostics, Indianapolis, Ind.) was added to twice the concentration recommended by the manufacturer, after which cells were lysed by three cycles of freeze-thaw. Lysed cells were resuspended in 2 ml chilled PBS, microcentrifuged at maximum speed for 10 min, and supernatant (lysate) fractions decanted into fresh tubes. Pellet fractions were resuspended in 2 ml of PBS containing 2×“Complete” protease inhibitor cocktail (Roche), and 0.2% (final concentration) of the nonionic detergent, n-octyl-β-D-glucopyranoside (NOG), was added to solubilize membrane proteins. Both the lysate and pellet fractions containing recombinant O157 proteins were stored at −70° C. until used.

Coupling of Bait PAbs to HiTrap NHS-Activated HP Columns.

Prior to coupling, hyperimmune cattle sera against diverse O157 strains was generated, and evaluated for reactivity against previously identified O157 antigens (Supplemental material). Polyclonal antibodies were affinity-purified from pooled, hyperimmune cattle sera (Bait PAbs) as detailed herein. Affinity-purified Bait PAb fractions were pooled together and dialyzed overnight against PBS (pH 7.4) at 4° C., and then for 4 h against coupling buffer consisting of 0.2 M NaHCO3, 0.5 M NaCl (pH 8.3) using a dialysis membrane with a molecular weight cut off of 3500. Coupling of bait PAbs via amine groups was done as instructed by the manufacturer with minor modifications. Following removal of isopropanol, HiTrap NHS-activated columns were equilibrated with ten column volumes of coupling buffer. Pooled, bait IgG PAbs were slowly loaded on the column using a syringe and then recirculated through the column for 30 min at room temperature by attaching another syringe to the outlet. Active groups that did not couple to the ligand were quenched with 10 ml of 1 M Tris (hydroxymethyl)aminomethane (pH 9.0), and nonspecifically bound PAbs were eluted with 10 ml of 1 M acetic acid. Columns with immobilized bait PAbs (charged columns) were then rinsed with 10 ml of deionized water to remove the quencher and the eluant.

Capture of Recombinant O157 Proteins.

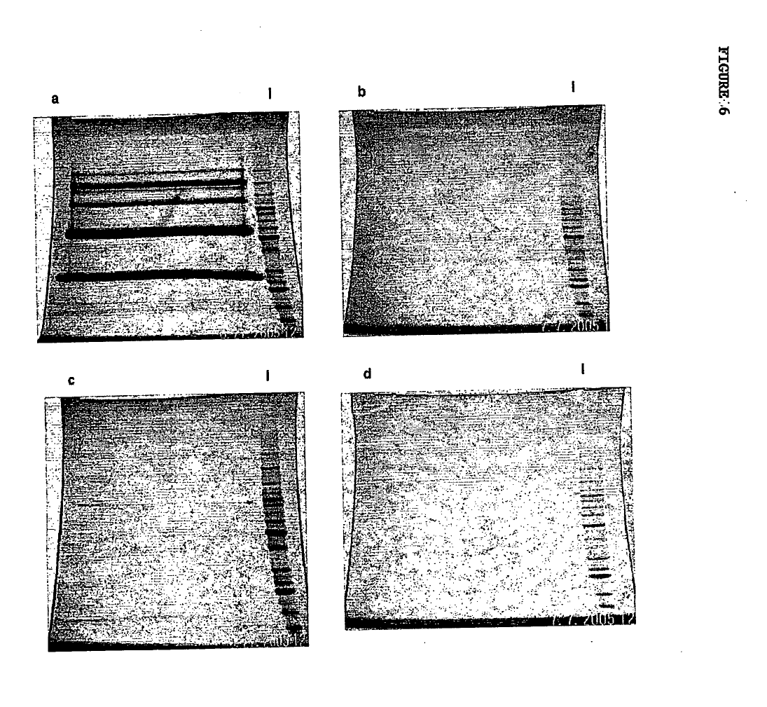

“Charged” columns were equilibrated with ten column volumes of binding buffer consisting of PBS (pH 7.4), 0.2% NOG. Cell lysates and pellet fractions containing recombinant O157 proteins from the previous step were diluted with PBS-0.2% NOG containing 2× concentration of “Complete” protease inhibitor cocktail (Roche Diagnostics) in 20 ml, and then loaded via a syringe separately onto the charged columns, at a flow rate dictated by gravity (˜0.75-1 ml/min). Following a rinse with 20 volumes of loading buffer to remove loosely bound proteins, specifically captured recombinant proteins were eluted with 10 ml of 1 M acetic acid directly into 15 ml Falcon tubes containing 500 μl of ammonium hydroxide. This process was repeated three times to maximize yield of captured recombinant O157 proteins. Nonspecific adsorption of recombinant O157 proteins to the column matrix was assessed by passing “optimized” O157 expression library lysate and pellet fractions of cells of the O157 expression library through “uncharged” columns (HiTrap NHS-activated HP columns not coupled to bait PAbs), with quenched active groups. Specificity of capture and lack of nonspecific adsorption was confirmed by visualizing recombinant O157 proteins by fractionation of elutions from “charged” and “uncharged” columns on SDS-PAGE gels and subsequent staining (FIG. 6A-D).

Proteomic Analysis of Elutions from “Charged” and “Uncharged” Columns Using 1D SDS Page ESI Nano LC-MS/MS (GeLC-MS/MS).

Elutions from charged and uncharged columns were subjected to 1D SDS PAGE followed by nanocapillary high-performance liquid chromatography combined with electrospray ionization tandem mass spectrometry (ESI nano LC-MS/MS) (GeLC-MS/MS; see below) at the Harvard Partners Center for Genetics and Genomics, Cambridge, Mass. The advantages of GeLC-MS/MS over LC-MS/MS have been well documented (Steen and Mann 2004. Nat Rev Mol Cell Biol 5:699-711). In preparation for 1D SDS-PAGE, elutions were concentrated using spin filters (MW cutoff 5000 Daltons) (Vivascience Inc., Edgewood, N.Y.), and reduced by incubation in 250 μl of 8M urea/100 mM ammonium bicarbonate/1% SDS/10 mM DTT at 37° C. for 1 h. Following a further incubation at room temperature for 20 min, each sample was alkylated by the addition of 15 μl of 500 mM iodoacetamide, and incubation at room temperature in the dark for 60 min. Alkylation was then quenched with 3 μl of 2M DTT to each sample. Following the addition of 150 μl of SDS-PAGE loading buffer per tube, each sample was centrifuged at 14,000 rpm for 10 min at room temperature, and 400 μl of each sample was fractionated on a 1D SDS-PAGE Tris-Glycine 8-16% gradient (Invitrogen) for 2.5 h at 125 volts, 20 mA and 8 Watts. Gels were alternatively shrunk for 12 h by the addition of 500 ml of 50% methanol, 5% acetic acid, and then allowed to swell up for 1 h by the addition of 500 ml of deionized water for 60 min on a rotary shaker. Gels were then stained with SimplyBlue Safe Stain (Invitrogen) for 14-16 h on a rotary shaker, imaged and sliced horizontally (using molecular weight standards as a guide) into multiple sections of equal size, and processed as described below.

In-Gel Digestion/Peptide Extraction.

Gel sections from the previous step were placed in 2.0 ml tubes (Axygen, Union city, Calif.), and moved into a dual isolation biosafety cabinet. Gel pieces were destained with 2 washes of 50% methanol, 5% acetic acid, and rinsed with three alternating washes of 100 mM ammonium bicarbonate and 100% acetonitrile to remove the destain solution. Following removal of the acetonitrile after the final wash, gel slices were dried for 10 minutes in a speedvac. Tubes containing the dried gel sections were placed on ice, and 200 μL of Promega sequencing grade trypsin at a concentration of 6.6 μg/ml in 50 mM ammonium bicarbonate was added to each sample. The gel pieces were allowed to swell for 60 minutes on ice, after which the tubes were capped and incubated at 37° C. for 20 hours. Peptides were extracted with two washes of 500 μL of 50 mM ammonium bicarbonate and two washes of 500 μL of 50% acetonitrile, 0.1% formic acid. All extracts were frozen at −80° C., lyophilized to dryness, and redissolved in 60 μl of 5% acetonitrile, 0.1% formic acid. Samples were then loaded onto a 96 well plate for mass spectrometry (MS) analysis (see below).

Mass Spectrometry (MS).

Samples for MS were run on either an LCQ DECA XP (LCQ) plus Proteome X workstation as described above, or on a linear ion trap-Fourier transform mass spectrometer (LTQ-FT) from Thermo Finnigan. For each run using the LCQ, 10 μL of each reconstituted sample was injected with a Famos Autosampler, while the separation was done on a 75 μm (inner diameter) by 20 cm column packed with C18 media running at a flow rate of 250 nanoliters (nL) per minute provided from a Surveyor MS pump with a flow splitter with a gradient of 5-72% water 0.1% formic acid, and 5% acetonitrile over the course of 240 min (4.0 hour run). For each run using the LTQ-FT, 10 μL of each reconstituted sample was injected with a Famos Autosampler, while the separation was done on a 75 μm (inner diameter)×20 cm column packed with C18 media running at a 225 nL/minute flow rate provided from a Surveyor MS pump with a flow splitter with a gradient of 5-60% water 0.1% formic acid, acetonitrile 0.1% formic acid over the course of 120 minutes (150 rain total run). Between each set of samples, 2.5 h standards of a 5 Angio mix of peptides (Michrom BioResources) were run to ascertain column performance, and observe any potential carryover that might have occurred. The LCQ was run in a top five configuration with one MS scan and five MS/MS scans. Dynamic exclusion was set to 1 with a limit of 30 seconds. The LTQ-FT was run in a top nine configuration, with one MS 200K resolution full scan and nine MS/MS scans. Dynamic exclusion was set to 1 with a limit of 180 seconds with early expiration set to 2 full scans.

Peptide identifications were made using SEQUEST (Thermo Finnigan) through the Bioworks Browser 3.2. Sequential database searches were performed using the E. coli O157:H7 strain EDL933 FASTA database from European Bioinformatics Institute using static carbamidomethyl-modified cysteines and differential oxidized methionines. A reverse E. coli O157:H7 strain EDL933 FASTA database was spiked in to provide noise and determine validity of peptide hits. In this fashion, the statistical relevance of all the data could be determined in a sample and mass spectrometer independent manner (Peng et al. 2003. J Proteome Res 2:43-50). LCQ data was searched with a 2 Dalton window on the MS precursor with 0.8 Dalton on the fragment ions, while the FT data was searched at 5 ppm for the precursor ion and 0.5Da on the fragment ions. Peptide score cutoff values were chosen at cross-correlation values (Xcorr) of 1.8 for singly charged ions, 2.5 for doubly charged ions, and 3.0 for triply charged ions, along with delta rank scoring preliminary cutoff (deltaCN) values of 0.1, peptide probability values of 1.00 E-3 and cross-correlation normalized values (RSP) of <10. The cross-correlation values chosen for each peptide assured a high confidence match for the different charge states, while the deltaCN ensured the uniqueness of the peptide hit. The RSP value of 1 was used on single peptide hits to ensure that the peptide matched the top hit in the preliminary scoring. At these peptide filter values, very few single peptide reverse database hits were observed, allowing us to place a higher confidence on the few single peptide protein identifications. Single hit proteins, were also manually validated to ensure relevance.

Cellular localization and putative functions of hypothetical proteins identified by querying the E. coli O157:H7 strain EDL933 FASTA database at European Bioinformatics Institute with tandem MS data were determined using bioinformatics as follows: (i) cellular localization of such proteins was determined using the PSORTb v.2.0/PSLpred and PSORTdb databases; (ii) extracytoplasmic location was confirmed by examining the N-terminus of amino acid sequences of cognate proteins from the O157 strain EDL 933 database from The Institute for Genome Research, for the presence of a signal sequence using the program SignalP 3.0, and (iii) putative functions were designated using the NCBI Conserved Domain Database (CDD).

### Results and Discussion

Generation of Hyper-Immune Bovine Sera.

Hyperimmune sera were obtained from cattle experimentally inoculated with E. coli O157 strains of both cattle and human origin (Torres and Kaper 2003. Infect Immun 71:4985-4995), which included the strain EDL933, one of the sequenced O157 strains (Hayashi et al. 2001. DNA Res 8:11-22; Torres and Kaper 2003. Infect Immun 71:4985-4995). The rationale was that exposure of these cattle to diverse O157 strains of both lineage I and II (Tarr et al. 2000. Infect Immun 68:1400-1) might engender antibody responses against a wider variety of protein antigens expressed in the gastrointestinal tract (GIT) of cattle rendering the hyperimmune sera optimal for profiling the immunoproteome of this organism in the bovine reservoir. Cattle were first confirmed to be negative for O157 colonization using highly sensitive recto-anal swab culture techniques (Torres and Kaper 2003. Infect Immun 71:4985-4995), and then inoculated once orally with 10¹⁰ CFU of each strain (Ibid). Serum samples, collected from nine cattle that had remained culture positive for up to two months (Ibid) following experimental inoculation, demonstrated high titers of anti-O157 lipopolysaccharide (LPS) antibodies (Ibid). Preimmune sera were also collected from the same animals prior to inoculation.

Preliminary Evaluation of Reactivity of Pooled Hyperimmune Sera.

We assessed the quality of pooled hyperimmune sera, by reacting them with previously identified, secreted O157 proteins encoded in the locus of enterocyte effacement (LEE) (Sambrookand Russel 2001. A Laboratory Manual. (Cold Spring Harbor Laboratory Press, New York). Cattle immunized with such proteins, including those expressed from the LEE, reportedly show decreased shedding of O157 (Ibid). We first confirmed reactivity of hyper-immune cattle sera with O157 lipopolysaccharide (LPS) purified in our laboratory using a protocol described herein (FIG. 6 a and b). We then amplified genes encoding full-length EspB from O157 strain EDL933 (test), and PilA from Vibrio cholerae E1 Tor N16961 (control), and cloned them into the expression vector, pET-30b (Novagen, Inc., Madison, Wis.) under control of the phage T7 promoter. Following confirmation of in frame cloning via gene sequencing, we transformed plasmids into E. coli BL21(DE3). Recombinant clones were induced with isopropyl-β-D-thiogalactopyranoside (IPTG) and probed in a colony immunoblot assay with pooled, hyper-immune cattle sera adsorbed against purified O157 LPS, as described previously by our group (Perna et al. 2001. Nature 409:529-533). None of the above recombinant clones reacted with pooled, pre-immune cattle sera.

Affinity-Purification of Pabs from Hyperimmune Sera.

Prior to affinity purification, we pooled hyperimmune sera from nine cattle to compensate for variations in individual immune responses and identify a wider complement of O157 proteins expressed within the GIT of bovine reservoirs. PAbs from 1 ml of pooled hyperimmune cattle sera were purified using HiTrap Protein G HP (5 ml) columns (Amersham Biosciences, Piscataway, N.J.), as recommended by the manufacturer with a few modifications. Briefly, pooled sera were diluted 1:4 in binding buffer (0.02 M Sodium phosphate buffer, pH 7.0), and then loaded using a syringe onto a protein G column equilibrated with ten volumes of binding buffer. Protein G binds all IgG subclasses but not other immunoglobulin isotypes (Johnson et al. 2005. Infect Immun 73:965-971). Following a wash with ten volumes of binding buffer, bound IgG PAbs were eluted with 4 ml of elution buffer (0.1 M Glycine, pH 2.7), directly into five tubes each containing 200 μl of 1M Tris-HCl, pH 9.0. Affinity-purified IgG PAbs (bait PAbs) were quantified using a nomograph (Dziva et al. 2004. Microbiol. 150:3631-3645) and prepared for coupling.

The PELS-Identified O157 Immunoproteome in Bovine Reservoirs.

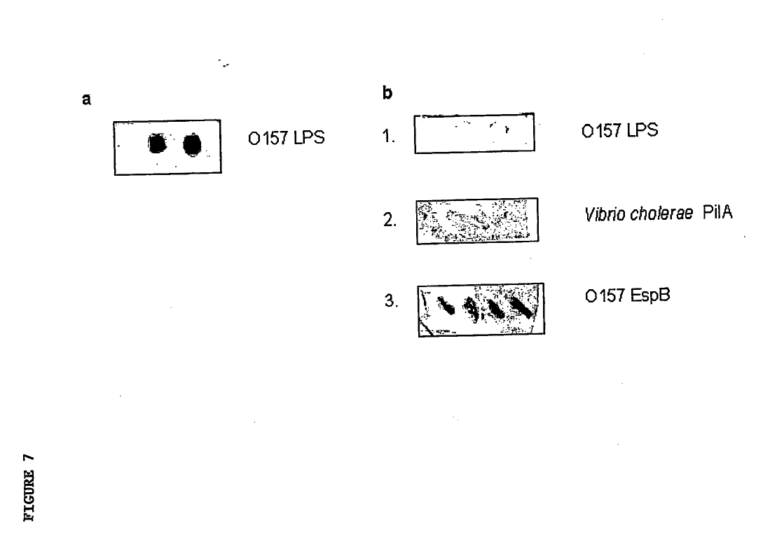

In order to validate PELS (FIG. 5), we sought to rapidly define a protein-subset constituting the O157 immunome in bovine reservoirs as a vaccine for elimination of this pathogen from the gastrointestinal tracts (GIT) of cattle, a principal source of human infection. For this, we constructed and induced recombinant protein expression from genes on inserts within clones comprising an “optimized” O157 expression library using a range of IPTG concentrations, followed by growth at different incubation temperatures for varying time intervals. To capture recombinant proteins contained in cell-lysate and pellet fractions of library clones, we affinity-purified bait PAbs from pooled, hyperimmune cattle sera previously generated against diverse O157 strains following confirmation of reactivity of this hyperimmune cattle serum pool against previously identified O157 antigens (FIGS. 7A and B). We then “charged” HiTrap NHS-activated columns by coupling to bait PAbs, after which pooled cell-lysate and pellet fractions from above were applied separately to “charged” columns. Specifically captured O157 recombinant proteins were identified by subjecting pooled elution-fractions to GeLC-MS/MS and SEQUEST database searching (Tables 6 and 7).

PELS identified 207 proteins, comprising 3.8% of the proteome of the sequenced O157 strain EDL933 (Perna et al. 2001. Nature 409:529-533), as components of the immunome of this organism in bovine reservoirs (Tables 6 and 7). PELS was strongly validated by the fact that 35 of 207 (17%) proteins were also part of the O157 immunome in humans convalescing from extraintestinal O157 disease. Further validation of PELS came from identification of previously identified adhesins of O157 (Torres and Kaper 2003. Infect Immun 71:4985-4995; Tarr et al. 2000. Infect Immun 68:1400-1) and of other pathogens (Johnson et al. 2005. Infect Immun 73:965-971) (Table 6), as well as several bovine colonization factors (Dziva et al. 2004. Microbiol. 150:3631-3645) (Table 7), which was consistent with the fact that this human pathogen only colonizes the GIT of cattle, but does not cause disease in these reservoirs. As in the earlier report on the O157 immunome in humans identified by IVIAT, PELS identified outer membrane components of high-affinity iron transport, bacteriophage proteins, biosynthetic and metabolic enzymes, and proteins involved in energy generation and anaerobic respiration, in accordance with requirements for adaptation to the host environment, in vivo growth and survival. PELS also identified several hypothetical and unknown proteins, including those unique to this study (Table 7).

### Example 3

### Identification of Antigens from Other Pathogens

The PELS method described herein (Example 2), is useful for identifying immunogenic pathogen-derived proteins from virtually any pathogenic organism. Examples of pathogenic organisms are bacteria, fungi, viruses, annelids, nematodes, platyhelminthes, and protozoans. Examples of pathogenic bacteria include, without limitation, Aerobacter, Aeromonas, Acinetobacter, Agrobacterium, Bacillus, Bacteroides, Bartonella, Bortella, Brucella, Calymmatobacterium, Campylobacter, Citrobacter, Clostridium, Cornyebacterium, Enterobacter, Enterococcus, Escherichia, Francisella, Haemophilus, Hafnia, Helicobacter, Klebsiella, Legionella, Listeria, Morganella, Moraxella, Proteus, Providencia, Pseudomonas, Salmonella, Serratia, Shigella, Staphylococcus, Streptococcus, Treponema, Xanthomonas, Vibrio, and Yersinia.

In the example of M. tuberculosis, the method of the invention is used to identify immunogenic proteins. A recombinant DNA expression library representing the entire genome of M. tuberculosis is used to produce recombinant proteins. These proteins are contacted with polyclonal antibodies that are purified from the sera of human subjects infected with M. tuberculosis. Proteins that form complexes with these antibodies are isolated, and the identity of the immunogenic proteins is determined through GeLC-MS/MS and SEQUEST database searching.

Examples of pathogenic fungi include, without limitation, Alternaria, Aspergillus, Basidiobolus, Bipolaris, Blastoschizomyces, Candida, Candida albicans, Candida krusei, Candida glabrata (formerly called Torulopsis glabrata), Candida parapsilosis, Candida tropicalis, Candida pseudotropicalis, Candida guilliermondii, Candida dubliniensis, and Candida lusitaniae, Coccidioides, Cladophialophora, Cryptococcus, Cunninghamella, Curvularia, Exophiala, Fonsecaea, Histoplasma, Madurella, Malassezia, Plastomyces, Rhodotorula, Scedosporium, Scopulariopsis, Sporobolomyces, Tinea, and Trichosporon.

### Example 4

### Cattle Vaccination

Proteins identified in any of the above screens (Examples 1-3) are evaluated as vaccines for prevention of E. coli O157:H7 colonization of the bovine gastrointestinal tract, including the recto-anal junction (RAJ) (Naylor et al. 2003. Infect Immun 71:1505-1512) according to standard methods known in the field. The immunogenic composition of such an experimental vaccine comprise a single or a combination of proteins that are administered as either a protein or a DNA vaccine.

In preparation for a protein-based vaccine, any of the proteins described herein (Tables 2, 3, 7, and SEQ ID NOs: 1-340) are purified using standard techniques. Various formulations of immunogens comprising the vaccine are evaluated in cattle with and without adjuvants (Harlow and Lane 1988. Cold Spring Harbor Laboratory Press, Cold Spring Harbor, N.Y.). In order to determine the optimal method of delivery, the protein vaccine is administered via diverse routes of immunization, including subcutaneous, intradermal, and intramuscular. Various formulations of the protein vaccine may also be administered using transcutaneous immunization (TCI) (Glenn et al. 1999. Infect. Immun 67:1100-1106) in conjunction with ADP-ribosylating toxins and other adjuvants (Scharton-Kersten et al. 2000. Infect Immun 68:5306-5313) for induction of robust mucosal and systemic immune responses.

In preparation for a plasmid DNA (pDNA) based vaccine, the gene encoding the entire open reading frame of the cognate protein will amplified from E. coli O157:H7 genomic DNA via PCR, its nucleic acid sequence confirmed, and subsequently cloned into suitable pDNA vaccine vectors. Following confirmation of protein expression by SDS-PAGE-coomassie blue staining of extracts of cultured mammalian cells, various formulations of the pDNA-based vaccine will be evaluated with and without adjuvants in cattle. As for protein-based experimental vaccines, the pDNA-based genetic vaccine will be administered via topical skin application, subcutaneous, intradermal, intramuscular routes. These vaccines will also be administered using a biolistic device such as a gene gun (Fynan et al. 1993. Proc Nat Acad Sci USA 90:11478; Johnston and Tang 1994. Methods Cell Biol 43 Pt A:353-65).

Cattle are primed and boosted (immunized) with the protein or pDNA-based experimental vaccine alone or may be primed with one experimental vaccine and boosted with the other. Cattle primed on Day 0 will be boosted three times at 14-day intervals. Pre-immune sera will be collected on Day 0, and on days 14, 28, 42, for measurement of immune responses. Cattle will be challenged by oral inoculation of >10⁹ each of a diverse collection E. coli O157:H7 at starting from 14 days after the third booster. The efficacy of the experimental vaccine will be determined by decreased duration of shedding in stools (relative to that in unimmunized controls), and an absence of colonization of the RAJ of experimentally inoculated, immunized cattle (Naylor et al. 2003. Infect Immun 71:1505-1512).

### Example 5

### Additional Embodiments

Based on the nucleotide and amino acid sequences described herein, the isolation of additional coding sequences of virulence factors is made possible using standard strategies and techniques that are well known in the art. Any pathogenic cell can serve as the nucleic acid source for the molecular cloning of such a virulence gene, and these sequences are identified as ones encoding a protein exhibiting pathogenicity-associated structures, properties, or activities.

In one particular example of such an isolation technique, any one of the nucleotide sequences described herein may be used, together with conventional screening methods of nucleic acid hybridization screening. Such hybridization techniques and screening procedures are well known to those skilled in the art. In one particular example, all or part of any one of the nucleotide sequences identified in Tables 2, 3, or 7 and SEQ ID NOs: 1-340, may be used as a probe to screen a recombinant DNA library for genes having sequence identity to such genes. Hybridizing sequences are detected by plaque or colony hybridization according to standard methods.

Alternatively, using all or a portion of the amino acid sequence of any one of the polypeptides identified in Tables 2, 3, or 7 and SEQ ID NOs: 1-340, one may readily design specific oligonucleotide probes, including degenerate oligonucleotide probes (i.e., a mixture of all possible coding sequences for a given amino acid sequence). These oligonucleotides may be based upon the sequence of either DNA strand and any appropriate portion of the sequence of the polypeptide. General methods for designing and preparing such probes are known in the art. These oligonucleotides are useful for gene isolation, either through their use as probes capable of hybridizing to complementary sequences or as primers for various amplification techniques, for example, polymerase chain reaction (PCR) cloning strategies. If desired, a combination of different, detectably-labelled oligonucleotide probes may be used for the screening of a recombinant DNA library. Such libraries are prepared according to methods well known in the art.

As discussed above, sequence-specific oligonucleotides may also be used as primers in amplification cloning strategies, for example, using PCR. PCR methods are well known in the art and are described. Primers are optionally designed to allow cloning of the amplified product into a suitable vector, for example, by including appropriate restriction sites at the 5′ and 3′ ends of the amplified fragment (as described herein). If desired, nucleotide sequences may be isolated using the PCR “RACE” technique, or Rapid Amplification of cDNA Ends. By this method, oligonucleotide primers based on a desired sequence are oriented in the 3′ and 5′ directions and are used to generate overlapping PCR fragments. These overlapping 3′- and 5′-end RACE products are combined to produce an intact full-length cDNA.

Partial virulence sequences, e.g., sequence tags, are also useful as hybridization probes for identifying full-length sequences, as well as for screening databases for identifying previously unidentified related virulence genes.

Confirmation of a sequence's relatedness to a pathogenicity polypeptide may be accomplished by a variety of conventional methods including, but not limited to, functional complementation assays and sequence comparison of the gene and its expressed product. In addition, the activity of the gene product may be evaluated according to any of the techniques described herein, for example, the functional or immunological properties of its encoded product.

Once an appropriate sequence is identified, it is cloned according to standard methods and may be used, for example, for screening compounds that reduce the virulence of a pathogen.

### Polypeptide Expression

In general, polypeptides of the invention may be produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.). The method of transformation or transfection and the choice of expression vehicle will depend on the host system selected. Transformation and transfection methods are standard methods; expression vehicles may be chosen from those known in the art.

One particular bacterial expression system for polypeptide production is the E. coli pET expression system (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains which express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system which is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3× may be cleaved with factor Xa.

Once the recombinant polypeptide of the invention is expressed, it is isolated, e.g., using affinity chromatography. In one example, an antibody (e.g., produced as described herein) raised against a polypetide of the invention may be attached to a column and used to isolate the recombinant polypeptide. Lysis and fractionation of polypeptide-harboring cells prior to affinity chromatography may be performed by standard methods.

Once isolated, the recombinant protein can, if desired, be further purified, e.g., by high performance liquid chromatography (see, e.g., Fisher, Laboratory Techniques In Biochemistry And Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.).

These general techniques of polypeptide expression and purification can also be used to produce and isolate useful peptide fragments or analogs (described herein).

### Antibodies

To generate antibodies, a coding sequence for a polypeptide of the invention may be expressed as a C-terminal fusion with glutathione S-transferase (GST) (Smith et al., 1988 Gene 67:31-40). The fusion protein is purified on glutathione-Sepharose beads, eluted with glutathione, cleaved with thrombin (at the engineered cleavage site), and purified to the degree necessary for immunization of rabbits. Primary immunizations are carried out with Freund's complete adjuvant and subsequent immunizations with Freund's incomplete adjuvant. Antibody titres are monitored by Western blot and immunoprecipitation analyses using the thrombin-cleaved protein fragment of the GST fusion protein. Immune sera are affinity purified using CNBr-Sepharose-coupled protein. Antiserum specificity is determined using a panel of unrelated GST proteins.

As an alternate or adjunct immunogen to GST fusion proteins, peptides corresponding to relatively unique immunogenic regions of a polypeptide of the invention may be generated and coupled to keyhole limpet hemocyanin (KLH) through an introduced C-terminal lysine. Antiserum to each of these peptides is similarly affinity purified on peptides conjugated to BSA, and specificity tested in ELISA and Western blots using peptide conjugates, and by Western blot and immunoprecipitation using the polypeptide expressed as a GST fusion protein.

Alternatively, monoclonal antibodies which specifically bind any one of the polypeptides of the invention are prepared according to standard hybridoma technology (see, e.g., Kohler et al., 1975 Nature 256:495; Kohler et al., 1976 Eur. J. Immunol. 6:511; Kohler et al., 1976., Eur. J. Immunol. 6:292; Hammerling et al., 1981 In Monoclonal Antibodies and T Cell Hybridomas, Elsevier, N.Y.,; Ausubel et al., supra). Once produced, monoclonal antibodies are also tested for specific recognition by Western blot or immunoprecipitation analysis (by the methods described in Ausubel et al., supra). Antibodies which specifically recognize the polypeptide of the invention are considered to be useful in the invention; such antibodies may be used, e.g., in an immunoassay. Alternatively monoclonal antibodies may be prepared using the polypeptide of the invention described above and a phage display library (Vaughan et al., 1996 Nature Biotech 14:309-314).

Preferably, antibodies of the invention are produced using fragments of the polypeptide of the invention which lie outside generally conserved regions and appear likely to be antigenic, by criteria such as high frequency of charged residues. In one specific example, such fragments are generated by standard techniques of PCR and cloned into the pGEX expression vector (Ausubel et al., supra). Fusion proteins are expressed in E. coli and purified using a glutathione agarose affinity matrix as described in Ausubel et al. (supra). To attempt to minimize the potential problems of low affinity or specificity of antisera, two or three such fusions are generated for each protein, and each fusion is injected into at least two rabbits. Antisera are raised by injections in a series, preferably including at least three booster injections.

Antibodies against any of the polypeptides described herein may be employed to treat bacterial infections.

### Screening Assays

As discussed above, we have identified a number of E. coli polypeptides that are expressed during human infection and that may therefore be used to screen for compounds that reduce the virulence of that organism, as well as other microbial pathogens. For example, the invention provides methods of screening compounds to identify those which enhance (agonist) or block (antagonist) the action of a polypeptide or the gene expression of a nucleic acid sequence of the invention. The method of screening may involve high-throughput techniques.

Any number of methods are available for carrying out such screening assays. According to one approach, candidate compounds are added at varying concentrations to the culture medium of pathogenic cells expressing one of the nucleic acid sequences of the invention. Gene expression is then measured, for example, by standard Northern blot analysis, using any appropriate fragment prepared from the nucleic acid molecule as a hybridization probe. The level of gene expression in the presence of the candidate compound is compared to the level measured in a control culture medium lacking the candidate molecule. A compound which promotes a decrease in the expression of the pathogenicity factor is considered useful in the invention; such a molecule may be used, for example, as a therapeutic to combat the pathogenicity of an infectious organism.

If desired, the effect of candidate compounds may, in the alternative, be measured at the level of polypeptide production using the same general approach and standard immunological techniques, such as Western blotting or immunoprecipitation with an antibody specific for a pathogenicity factor. For example, immunoassays may be used to detect or monitor the expression of at least one of the polypeptides of the invention in a pathogenic organism. Polyclonal or monoclonal antibodies (produced as described above) which are capable of binding to such a polypeptide may be used in any standard immunoassay format (e.g., ELISA, Western blot, or RIA assay) to measure the level of the pathogenicity polypeptide. A compound which promotes a decrease in the expression of the pathogenicity polypeptide is considered particularly useful. Again, such a molecule may be used, for example, as a therapeutic to combat the pathogenicity of an infectious organism.

Alternatively, or in addition, candidate compounds may be screened for those which specifically bind to and inhibit a pathogenicity polypeptide of the invention. The efficacy of such a candidate compound is dependent upon its ability to interact with the pathogenicity polypeptide. Such an interaction can be readily assayed using any number of standard binding techniques and functional assays. For example, a candidate compound may be tested in vitro for interaction and binding with a polypeptide of the invention and its ability to modulate pathogenicity may be assayed by any standard assays.

In one particular example, a candidate compound that binds to a pathogenicity polypeptide may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the pathogenicity polypeptide is identified on the basis of its ability to bind to the pathogenicity polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to render a pathogen less virulent (e.g., as described herein). Compounds isolated by this approach may also be used, for example, as therapeutics to treat or prevent the onset of a pathogenic infection, disease, or both. Compounds which are identified as binding to pathogenicity polypeptides with an affinity constant less than or equal to 10 mM are considered particularly useful in the invention.

Potential antagonists include organic molecules, peptides, peptide mimetics, polypeptides, and antibodies that bind to a nucleic acid sequence or polypeptide of the invention and thereby inhibit or extinguish its activity. Potential antagonists also include small molecules that bind to and occupy the binding site of the polypeptide thereby preventing binding to cellular binding molecules, such that normal biological activity is prevented. Other potential antagonists include antisense molecules.

Each of the DNA sequences provided herein may also be used in the discovery and development of antipathogenic compounds (e.g., antibiotics). The encoded protein, upon expression, can be used as a target for the screening of antibacterial drugs. Additionally, the DNA sequences encoding the amino terminal regions of the encoded protein or Shine-Delgarno or other translation facilitating sequences of the respective mRNA can be used to construct antisense sequences to control the expression of the coding sequence of interest.

The invention also provides the use of the polypeptide, polynucleotide, or inhibitor of the invention to interfere with the initial physical interaction between a pathogen and mammalian host responsible for infection. In particular the molecules of the invention may be used: in the prevention of adhesion and colonization of bacteria to mammalian extracellular matrix proteins; to extracellular matrix proteins in wounds; to block mammalian cell invasion; or to block the normal progression of pathogenesis.

The antagonists and agonists of the invention may be employed, for instance, to inhibit and treat a variety of bacterial infections.

Optionally, compounds identified in any of the above-described assays may be confirmed as useful in conferring protection against the development of a pathogenic infection in any standard animal model (e.g., the mouse-burn assay described herein) and, if successful, may be used as anti-pathogen therapeutics (e.g, antibiotics).

### Pharmaceutical Therapeutics

The invention provides a simple means for identifying compounds (including peptides, small molecule inhibitors, and mimetics) capable of inhibiting the pathogenicity or virulence of a pathogen. Accordingly, a chemical entity discovered to have medicinal or agricultural value using the methods described herein are useful as either drugs, vaccines, or as information for structural modification of existing anti-pathogenic compounds, e.g., by rational drug design. Such methods are useful for screening compounds having an effect on E. coli infection.

For therapeutic uses, the compositions or agents identified using the methods disclosed herein may be administered systemically, for example, formulated in a pharmaceutically-acceptable buffer such as physiological saline. Treatment may be accomplished directly, e.g., by treating the animal (e.g., a human) with antagonists which disrupt, suppress, attenuate, or neutralize the biological events associated with a pathogenicity polypeptide. Preferable routes of administration include, for example, subcutaneous, intravenous, interperitoneally, intramuscular, or intradermal injections which provide continuous, sustained levels of the drug in the patient. Treatment of human patients or other animals will be carried out using a therapeutically effective amount of an anti-pathogenic agent in a physiologically-acceptable carrier. Suitable carriers and their formulation are described, for example, in Remington's Pharmaceutical Sciences by E. W. Martin. The amount of the anti-pathogenic agent (e.g., an antibiotic) to be administered varies depending upon the manner of administration, the age and body weight of the patient, and with the type of disease and extensiveness of the disease. Generally, amounts will be in the range of those used for other agents used in the treatment of other microbial diseases, although in certain instances lower amounts will be needed because of the increased specificity of the compound. A compound is administered at a dosage that inhibits microbial proliferation. For example, for systemic administration a compound is administered typically in the range of 0.1 ng-10 g/kg body weight.

Polypeptides according to the invention (such as any one described in Tables 2, 3, or 7) may be purified and isolated by methods known in the art. In particular, having identified a gene sequence, it will be possible to use recombinant techniques to express the gene in a suitable host. Active fragments and related molecules can be identified and may be useful in therapy and are formulated according to methods known in the art. For example, a peptide or its active fragment may be used as an antigenic determinant in a vaccine, to elicit an immune response.

There are at least two critical properties of pathogenic antigens (e.g., a bacterial antigen) in determining their efficacy as a vaccine: availability and immunogenicity. An antigen must be expressed by the disease-causing agent and made available to the immune system in order for it to be a suitable target for an immune response. Furthermore, when available to the immune system, it must be sufficiently immunogenic to induce immunity. The screens described herein, by their very design, have exclusively identified E. coli O157 antigens that possess these properties. Therefore any of the above listed proteins would be suitable for inducing an immune response to E. coli O157 in mammals.

Proteins identified in either the IVIAT or PELS screen are suitable as vaccines for use in all mammals, including both humans and cattle. It is well known in the art that calves are suitable models for testing the efficacy of vaccines against bacterial pathogens (Skinner et al., Infect Immun. 2005 July; 73(7): 4441-4444). Methods are well known in the art to confirm whether any of the proteins identified in either of the above described assays is immunogenic in a particular mammal. Briefly, the mammal is injected with the protein and a pharmecutically acceptable carrier. After a sufficient duration, blood is drawn from the mammal and assayed for antibodies to the selected protein.

Different types of vaccines can be developed according to standard procedures known in the art. For example, a vaccine may be peptide-based, nucleic acid-based, bacterial- or viral-based vaccines. A vaccine formulation containing at least one polypeptide or nucleic acid of the invention may contain a variety of other components, including stabilizers. The vaccine also optionally includes or is co-administered with one or more suitable adjuvants, such as a mucosal adjuvant. The mucosal adjuvant may be any known in the art appropriate for human use (e.g., cholera toxin (CT), enterotoxigenic E. coli heat-labile toxin (LT), or a derivative, subunit, or fragment of CT or LT which retains adjuvanticity). The mucosal adjuvant is co-administered with the polypeptide vaccine in an amount effective to induce or enhance a mucosal immune response, particularly a humoral and/or a mucosal immune response. The ratio of adjuvant to the polypeptide vaccine may be determined by standard methods by one skilled in the art.

They may also be used in the preparation of antibodies, for passive immunization, or diagnostic applications. Suitable antibodies include monoclonal antibodies, or fragments thereof, including single-chain Fv fragments. Methods for the preparation of antibodies will be apparent to those skilled in the art.

Active fragments are those that retain a biological function of the peptide or which generate antibodies that are specific for that peptide. For example, when used to elicit an immune response, the fragment will be of sufficient size, such that antibodies generated from the fragment will discriminate between that peptide and other peptides of the bacterial microorganism. Typically, the fragment will be at least 30 nucleotides (10 amino acids) in size, preferably 60 nucleotides (20 amino acids) and most preferably greater than 90 nucleotides (30 amino acids) in size or according to the sizes described herein. Such fragments are formulated according to standard methods known in the art.

### Diagnostics

The hybridization of any of the nucleic acids molecules identified in Tables 2, 3, or 7 or both are useful in determining the bacterial strain profile according to methods known in the art. Such nucleic acids may be used as targets in a microarray. The microarray is used to assay the bacterial strain typing profile

TABLE 1
Ivi-proteins with a reported role in O157 pathogenesis.

| | Insert | | | | | |
|---|---|---|---|---|---|---|
| | localization | | | | No. of reactive sera³ | |
| IVIAT | on EDL933 | | | Contig/plasmid | HUS sera | Control |
| clone | genome | Gene/Protein/Function¹ | Bacterial cell localization² | accession number | (4) | serum (1) |
| H-310 | OI # 148 | eae/intimin/attaching | Outer membrane | AE005595 | 4 | 0 |
| | | and effacing | | | | |
| H-13, 75 | pO157 | msbB2/MsbB/lipid A | Inner membrane | AF074613 | 4 | 0 |
| | | acyltransferase | | | | |
| H-124 | pO157 | L7031/probable toxR- | Inner membrane | AF074613 | 4 | 0 |
| | | regulated lipoprotein | | | | |
| | | TagA | | | | |
| H-314 | Backbone | qseA/transcriptional | Cytoplasm | AE005552 | 4 | 0 |
| | | regulator, LysR type; | | | | |
| | | quorum sensing | | | | |
| | | regulator A | | | | |

¹Putative function of hypothetical proteins determined from the cluster of orthologous groups (COGs) when available.
²Bacterial cell localization of proteins predicted by the PSORT/PSORT-B program.
³Sera from (4) patients with HUS, and (1) healthy person were tested individually.

TABLE 2
Backbone encoded O157 ivi-proteins.

| Amino | Nucleic | | | | Contig | No. of reactive sera³ | |
|---|---|---|---|---|---|---|---|
| Acid SEQ | Acid SEQ | IVIAT | Gene/Protein/Specific | Bacterial cell | Accession | HUS | Control. |
| ID NO: | ID NO: | clone | function¹ | localization² | NO. | sera (4) | sera (1) |

Macromolecule Synthesis:

| | | | | | | | |
|---|---|---|---|---|---|---|---|
| | | H-5, 20 | topA/DNA topoisomerase | Cytoplasm | AE005379 | 4 | 0 |
| | | | type I, omega protein/DNA | | | | |
| | | | replication, repair, | | | | |
| | | | restriction/modification | | | | |
| | | H-9 | dsbA/protein disulfide | Periplasm | AE005616 | 4 | 0 |
| | | | isomerase I essential for | | | | |
| | | | cytochrome C synthesis and | | | | |
| | | | formate-dependent | | | | |
| | | | reduction/protein | | | | |
| | | | translation and f | | | | |
| | | | modification | | | | |
| | | H-22, 23 | crcB/putative protein | Inner | AE005242 | 4 | 0 |
| | | | involved in chromosome | membrane | | | |
| | | | condensation | | | | |
| | | H-37 | purK/ | Cytoplasm | AE005233 | 4 | 0 |
| | | | phosphoribosylaminoimidazole | | | | |
| | | | carboxylase = AIR | | | | |
| | | | carboxylase, CO₂fixing | | | | |
| | | | subunit/purine | | | | |
| | | | ribonucleotide biosynthesis | | | | |
| | | H-40 | purD/ | Cytoplasm | AE005632 | 4 | 0 |
| | | | phosphoribosylglycinamide | | | | |
| | | | synthetase = GAR synthetase/ | | | | |
| | | | purine ribonucleotide | | | | |
| | | | biosynthesis | | | | |
| 1 | 2 | H-42 | apbA/thiamine | Inner | AE005221 | 4 | 0 |
| | | | biosynthesis, alternate | membrane | | | |
| | | | pyrimidine biosynthesis | | | | |
| | | H-110 | recR/DNA replication, | Cytoplasm | AE005487 | 4 | 0 |
| | | | repair, | | | | |
| | | | restriction/modification | | | | |
| | | H-121 | cysD/ATP: sulfate | Cytoplasm | AE005502 | 4 | 0 |
| | | | adenylytransferase, subunit 2 | | | | |
| | | H-145, 183 | glgP/glycogen | Cytoplasm | AE005566 | 4 | 0 |
| | | | phosphorylase/ | | | | |
| | | | polysaccharide modification | | | | |
| | | H-211 | mutH/methyl-directed | Cytoplasm | AE005512 | 4 | 1 |
| | | | mismatch repair/DNA | | | | |
| | | | replication, repair, | | | | |
| | | | restriction/modification | | | | |
| | | H-230 | Z2851/putative Rad3- | Cytoplasm | AE005403 | 1 | 0 |
| | | | related DNA helicase | | | | |
| 3 | 4 | H-245, 260 | dfp/flavoprotein affecting | Inner | AB005591 | 4 | 0 |
| | | | synthesis of DNA and | membrane | | | |
| | | | pantothenate metabolism/ | | | | |
| | | | DNA replication, repair, | | | | |
| | | | restriction/modification | | | | |
| | | H-313 | mutS/methyl-directed | Cytoplasm | AE005501 | 4 | 0 |
| | | | mismatch repair/DNA | | | | |
| | | | replication, repair, | | | | |
| | | | restriction/modification | | | | |

Small Molecule Synthesis:

| | | | | | | | |
|---|---|---|---|---|---|---|---|
| 5 | 6 | H-11 | ggt/gamma- | Periplasm | AE005568 | 4 | 1 |
| | | | glutamyltranspeptidase/ | | | | |
| | | | biosynthesis of cofactors, | | | | |
| | | | carriers: thioredoxin, | | | | |
| | | | glutaredoxin, glutathione | | | | |
| | | H-60 | Z3637/putative thiamine | Cytoplasm | AE005468 | 4 | 0 |
| | | | pyrophosphate requiring | | | | |
| | | | enzyme - isoleucine, | | | | |
| | | | leucine, valine biosynthesis | | | | |
| | | H-71 | dapD/2,3,4,5- | Cytoplasm | AE005192 | 2 | 1 |
| | | | tetrahydropyridine-2- | | | | |
| | | | carboxylate N-succinyl | | | | |
| | | | transferase/amino acid | | | | |
| | | | biosynthesis: lysine | | | | |
| | | H-116, 137 | glyA/serine | Inner | AE005485 | 4 | 0 |
| | | | hydroxymethyltransferase/ | membrane | | | |
| | | | amino acid biosynthesis: | | | | |
| | | | glycine | | | | |
| | | H-117 | accC/acetyl CoA | Cytoplasm | AE005553 | 4 | 0 |
| | | | carboxylase, biotin | | | | |
| | | | carboxylase subunit/fatty | | | | |
| | | | acid and phosphatidic acid | | | | |
| | | | biosynthesis | | | | |
| | | H-131, 133 | hemK/putative | Cytoplasm | AE005338 | 4 | 0 |
| | | | protoporphyrinogen oxidase/ | | | | |
| | | | biosynthesis of cofactors: | | | | |
| | | | heme, porphyrin | | | | |
| | | H-132 | alaS/alanyl-tRNA | Inner | AE005498 | 4 | 0 |
| | | | synthetase/tRNA | membrane | | | |
| | | | modification | | | | |
| | | H-164 | folE/GTP cyclohydrase/ | Cytoplasm | AE005447 | 4 | 0 |
| | | | biosynthesis of cofactors, | | | | |
| | | | carriers:folic acid | | | | |
| | | H-174 | nadE/NAD synthetase/ | Cytoplasm | AE005397 | 4 | 0 |
| | | | biosynthesis of cofactors, | | | | |
| | | | carriers: pyridine | | | | |
| | | H-177 | hisS/histidine-tRNA | Inner | AE005480 | 4 | 1 |
| | | | synthetase/tRNA | membrane | | | |
| | | | modification | | | | |
| 7 | 8 | H-194 | Z2789/putative thiosulfate | Periplasm | AE005398 | 4 | 0 |
| | | | sulfur transferase | | | | |
| 9 | 10 | H-227 | cstC/acetylornithine delta- | Inner | AE005397 | 4 | 0 |
| | | | aminotransferase/amino | membrane | | | |
| | | | acid biosynthesis: arginine | | | | |
| | | H-267 | lysS/lysine-tRNA | Cytoplasm | AE005519 | 4 | 0 |
| | | | synthetase/tRNA | | | | |
| | | | modification | | | | |
| 11 | 12 | H-279, 280, | proB/gamma-glutamate | Inner | AE005202 | 4 | 0 |
| | | 282, 283, | kinase/amino acid | membrane | | | |
| | | 308, 312 | biosynthesis: praline | | | | |

Macromolecule Degradation:

| | | | | | | | |
|---|---|---|---|---|---|---|---|
| | | H-79 | hycI/protease/process C- | Cytoplasm | AE005500 | 2 | 0 |
| | | | terminal end of the large | | | | |
| | | | subunit of hydrogenase 3 | | | | |
| | | H-99 | Z5946/putative restriction | Cytoplasm | AE005666 | 2 | 0 |
| | | | endonuclease S subunits | | | | |
| | | H-100, 253 | Z3649/putative cellulase M | Cytoplasm | AE005469 | 4 | 0 |
| | | | and related protein | | | | |
| | | H-140, 154, | dcp/dipeptidyl | Cytoplasm | AE005351 | 4 | 0 |
| | | 215, 218, | carboxypeptidase II/ | | | | |
| | | 219, 222 | degradation of proteins, | | | | |
| | | | peptides, glycopeptides | | | | |
| 13 | 14 | H-173 | Z1305/putative ATP- | Inner | AE005285 | 2 | 0 |
| | | | dependent protease | membrane | | | |
| | | H-185 | malS/alpha-amylase/ | Periplasm | AE005584 | 4 | 0 |
| | | | degradation of | | | | |
| | | | polysaccharides | | | | |
| | | H-188 | uvrB/excision nuclease | Cytoplasm | AE005259 | 4 | 0 |
| | | | subunit B/degradation of | | | | |
| | | | DNA | | | | |
| | | H-236 | Z2427/putative metal- | Cytoplasm | AE005372 | 4 | 0 |
| | | | dependent | | | | |
| | | | amidase/aminoacylase/carboxypeptidase | | | | |
| 15 | 16 | H-242 | exo/5′-3′ exonuclease/ | Inner | AE005508 | 4 | 0 |
| | | | degradation of DNA | membrane | | | |
| 17 | 18 | H-304, 307, | endA/endonuclease I/ | Periplasm | AE005525 | 4 | 0 |
| | | 309 | degradation of DNA | | | | |

Small molecule Degradation:

| | | | | | | | |
|---|---|---|---|---|---|---|---|
| | | H-7 | FadA/thiolaase I/ | Cytoplasm | AE005615 | 4 | 0 |
| | | | degradation of fatty acids | | | | |
| | | H-8 | rnt/ribonuclease T/ | Cytoplasm | AE005388 | 4 | 0 |
| | | | degrades tRNA | | | | |
| | | H-46, 67 | xylA/D-xylose isomerase/ | Cytoplasm | AE005583 | 4 | 0 |
| | | | degradation of carbon | | | | |
| | | | compounds | | | | |
| | | H-58 | Z0666/putative | Cytoplasm | AE005232 | 4 | 0 |
| | | | dihydroorotase and related | | | | |
| | | | cyclic amidohydrolases | | | | |
| 19 | 20 | H-74 | hflB/integral membrane | Inner | AE005546 | 4 | 0 |
| | | | peptidase/degrades sigma | membrane | | | |
| | | | 32 | | | | |
| | | H-91, 93 | malY/enzyme that blocks | Cytoplasm | AE005386 | 4 | 0 |
| | | | biosynthesis or degrades | | | | |
| | | | endogenous Mal inducer, | | | | |
| | | | probably aminotransferase/ | | | | |
| | | | degradation of carbon | | | | |
| | | | compounds | | | | |
| 21 | 22 | H-104 | galK/galactokinase/ | Inner | AE005253 | 4 | 0 |
| | | | degradation of carbon | membrane | | | |
| | | | compounds | | | | |
| | | H-166 | celF/phosphor-beta- | Cytoplasm | AE005396 | 4 | 0 |
| | | | glucosidase/degradation of | | | | |
| | | | carbon compounds | | | | |
| 23 | 24 | H-175 | treF/trehalase/ | Inner | AE005577 | 4 | 0 |
| | | | degradation of carbon | membrane | | | |
| | | | compounds | | | | |
| | | H-178, 241 | YciA/putative acyl CoA | Cytoplasm | AE005342 | 2 | 0 |
| | | | hydrolase | | | | |
| 25 | 26 | H-301 | malZ/maltodextrin | Inner | AE005219 | 4 | 0 |
| | | | glucosidase/degradation of | membrane | | | |
| | | | carbon compounds | | | | |

Energy/Metabolism:

| | | | | | | | |
|---|---|---|---|---|---|---|---|
| | | H-3 | yhaF/putative 2,4, | Cytoplasm | AE005541 | 4 | 0 |
| | | | dihydroxyhept-2-ene- | | | | |
| | | | 1,7dioic | | | | |
| | | | acid aldolase | | | | |
| 27 | 28 | H-16 | Z2579/putative DMSO | Inner | AE005382 | 4 | 0 |
| | | | reductase | membrane | | | |
| | | H-19 | Z2779/putative | Cytoplasm | AE005397 | 4 | 0 |
| | | | arginine/ornithine N- | | | | |
| | | | succinyl transferase beta | | | | |
| | | | subunit | | | | |
| | | H-29 | fba/fructose-bisphosphate | Cytoplasm | AE005522 | 4 | 0 |
| | | | aldolase, class II/glycolysis | | | | |
| | | H-35 | Z2723/putative | Cytoplasm | AE005392 | 4 | 0 |
| | | | oxidoreductase | | | | |
| | | H-39 | hycG/hydrogenase/carbon | Cytoplasm | AE005500 | 4 | 0 |
| | | | fermentation | | | | |
| | | H-49, 50, | adhP/alcohol | Cytoplasm | AE005357 | 4 | 0 |
| | | 52, 53, 134 | dehydrogenase/anaerobic | | | | |
| | | | respiration | | | | |
| | | H-54 | ycaH/putative | Cytoplasm | AE005281 | 4 | 1 |
| | | | tetraacyldisaccharide-1-P4′- | | | | |
| | | | kinase | | | | |
| | | H-72 | narW/cryptic nitrate | Cytoplasm | AE005359 | 4 | 0 |
| | | | reductase 2, delta subunit/ | | | | |
| | | | anaerobic respiration | | | | |
| 29 | 30 | H-85 | dmsC/dimethyl sulfoxide | Inner | AE005279 | 4 | 0 |
| | | | reductase, subunit C/ | membrane | | | |
| | | | anaerobic respiration | | | | |
| 31 | 32 | H-88 | ccmH/possible subunit of | Inner | AE005452 | 4 | 1 |
| | | | heme lyase/electron | membrane | | | |
| | | | transport | | | | |
| 33 | 34 | H-103, 165 | ygjL/putative NADPH | Inner | AE005538 | 4 | 0 |
| | | | dehydrogenase | membrane | | | |
| 35 | 36 | H-105 | nrfE/formate dependent | Inner | AE005640 | 4 | 0 |
| | | | nitrite reductase/anaerobic | membrane | | | |
| | | | respiration | | | | |
| 37 | 38 | H-108 | phoA/alkaline phosphatase/ | Periplasm | AE005217 | 4 | 1 |
| | | | central intermediary | | | | |
| | | | metabolism | | | | |
| | | H-111 | yhdJ/putative | Cytoplasm | AE005554 | 4 | 0 |
| | | | methyltransferase | | | | |
| 39 | 40 | H-114, 142 | visC/putative 2-polyprenyl- | Inner | AE005521 | 4 | 0 |
| | | | 6-methoxyphenol | membrane | | | |
| | | | hydorxylase and related | | | | |
| | | | FAD-dependent | | | | |
| | | | oxidoreductases | | | | |
| 41 | 42 | H-118 | citF/citrate lyase alpha | Inner | AE005241 | 4 | 0 |
| | | | chain/central intermediary | membrane | | | |
| | | | metabolism | | | | |
| 43 | 44 | H-122 | yleB/putative 2- | Inner | AE005245 | 4 | 0 |
| | | | polyprenyl-6- | membrane | | | |
| | | | methoxyphenol hydorxylase | | | | |
| | | | and related FAD-dependent | | | | |
| | | | oxidoreductases | | | | |
| 45 | 46 | H-138 | citE/citrate lyase beta | Inner | AE005241 | 4 | 0 |
| | | | chain/central intermediary | membrane | | | |
| | | | metabolism | | | | |
| | | H-147 | ygfZ/putative | Cytoplasm | AE005520 | 4 | 0 |
| | | | aminomethyltransferase | | | | |
| | | | related to GcvT | | | | |
| | | H-149 | nrdB/ribonucleoside | Cytoplasm | AE005456 | 4 | 0 |
| | | | diphosphage reductase, beta | | | | |
| | | | sbunit, B2/central | | | | |
| | | | intermediary metabolism | | | | |
| 47 | 48 | H-153 | agaV/N- | Inner | AE005542 | 4 | 0 |
| | | | acetylgalactosamine- | membrane | | | |
| | | | specific IIB component 2 | | | | |
| | | | (EIIB-AGA)/central | | | | |
| | | | intermediary metabolism | | | | |
| 49 | 50 | H-160, 264 | Z4220/putative | Inner | AE005518 | 4 | 0 |
| | | | dehydrogenase | membrane | | | |
| 51 | 52 | H-167 | nuoL/NADH | Inner | AE005460 | 4 | 1 |
| | | | dehydrogenase I chain L/ | membrane | | | |
| | | | anaerobic respiration | | | | |
| 53 | 54 | H-171, 306 | hybD/probable processing | Inner | AE005529 | 4 | 0 |
| | | | element for hydrogenase-2/ | membrane | | | |
| | | | anaerobic respiration | | | | |
| 55 | 56 | H-186 | nuoM/NADH | Inner | AE005459 | 4 | 1 |
| | | | dehydrogenase I chain M/ | membrane | | | |
| | | | anaerobic respiration | | | | |
| 57 | 58 | H-205 | Z3775/putative | Outer | AE005480 | 4 | 0 |
| | | | dehydrogenase | membrane | | | |
| | | H-216 | Z4018/putative flavodoxin | Cytoplasm | AE005499 | 4 | 0 |
| | | H-217, 221, | Z5951/putative GTPase | Cytoplasm | AE005666 | 4 | 0 |
| | | 223 | (G3E family) | | | | |
| | | H-224 | yhjL/putative | Cytoplasm | AE005579 | 4 | 0 |
| | | | oxidoreductase | | | | |
| | | H-233, 288 | narY/cryptic nitrate | Cytoplasm | AE005359 | 4 | 0 |
| | | | reductase 2, beta subunit/ | | | | |
| | | | anaerobic respiration | | | | |
| 59 | 60 | H-237 | solA/putative sarcosine | Periplasm | AE005316 | 2 | 0 |
| | | | oxidase-like protein | | | | |
| | | H-239 | yidS/putative | Cytoplasm | AE005600 | 4 | 0 |
| | | | dehydrogenase | | | | |
| | | | (flavoprotein) | | | | |
| 61 | 62 | H-244 | ugd/UDP-glucose-6- | Inner | AE005428 | 4 | 0 |
| | | | dehydrogenase/central | membrane | | | |
| | | | intermediary metabolism | | | | |
| 63 | 64 | H-254 | yliI/putative | Periplasm | AE005265 | 4 | 0 |
| | | | dehydrogenase | | | | |
| | | H-258 | Z3401/putative | Inner | AE005446 | 4 | 0 |
| | | | oxidoreductase | membrane | | | |
| | | H-270, 276 | epd/D-erythrose-4- | Cytoplasm | AE005523 | 4 | 0 |
| | | | phosphate dehydrogenase/ | | | | |
| | | | central intermediary | | | | |
| | | | metabolism | | | | |
| | | H-277 | ybjT/putative dTDP- | Inner | AE005268 | 4 | 0 |
| | | | glucose enzyme | membrane | | | |
| | | H-287 | yraL/putative | Cytoplasm | AE005543 | 4 | 0 |
| | | | methyltransferase | | | | |
| | | H-292 | Z3734/putative | Inner | AE005477 | 4 | 0 |
| | | | metalloprotease | membrane | | | |
| | | H-295 | yeaA/putative peptide | Cytoplasm | AE005401 | 4 | 0 |
| | | | methionine sulfoxide | | | | |
| | | | reductase | | | | |
| | | H-300 | wecB/UDP-N-acetyl | Cytoplasm | AE005610 | 4 | 0 |
| | | | glucosamine-2-epimerase/ | | | | |
| | | | central intermediary | | | | |
| | | | metabolism | | | | |
| | | H-305 | tktA/transketolase 1 | Cytoplasm | AE005524 | 4 | 0 |
| | | | isoenzyme/central | | | | |
| | | | intermediary metabolism | | | | |
| | | H-315 | pflD/formate acetyl- | Cytoplasm | AE005626 | 4 | 0 |
| | | | transferase 2/anaerobic | | | | |
| | | | respiration | | | | |

Regulatory:

| | | | | | | | |
|---|---|---|---|---|---|---|---|
| 65 | 66 | H-1 | basS/BasS/sensor protein | Inner | AE005644 | 4 | 0 |
| | | | for BasR involved in | membrane | | | |
| | | | macromolecule synthesis | | | | |
| | | H-14 | phoQ/pho Q/sensor | Inner | AE005328 | 4 | 0 |
| | | | protein; global regulation | membrane | | | |
| | | H-24 | mopB/GroES chaperone/ | Cytoplasm | AE005648 | 2 | 0 |
| | | | folding and ushering | | | | |
| | | | proteins | | | | |
| 67 | 68 | H-27 | pqiA/paraquat-inducible | Inner | AE005285 | 4 | 1 |
| | | | protein A/not characterized | membrane | | | |
| | | H-45, 87, | xylR/XylR/regulates the | Cytoplasm | AE005583 | 4 | 0 |
| | | 290, 293 | xyl operon involved in | | | | |
| | | | xylose utilization | | | | |
| | | H-81, 83 | ydeW/putative | Inner | AE005353 | 4 | 0 |
| | | | transcriptional regulator | membrane | | | |
| | | H-82 | glnG/GlnG/response | Cytoplasm | AE005617 | 4 | 0 |
| | | | regulator for gln operon | | | | |
| | | | involved in glutamine | | | | |
| | | | biosynthesis; interacts with | | | | |
| | | | sensor glnL | | | | |
| 69 | 70 | H-86 | rcsF/RcsF/regulates the | Outer | AE005195 | 4 | 0 |
| | | | rcs regulon involved in | membrane | | | |
| | | | colanic acid synthesis; | | | | |
| | | | interacts with rcsB | | | | |
| | | H-94 | yieP/putative | Cytoplasm | AE005607 | 4 | 1 |
| | | | transcriptional regulator | | | | |
| 71 | 72 | H-120, 148 | glnL/GlnL/histidine | Inner | AE0055617 | 4 | 1 |
| | | | protein kinase sensor for the | membrane | | | |
| | | | GlnG regulator; glutamine | | | | |
| | | | biosynthesis | | | | |
| | | H-127 | yifB/putative 2-component | Periplasm | AE005608 | 4 | 1 |
| | | | regulator | | | | |
| | | H-141 | uidR/UidR/regulates the | Cytoplasm | AE005385 | 4 | 0 |
| | | | uid operon involved in beta- | | | | |
| | | | glucuronidase synthesis | | | | |
| | | H-159 | prpR/PrpR/regulates the | Cytoplasm | AE005212 | 4 | 1 |
| | | | prp operon involved in | | | | |
| | | | propionate catabolism | | | | |
| | | H-192 | yfeR/putative | Cytoplasm | AE005471 | 4 | 0 |
| | | | transcriptional regulator, | | | | |
| | | | LysR type | | | | |
| | | H-193 | uvrY/putative 2-component | Cytoplasm | AE005414 | 4 | 0 |
| | | | regulator | | | | |
| | | H-195 | ybcZ/putative 2-component | Inner | AE005236 | 4 | 0 |
| | | | sensor | membrane | | | |
| | | H-201 | yeiL/putative | Inner | AE005448 | 4 | 0 |
| | | | transcriptional regulator | membrane | | | |
| | | H-256 | yifA/putative | Cytoplasm | AE005607 | 4 | 0 |
| | | | transcriptional regulator | | | | |
| | | H-262 | fucR/FucR/positive | Cytoplasm | AE005508 | 4 | 0 |
| | | | regulator of the fuc operon | | | | |
| | | H-274 | fliS/Fli S/regulates | Cytoplasm | AE005415 | 4 | 0 |
| | | | flagellar biosynthesis | | | | |
| 73 | 74 | H-278 | hydH/Hyd H/sensor | Inner | AE005632 | 4 | 0 |
| | | | kinase for HydG, | membrane | | | |
| | | | hydrogenase 3 activity | | | | |
| 75 | 76 | H-297 | narX/NarX/nitrate/nitrate | Inner | AE005339 | 2 | 0 |
| | | | sensor, histidine protein | membrane | | | |
| | | | kinase acts on NarL | | | | |
| | | | regulator | | | | |
| | | H-303 | yhjC/putative | Inner | AE005577 | 4 | 0 |
| | | | transcriptional regulator, | membrane | | | |
| | | | LysR type | | | | |
| | | H-311 | Z2724/putative ARAC- | Cytoplasm | AE005392 | 4 | 0 |
| | | | type regulator | | | | |

Transport:

| | | | | | | | |
|---|---|---|---|---|---|---|---|
| 77 | 78 | H-4, 289 | ascF/AscF; PTS system | Inner | AE005500 | 4 | 0 |
| | | | enzyme II/transport of | membrane | | | |
| | | | small molecules: arbutin, | | | | |
| | | | salicin, cellobiose | | | | |
| | | H-10 | ybhS/putative ABC-type | Inner | AE005260 | 4 | 1 |
| | | | multidrug transport system, | membrane | | | |
| | | | permease component | | | | |
| 79 | 80 | H-12 | wzxC/putative export | Inner | AE005430 | 4 | 0 |
| | | | protein; protein, peptide | membrane | | | |
| | | | secretion | | | | |
| | | H-15 | FlhB/flhB/export of | Inner | AE005410 | 4 | 0 |
| | | | flagellar proteins | membrane | | | |
| | | H-28 | yohM/putative ABC-type | Inner | AE005437 | 4 | 0 |
| | | | uncharacterized transport | membrane | | | |
| | | | system, permease | | | | |
| | | | component | | | | |
| 81 | 82 | H-31 | tdcC/TdcC; anaerobically | Inner | AE005540 | 4 | 0 |
| | | | induced permease/transport | membrane | | | |
| | | | of amino acids: L-threonine, | | | | |
| | | | L-serine | | | | |
| 83 | 84 | H-34 | malF/MalF; permease/ | Inner | AE005636 | 4 | 0 |
| | | | transport of small | membrane | | | |
| | | | molecules: maltose | | | | |
| | | H-44, 200, | yegT/putative nucleoside | Inner | AE005436 | 4 | 0 |
| | | 213, 250, | permease | membrane | | | |
| | | 251, 286 | | | | | |
| 85 | 86 | H-47, 66, | ycbQ/putative chaperone | Outer | AE005284 | 4 | 0 |
| | | 266 | | membrane | | | |
| | | H-59 | yedE/putative transport | Inner | AE005415 | 4 | 0 |
| | | | system permease protein | membrane | | | |
| | | H-73 | Z4150/putative transport | Inner | AE005512 | 4 | 0 |
| | | | protein | membrane | | | |
| | | H-76 | yhfC/putative transport | Inner | AE005559 | 4 | 0 |
| | | | protein | membrane | | | |
| 87 | 88 | H-92 | yaeM/putative ATP- | Inner | AE005193 | 4 | 0 |
| | | | binding component of | membrane | | | |
| | | | transport system | | | | |
| | | H-101 | Z2654/putative chaperone | Cytoplasm | AE005387 | 4 | 0 |
| | | | distantly related to HSP70- | | | | |
| | | | fold metalloprotease | | | | |
| | | H-109, 168, | livG/LivG; ATP-binding | Cytoplasm | AE005569 | 4 | 0 |
| | | 169, 172, | component of high affinity | | | | |
| | | 182, 184 | branched-chain amino acid | | | | |
| | | | transport system/transport | | | | |
| | | | of amino acids, amines | | | | |
| | | H-115 | ugpC/UgpC; ATP-binding | Cytoplasm | AE005568 | 4 | 0 |
| | | | component of glycerol-3- | | | | |
| | | | phopshate transport system/ | | | | |
| | | | transport of small | | | | |
| | | | molecules: carbohydrates, | | | | |
| | | | organic acids, alcohols | | | | |
| 89 | 90 | H-125, 275 | ycdG/putative | Inner | AE005300 | 4 | 0 |
| | | | xanthine/uracil permease | membrane | | | |
| 91 | 92 | H-126 | kefC/KefC/transport of | Inner | AE005181 | 4 | 0 |
| | | | cations: K+ efflux | membrane | | | |
| | | | antiporter, glutathione | | | | |
| | | | regulated | | | | |
| 93 | 94 | H-143 | Z3262/putative ADP- | Inner | AE005436 | 4 | 0 |
| | | | ribosylglycohydrolase | membrane | | | |
| 95 | 96 | H-163 | yhfM/putative amino | Inner | AE005560 | 2 | 0 |
| | | | acid/amine transport protein | membrane | | | |
| | | H-176 | Z5178/putative PTS | Inner | AE005600 | 4 | 0 |
| | | | component; transport of | Membrane | | | |
| | | | carbohydrates, organic | | | | |
| | | | acids, alcohols | | | | |
| 97 | 98 | H-187 | wzb/putative tyrosine | Inner | AE005432 | 4 | 0 |
| | | | phosphatase | membrane | | | |
| 99 | 100 | H-197 | kgtP/KgtP; alpha- | Inner | AE005489 | 4 | 0 |
| | | | ketoglutarate permease/ | membrane | | | |
| | | | transport of small | | | | |
| | | | molecules: carbohydrates, | | | | |
| | | | organic acids, alcohols | | | | |
| | | H-202, 261 | fhuA/FhuA/transport of | Outer | AE005191 | 4 | 0 |
| | | | ferrichrome (Fe3+) and | membrane | | | |
| | | | antibiotics, acts as a | | | | |
| | | | receptor for phages and | | | | |
| | | | colicins | | | | |
| 101 | 102 | H-206 | nagE/NagE; PTS system | Inner | AE005246 | 4 | 0 |
| | | | N-acetylglucosamine | membrane | | | |
| | | | specific enzyme IIABC/ | | | | |
| | | | transport of amino acids, | | | | |
| | | | amines | | | | |
| | | H-225 | sgaB/putative PTS, | Cytoplasm | AE005652 | 4 | 0 |
| | | | galactitol-specific IIB | | | | |
| | | | component | | | | |
| | | H-226 | Z2786/putative ABC-type | Periplasm | AE005398 | 4 | 0 |
| | | | uncharacterized transport | | | | |
| | | | system, periplasmic | | | | |
| | | | component | | | | |
| | | H-228 | yaaU/putative transport | Inner | AE005181 | 4 | 0 |
| | | | protein | membrane | | | |
| | | H-240 | cycA/CycA; permease/ | Inner | AE005653 | 4 | 0 |
| | | | transport of amino acids and | membrane | | | |
| | | | amines: D-alanine, D-serine, | | | | |
| | | | glycine | | | | |
| 103 | 104 | H-252 | oppB/OppB; oligopeptide | Inner | AE005342 | 4 | 0 |
| | | | transport permease protein/ | membrane | | | |
| | | | transport of large molecules: | | | | |
| | | | proteins, peptides | | | | |
| | | H-271 | Z5839/putative ATP- | Inner | AE005655 | 4 | 0 |
| | | | binding component of ABC- | membrane | | | |
| | | | type transport system | | | | |
| | | H-273 | Z3589/putative | Cytoplasm | AE005464 | 4 | 0 |
| | | | transporting ATPase | | | | |
| 105 | 106 | H-296 | araG/AraG; ATP-binding | Inner | AE005411 | 2 | 1 |
| | | | component of high affinity | membrane | | | |
| | | | L-arabinose transport | | | | |
| | | | system/transport of small | | | | |
| | | | molecules: L-arabinose | | | | |
| | | H-316 | Z2605/putative | Inner | AE005384 | 4 | 0 |
| | | | arginine/ornithine antiporter | membrane | | | |

Environmental adaptation (chemotaxis, motility, attachment, cell division):

| | | | | | | | |
|---|---|---|---|---|---|---|---|
| 107 | 108 | H-30 | trg/Trg; methyl-accepting | Inner | AE005363 | 4 | 0 |
| | | | chemotaxis protein III, | membrane | | | |
| | | | ribose sensor receptor/ | | | | |
| | | | regulator; chemotaxis and | | | | |
| | | | motility | | | | |
| 109 | 110 | H-36 | osmY/OsmY; | Periplasm | AE005668 | 4 | 0 |
| | | | hyperosmotically inducible | | | | |
| | | | protein/osmotic adaptation | | | | |
| | | H-41, 130, | yjbB/putative alpha helix | Inner | AE005634 | 4 | 0 |
| | | 152, 265 | protein | membrane | | | |
| | | H-55 | fisI/peptidoglycan | Inner | AE005185 | 4 | 0 |
| | | | synthetase/septum | membrane | | | |
| | | | formation | | | | |
| 111 | 112 | H-62, 63, | mepA/murein DD- | Periplasm | AE005464 | 4 | 0 |
| | | 64, 65 | endopeptidase/cell | | | | |
| | | | envelope synthesis | | | | |
| | | H-77 | sfmH/SfmH/fimbrial | Inner | AE005234 | 4 | 0 |
| | | | assembly | membrane | | | |
| | | H-95 | yaeH/putative structural | Cytoplasm | AE005192 | 4 | 0 |
| | | | protein | | | | |
| 113 | 114 | H-96 | cirA/CirA/receptor for | Outer | AE005447 | 4 | 0 |
| | | | iron-regulated colicin I | membrane | | | |
| | | H-150 | yhjD/putative membrane | Inner | AE005577 | 4 | 1 |
| | | | protein | membrane | | | |
| | | H-203 | Z3481/putative membrane | Inner | AE005454 | 4 | 0 |
| | | | protein | membrane | | | |
| 115 | 116 | H-234 | mltB/membrane-bound | Outer | AE005498 | 3 | 0 |
| | | | lytic murein | membrane | | | |
| | | | transglycosylase B/murein | | | | |
| | | | sacculus synthesis | | | | |
| | | H-246 | slt/soluble lytic murein | Inner | AE005670 | 4 | 0 |
| | | | transglycosylase/murein | membrane | | | |
| | | | sacculus synthesis | | | | |
| 117 | 118 | H-268 | tsr/methyl accepting | Inner | AE005667 | 4 | 0 |
| | | | chemotaxis protein I, serine | membrane | | | |
| | | | sensor receptor/regulator, | | | | |
| | | | chemotaxis and motility | | | | |
| | | H-281 | mdaA/MdaA/modulator | Cytoplasm | AE005266 | 4 | 0 |
| | | | of drug activity A | | | | |
| | | H-284 | yibP/putative membrane | Inner | AE005588 | 4 | 0 |
| | | | protein | membrane | | | |
| | | H-302 | phoE/PhoE/outer | Outer | AE005202 | 4 | 0 |
| | | | membrane porin protein E | membrane | | | |

Phage related:

| | | | | | | | |
|---|---|---|---|---|---|---|---|
| 119 | 120 | H-209 | Z1782/unknown encoded | Inner | AE005323 | 4 | 0 |
| | | | within CP-933N | membrane | | | |
| | | H-238 | hflx/GTP-binding subunit | Cytoplasm | AE005650 | 2 | 0 |
| | | | of protease specific for | | | | |
| | | | lambda CII repressor/ | | | | |
| | | | degradation of proteins, | | | | |
| | | | peptides, glycopeptides | | | | |

Unknown:

| | | | | | | | |
|---|---|---|---|---|---|---|---|
| | | H-43, 208, | yfiH/uncharacterized | Cytoplasm | AE005490 | 4 | 0 |
| | | 291 | conserved protein | | | | |
| 121 | 122 | H-57 | ydbD/unknown | Periplasm | AE005365 | 4 | 0 |
| | | H-68, 69 | ychA/uncharacterized | Cytoplasm | AE005338 | 4 | 0 |
| | | | conserved protein | | | | |
| 123 | 124 | H-80 | yfbB/putative enzyme | Inner | AE005458 | 2 | 0 |
| | | | | membrane | | | |
| | | H-97 | ybfE/unknown | Cytoplasm | AE005246 | 4 | 1 |
| | | H-157 | yacC/unknown | Cytoplasm | AE005188 | 4 | 0 |
| 125 | 126 | H-170 | Z4888/unknown | Inner | AE005573 | 4 | 0 |
| | | | | membrane | | | |
| 127 | 128 | H-179 | yciI/unknown | Inner | AE005342 | 4 | 0 |
| | | | | membrane | | | |
| | | H-190 | yjjX/unknown | Cytoplasm | AE005670 | 2 | 0 |
| 129 | 130 | H-198 | yaaH/unknown | Inner | AE005177 | 4 | 0 |
| | | | | membrane | | | |
| 131 | 132 | H-212 | ybiM/unknown | Inner | AE005261 | 4 | 1 |
| | | | | membrane | | | |
| 133 | 134 | H-243 | Z2619/unknown | Outer | AE005385 | 4 | 0 |
| | | | | membrane | | | |

¹Based on homology to the sequenced, E. coli O157 strain EDL933 genome. Putative function of hypothetical proteins determined from the cluster of orthologous groups (COGs) when available.
²Bacterial cell localization of proteins predicted by the PSORT/PSORT-B program.
³Sera from (4) patients with HUS, and (1) healthy person were tested individually.

TABLE 3
O-island and pO157 encoded O157 ivi-proteins.

| | | | Insert | | | Contig/ | No. of reactive | |
|---|---|---|---|---|---|---|---|---|
| | Amino | Nucleic | locaction on | | | plasmid | sera³ | |
| IVIAT | Acid SEQ | Acid SED | EDL 933 | Gene/Protein/ | Bacterial cell | accession | HUS | Control |
| clone | ID NO: | ID NO: | genome | Function¹ | localization | No. | sera (4) | sera (1) |

Assigned function:

| | | | | | | |
|---|---|---|---|---|---|---|
| H-285 | OI # 84 | wbdP/glycosyl | Cytoplasm | AE005429 | 4 | 0 |
| | | transferase/cell surface | | | | |
| | | polysaccharides and | | | | |
| | | antigen synthesis | | | | |

Putative function:

| | | | | | | | | |
|---|---|---|---|---|---|---|---|---|
| H-6 | | | OI # 47 | Z1554/putative ABC- | Inner | AE005305 | 4 | 0 |
| | | | | transport, lipoprotein | membrane | | | |
| | | | | release, permease | | | | |
| | | | | component/cell wall | | | | |
| | | | | biogenesis | | | | |
| H-17 | | | OI # 28 | Z0634/putative | Inner | AE005229 | 4 | 0 |
| | | | | cytoplasmic membrane, | membrane | | | |
| | | | | ABC-type bacteriocin/ | | | | |
| | | | | lantibiotic exporter/ | | | | |
| | | | | defense mechanism | | | | |
| H-25 | | | OI # 108⁴ | Z3936/putative | Cytoplasm | AE005493 | 4 | 0 |
| | | | | transposase, IS30 family | | | | |
| H-33 | | | OI # 43 | Z1214/predicted | Cytoplasm | AE005276 | 4 | 0 |
| | | | | esterase of the alpha-beta | | | | |
| | | | | hydrolase superfamily | | | | |
| H-48, 51, | | | OI # 172 | Z5901/putative helicase | Cytoplasm | AE005661 | 4 | 0 |
| 89, 90 | | | | with a unique C-terminal | | | | |
| | | | | domain including metal- | | | | |
| | | | | binding cysteine cluster | | | | |
| H-78 | 135 | 136 | OI # 9 | Z0348/putative | Inner | AE005205 | 2 | 0 |
| | | | | arabinose efflux | membrane | | | |
| | | | | permease/carbohydrate | | | | |
| | | | | transport | | | | |
| H-84 | | | OI# 17 | Z0419/putative ABC- | Inner | AE005211 | 4 | 0 |
| | | | | type transport permease | membrane | | | |
| H-98 | | | OI # 145 | waaD/putative | Periplasm | AE005590 | 4 | 0 |
| | | | | lipopolysaccharide | | | | |
| | | | | biosynthesis enzyme | | | | |
| | | | | (glycosyltransferase) | | | | |
| H-102 | | | OI # 138 | Z4856/putative acyl- | Inner | AE005571 | 4 | 0 |
| | | | | coenzyme A synthetase/ | membrane | | | |
| | | | | AMP-(fatty) acid ligase | | | | |
| H-113 | 137 | 138 | pO157 | L7004/putative | Cytoplasm- | AF074613 | 4 | 1 |
| | | | | hemolysin expression | Inner | | | |
| | | | | modulating protein - | membrane | | | |
| | | | | hypothetical protein | | | | |
| H-129 | | | OI # 157 | Z5415/predicted | Inner | AE005619 | 2 | 0 |
| | | | | permease | membrane | | | |
| H-135 | 139 | 140 | OI # 30 | Z0705/putative RhsA/ | Inner | AE005236 | 4 | 0 |
| | | | | Rhs family protein | membrane | | | |
| H-136 | | | pO157 | L7091/putative nickase | Cytoplasm | AF074613 | 4 | 0 |
| H-151 | | | OI # 40 | Z1089/putative | Cytoplasm | AE005267 | 4 | 1 |
| | | | | arylsufatase A | | | | |
| H-231 | | | OI # 172 | Z5888/predicted | Cytoplasm | AE005659 | 4 | 0 |
| | | | | transcriptional regulator | | | | |
| H-263 | | | OI # 139 | Z4882/uncharacterized | Cytoplasm | AE005572 | 4 | 0 |
| | | | | protein encoded in | | | | |
| | | | | hypervariable junctions | | | | |
| | | | | of pilus gene clusters | | | | |
| H-298 | 141 | 142 | pO157 | sopA/putative plasmid | Inner | AF074613 | 4 | 0 |
| | | | | partitioning protein A | membrane | | | |

Phage-related:

| | | | | | | | | |
|---|---|---|---|---|---|---|---|---|
| H-18 | | | OI # 52 | Z1930/putative | Inner | AE005334 | 4 | 1 |
| | | | | hydrolase or | membrane | | | |
| | | | | acyltransferase | | | | |
| | | | | (alpha/beta hydrolase | | | | |
| | | | | family), encoded within | | | | |
| | | | | CP-933X | | | | |
| H-38, 56 | | | OI # 52 | Z1883/putative DNA | Cytoplasm | AE005330 | 4 | 0 |
| | | | | packaging protein | | | | |
| | | | | encoded within CP-933X | | | | |
| H-70 | | | OI # 36 | Z0964/putative DNA | Cytoplasm | AE005256 | 4 | 1 |
| | | | | packaging protein | | | | |
| | | | | encoded within CP-933K | | | | |
| H-106 | | | OI # 45⁴ | Z1433/unknown | Cytoplasm | AE005295 | 4 | 0 |
| | | | | encoded within BP- | | | | |
| | | | | 933W | | | | |
| H-112 | | | OI # 52 | Z1882/putative phage | Cytoplasm | AE005330 | 4 | 0 |
| | | | | DNA packaging protein, | | | | |
| | | | | NU1 subunit of | | | | |
| | | | | terminase, encoded in | | | | |
| | | | | CP-933X | | | | |
| H-128 | | | OI # 52 | Z1888/putative capsid | Cytoplasm | AE005330 | 4 | 0 |
| | | | | protein of prophage CP- | | | | |
| | | | | 933X | | | | |
| H-139 | | | OI # 57 | Z2085/putative | Cytoplasm | AE005346 | 4 | 0 |
| | | | | exonuclease VIII | | | | |
| | | | | encoded within CP-933O | | | | |
| H-158 | | | OI # 93 | Z3327/unknown | Cytoplasm | AE005441 | 4 | 0 |
| | | | | encoded within CP-933V | | | | |
| H-191 | | | OI # 71⁴ | Z6060/putative Q anti- | Cytoplasm | AE006460 | 3 | 0 |
| | | | | terminator encoded | | | | |
| | | | | within CP-933P | | | | |
| H-196 | | | OI # 93⁴ | Z3334/unknown | Cytoplasm | AE005442 | 4 | 1 |
| | | | | encoded within CP-933V | | | | |
| H-247 | 143 | 144 | OI # 57⁴ | Z2100/unknown | Inner | AE005347 | 4 | 0 |
| | | | | encoded within CP-933O | membrane | | | |
| H-255, | 145 | 146 | OI # 36 | Z0975/putative tail | Inner | AE005257 | 4 | 0 |
| 257 | | | | component of CP-933K | membrane | | | |
| H-272 | 147 | 148 | OI # 79⁴ | Z3097/putative | Inner | AE005420 | 4 | 0 |
| | | | | peptidase - putative | membrane- | | | |
| | | | | head-tail connector | cytoplasm | | | |
| | | | | protein encoded within | | | | |
| | | | | CP-933U | | | | |

Unknown:

| | | | | | | | | |
|---|---|---|---|---|---|---|---|---|
| H-2 | 149 | 150 | OI # 172 | Z5897/unknown | Inner | AE005660 | 4 | 0 |
| | | | | | membrane | | | |
| H-21 | 151 | 152 | OI # 7 | Z0251/unknown | Cytoplasm | AE005198 | 4 | 0 |
| | | | | (uncharacterized protein | | | | |
| | | | | conserved in bacteria) | | | | |
| H-107 | 153 | 154 | OI # 142 | Z5002/unknown | Cytoplasm | AE005584 | 4 | 0 |
| | | | | (uncharacterized protein | | | | |
| | | | | conserved in bacteria) | | | | |
| H-119 | 155 | 156 | OI # 89 | Z3271/unknown | Inner | AE005436 | 4 | 0 |
| | | | | | membrane | | | |
| H-123 | 157 | 158 | OI # 89 | Z3269/unknown | Cytoplasm | AE005436 | 4 | 0 |
| H-146 | 159 | 160 | OI # 140 | Z4912/unknown | Cytoplasm | AE005576 | 3 | 0 |
| H-162 | 161 | 162 | OI # 102 | Z3616/unknown | Cytoplasm | AE005466 | 4 | 0 |
| H-269 | 163 | 164 | OI # 48⁴ | Z1606/unknown | Inner | AE005309 | 4 | 0 |
| | | | | | membrane | | | |

¹Putative function of hypothetical proteins determined from the cluster of orthologous groups (COGs) when available.
²Bacterial cell localization of proteins predicted by the PSORT/PSORT-B program.
³Sera from (4) patients with HUS, and (1) healthy person were tested individually.
⁴Homologous proteins also found on other O-islands.

TABLE 4
Ivi-proteins expressed from O-islands encoding putative virulence factors.

| O-island no. | Putative virulence factor | Ivi-protein/Function |
|---|---|---|
| OI#7 | Macrophage toxin and a chaperone | Z0251/Unknown |
| OI#28 | A RTX-toxin-like exoprotein and transport system | Z0634/Putative cytoplasmic membrane ABC-type |
| | | bacteriocin/lantibiotic exporter |
| OI#43 | Urease gene cluster | Z1214/A predicted estrase |
| OI#47 | Adhesin and polyketide or fatty acid biosynthesis system | Z1554/Putative ABC-type transporter |
| OI#48 | Urease gene cluster | Z1606/Unknown |
| OI#115 | TTSS and secreted proteins similar to Samonella-Shigella inv- | None identified |
| | spa host-cell invasion proteins | |
| OI#122 | Two toxins and a PagC-like virulence factor | None identified |
| OI#138 | Fatty acid biosynthesis system | Z4856/Putative acyl-coenzyme A synthetase/ |
| | | Fatty acid ligase |
| OI#148 | LEE proteins | Eae/γ-intimin/attaching and effacing |

TABLE 5
O157 backbone ivi-proteins expressed in vitro as identified by proteomic analysis.

| | | 6M Urea and 1% SDS | | Percent |
|---|---|---|---|---|
| | | denaturing solution¹ | | protein |
| No. | O157 ivi-protein | Run 1 | Run 2 | abundance |
| 1 | Z4279 transketolase 1 isozyme (tktA) | + | + | 0.49 |
| 2 | Z5747 GroES, 10 Kd chaperone binds to Hsp60 (mopB) | + | + | 0.32 |
| 3 | Z4263 fructose-bisphosphate aldolase, class II (fba) | + | + | 0.28 |
| 4 | Z4540 degrades sigma32, integral membrane peptidase, cell division (hflB) | + | − | 0.17 |
| 5 | Z4616 acetyl CoA carboxylase, biotin carboxylase subunit (accC) | + | − | 0.14 |
| 6 | Z4228 lysine tRNA synthetase, constitutive suppressor of ColE1 (lysS) | + | + | 0.13 |
| 7 | Z0176 2,3,4,5-tetrahydropyridine-2-carboxylate N-succinyltransferase (dapD) | + | + | 0.11 |
| 8 | Z3999 alanyl-tRNA synthetase (alaS) | + | + | 0.11 |
| 9 | Z2789 putative thiosulfate sulfur transferase | + | − | 0.10 |
| 10 | Z3827 serine hydroxymethyltransferase (glyA) | + | + | 0.09 |
| 11 | Z5977 hyperosmotically inducible periplasmic protein (osmY) | + | + | 0.08 |
| 12 | Z3777 histidine tRNA synthetase (hisS) | + | + | 0.07 |
| 13 | Z5955 methyl-accepting chemotaxis protein I, serine sensor receptor (tsr) | + | + | 0.06 |
| 14 | Z3491 ribonucleoside diphosphage reductase 1, beta subunit, B2 (nrdB) | + | + | 0.06 |
| 15 | Z4043 methyl-directed mismatch repair (mutS) | − | + | 0.05 |
| 16 | Z4478 orf, hypothetical protein (yhaF) | + | − | 0.05 |
| 17 | Z0998 DNA repair excision nuclease subunit B (uvrB) | − | + | 0.05 |
| 18 | Z5392 protein disulfide isomerase I, essential for cytochrome (dsbA) | + | + | 0.04 |

¹Two four hour runs were done using this denaturing condition. Each yielded more than 300 proteins.

TABLE 6
Previously reported adhesins of O157 and other pathogens identified by PELS.

| | Locus ID in O157 | | No. | Percent (%) | |
|---|---|---|---|---|---|
| | EDL 933/Sakai | Mass | peptide | protein | Contributes to |
| Protein | strains | (Kda) | hits | coverage | adherence in: |
| OmpA; outer membrane protein | Z1307/Ecs1041 | 38 | 42 | 43% | O157 |
| 3a (II*; G; d); adhesin | | | | | |
| Iha; adhesin; exogenous ferric | Z1178/Ecs1360 | 77 | 4 | 8% | O157, |
| siderophore receptor R4 | | | | | Uropathogenic E. coli |

TABLE 7
In vivo expressed O157 proteins identified by PELS in cattle.

| | | Region in the | | | | | | |
|---|---|---|---|---|---|---|---|---|
| | | EDL 933 | | | | | | |
| | | genome/ | | | | | Identical or Related proteins | |
| | Nucleic | pO157 | | | | | expressed in vivo in: | |
| Amino | Acid | assocaiated | | | | | Calves as | Humans as |
| Acid SEQ | SEQ | with the | Protein/ | | No. | Percent | identified by | identified by |
| ID NO: | ID NO: | protein¹ | Function² | Cell localization | hits | coverage | STM⁴ | IVIAT⁴ |
| | | Backbone | EcnB/entericidin B; | Extracellular | 1 | 40% | — | — |
| | | | bacteriolytic lipoprotein; | | | | | |
| | | | putative toxin of | | | | | |
| | | | osmotically regulated | | | | | |
| | | | toxin-antitoxin system | | | | | |
| | | | associated with | | | | | |
| | | | programmed cell death | | | | | |
| | | Backbone | OmpA/outer membrane | Outer | 42 | 43% | — | — |
| | | | protein 3a (II*; G; d); | membrane | | | | |
| | | | adhesin | | | | | |
| | | Backbone | Lpp (Murein-lipoprotein)/ | Outer | 7 | 62% | — | MepA, MltB, |
| | | | major outer membrane, | membrane | | | | Slt/murein |
| | | | murein sacculus, | | | | | sacculus |
| | | | peptidoglycan | | | | | synthesis |
| | | | lipoprotein precursor | | | | | |
| | | | (OmpT-like) | | | | | |
| | | Backbone | OmpC/outer membrane | Outer | 4 | 10% | — | — |
| | | | protein 1b (Ib; c); outer | membrane | | | | |
| | | | membrane constituents | | | | | |
| | | O-island # 43 | Z1178 (Iha)/adhesin; | Outer | 4 | 8% | Z1182/ | — |
| | | | exogenous ferric | membrane | | | 1726 bp 5′ | |
| | | | siderophore receptor R4 | | | | of Z1178 | |
| | | | | | | | (Iha) | |
| | | | | | | | adhesin | |
| 165 | 166 | O-island # 140 | ChuA/ | Outer | 1 | 2% | — | HemK/ |
| | | | heme/hemoglobin | membrane | | | | heme |
| | | | receptor (Heme | | | | | biosynthesis, |
| | | | utilization/transport | | | | | CcmH/heme |
| | | | protein) | | | | | lyase |
| 167 | 168 | O-island # 36 | Z0975/putative tail | Outer | 3 | 2% | Z0990/type | Z0975 |
| | | | component of prophage | membrane | | | III secreted | |
| | | | CP-933K | | | | protein | |
| | | | | | | | encoded | |
| | | | | | | | within | |
| | | | | | | | prophage | |
| | | | | | | | CP-933K | |
| 169 | 170 | O-island # 52 | Z1931/outer membrane | Outer | 7 | 15% | Z1930/ | — |
| | | | protein 3b (a), protease | membrane | | | putative | |
| | | | VII/enzyme; outer | | | | protease | |
| | | | membrane | | | | encoded | |
| | | | constituents encoded | | | | within | |
| | | | within prophage CP- | | | | prophage | |
| | | | 933X | | | | CP-933X | |
| | | Backbone | TolC/Outer membrane | Outer | 3 | 5% | — | — |
| | | | channel; specific | membrane | | | | |
| | | | tolerance to colicin E1; | | | | | |
| | | | segregation of daughter | | | | | |
| | | | chromosomes; cell | | | | | |
| | | | division | | | | | |
| | | Backbone | FhuE/outer membrane | Outer | 2 | 5% | — | FhuA/outer |
| | | | receptor for ferric iron | membrane | | | | membrane |
| | | | uptake | | | | | protein |
| | | | | | | | | receptor for |
| | | | | | | | | ferrichrome, |
| | | | | | | | | colicin M, |
| | | | | | | | | and phages |
| | | | | | | | | T1, T5, and |
| | | | | | | | | phi80 |
| | | pO157 | EspP/serine protease; | Outer | 1 | 1% | — | — |
| | | | putative exoprotein- | membrane | | | | |
| | | | precursor | | | | | |
| | | Backbone | OmpF/outer membrane | Outer | 2 | 6% | — | — |
| | | | protein 1a (Ia; b; F); outer | membrane | | | | |
| | | | membrane constituents | | | | | |
| | | Backbone | LamB/phage lambda | Outer | 1 | 4% | — | — |
| | | | receptor protein; maltose | membrane | | | | |
| | | | high-affinity receptor; IS, | | | | | |
| | | | phage, Tn; transport of | | | | | |
| | | | small molecules: | | | | | |
| | | | carbohydrates, organic | | | | | |
| | | | acids, alcohols | | | | | |
| 171 | 172 | Backbone | HflK/protease specific | Outer | 2 | 6% | — | HflX/GTP- |
| | | | for phage lambda cII | membrane | | | | binding |
| | | | repressor; enzyme; | | | | | subunit of |
| | | | macromolecule | | | | | protease |
| | | | degradation: degradation | | | | | specific for |
| | | | of proteins, peptides | | | | | phage |
| | | | | | | | | lambda cII |
| | | | | | | | | repressor |
| | | O-island # 148 | EspB/LEE encoded | Outer | 4 | 23% | EspD/LEE | — |
| | | | type III secreted protein; | membrane | | | encoded | |
| | | | formation of pore along | | | | type III | |
| | | | with EspD on host cell | | | | secreted | |
| | | | membrane | | | | protein; | |
| | | | | | | | formation of | |
| | | | | | | | pore along | |
| | | | | | | | with EspB | |
| | | | | | | | on host cell | |
| | | | | | | | membrane | |
| 173 | 174 | Backbone | HlpA/histone-like | Outer | 1 | 6% | — | — |
| | | | protein, located in outer | membrane | | | | |
| | | | membrane or nucleoid; | | | | | |
| | | | factor, Nucleoid-related | | | | | |
| | | | functions | | | | | |
| | | Backbone | OmpX/outer membrane | Outer | 1 | 7% | — | — |
| | | | protein X; membrane; | membrane | | | | |
| | | | Cell envelop: Outer | | | | | |
| | | | membrane constituents | | | | | |
| 175 | 176 | Backbone | YeaF/hypothetical | Outer | 1 | 5% | — | YeaA/ |
| | | | protein; unknown | membrane | | | | hypothetical |
| | | | function | | | | | protein; |
| | | | | | | | | unknown |
| | | | | | | | | function |
| 177 | 178 | Backbone | CirA/outer membrane | Outer | 1 | 2% | — | CirA |
| | | | receptor for iron- | membrane | | | | |
| | | | regulated colicin I | | | | | |
| | | | receptor; porin; requires | | | | | |
| | | | tonB gene product; Cell | | | | | |
| | | | envelop: Outer | | | | | |
| | | | membrane constituents | | | | | |
| 179 | 180 | O-island # 36 | Z0955/hypothetical | Outer | 3 | 8% | — | — |
| | | | protein encoded by | membrane | | | | |
| | | | prophage CP-933K; | | | | | |
| | | | unknown function | | | | | |
| | | Backbone | FepA/outer membrane | Outer | 1 | 2% | — | — |
| | | | receptor for ferric | membrane | | | | |
| | | | enterobactin | | | | | |
| | | | (enterochelin) and | | | | | |
| | | | colicins B and D; | | | | | |
| | | | Transport of small | | | | | |
| | | | molecules: Cations | | | | | |
| | | Backbone | MalE/maltose-binding | Periplasm | 2 | 8% | — | Mal F/ |
| | | | protein; substrate | | | | | maltose |
| | | | recognition for transport | | | | | permease, |
| | | | and chemotaxis; | | | | | MalS/alpha |
| | | | transport of small | | | | | amylase, |
| | | | molecules: | | | | | MaLY/ |
| | | | carbohydrates, organic | | | | | enzyme that |
| | | | acids, alcohols | | | | | may degrade |
| | | | | | | | | or block |
| | | | | | | | | biosynthesis |
| | | | | | | | | of |
| | | | | | | | | endogenous |
| | | | | | | | | mal inducer, |
| | | | | | | | | MalZ/ |
| | | | | | | | | maltodextrin |
| | | | | | | | | glucosidase |
| | | Backbone | TolB/protein involved | Periplasm | 1 | 4% | — | — |
| | | | in the tonb-independent | | | | | |
| | | | uptake of group A | | | | | |
| | | | colicins; colicin-related | | | | | |
| | | | functions | | | | | |
| | | Backbone | FkpA/FKBP-type | Periplasm | 1 | 6% | — | — |
| | | | peptidyl-prolyl cis-trans | | | | | |
| | | | isomerase | | | | | |
| | | | (rotamase); enzyme; | | | | | |
| | | | macromolecule | | | | | |
| | | | synthesis, modification: | | | | | |
| | | | proteins, translation, and | | | | | |
| | | | modification | | | | | |
| 181 | 182 | Backbone | YbeJ/putative | Periplasm | 1 | 6% | — | — |
| | | | periplasmic binding | | | | | |
| | | | transport protein; | | | | | |
| | | | putative transport | | | | | |
| | | Backbone | GlnH/periplasmic | Periplasm | 1 | 8% | — | GlnG/ |
| | | | glutamine-binding | | | | | response |
| | | | protein; permease; | | | | | regulator for |
| | | | transport; transport of | | | | | gln operon; |
| | | | small molecules: amino | | | | | interacts with |
| | | | acids, amines | | | | | sensor GlnL |
| | | | | | | | | GlnL/ |
| | | | | | | | | histidine |
| | | | | | | | | protein |
| | | | | | | | | kinase sensor |
| | | | | | | | | for the GlnG |
| | | | | | | | | regulator |
| | | | | | | | | Both are |
| | | | | | | | | involved in |
| | | | | | | | | glutamine |
| | | | | | | | | biosynthesis |
| 183 | 184 | Backbone | Mdh/malate | Periplasm | 1 | 4% | — | — |
| | | | dehydrogenase; enzyme; | | | | | |
| | | | energy metabolism, | | | | | |
| | | | carbon: TCA cycle | | | | | |
| 185 | 186 | Backbone | Z2267/hypothetical | Periplasm | 4 | 10% | — | — |
| | | | protein yncE precursor; | | | | | |
| | | | putative receptor | | | | | |
| 187 | 188 | Backbone | YaeC/putative | Periplasm | 1 | 6% | — | — |
| | | | lipoprotein, D- | | | | | |
| | | | methionine transport | | | | | |
| | | | protein; D-methionine- | | | | | |
| | | | binding lipoprotein metQ | | | | | |
| | | | precursor | | | | | |
| 189 | 190 | Backbone | SdhA/succinate | Periplasm | 1 | 3% | — | — |
| | | | dehydrogenase, | | | | | |
| | | | flavoprotein subunit; | | | | | |
| | | | enzyme; energy | | | | | |
| | | | metabolism, carbon: | | | | | |
| | | | TCA cycle | | | | | |
| 191 | 192 | Backbone | DppA/dipeptide | Periplasm | 1 | 2% | — | — |
| | | | transport protein; | | | | | |
| | | | transport; Transport of | | | | | |
| | | | large molecules: Protein, | | | | | |
| | | | peptide secretion | | | | | |
| 193 | 194 | Backbone | YahO/hypothetical | Periplasm | 1 | 9% | — | — |
| | | | protein; unknown | | | | | |
| | | | function | | | | | |
| 195 | 196 | O-island # 7 | Z0269/hypothetical | Periplasm | 3 | 4% | — | — |
| | | | protein; unknown | | | | | |
| | | | function (Rhs Element | | | | | |
| | | | Associated) | | | | | |
| | | Backbone | RbsB/D-ribose | Periplasm | 3 | 17% | — | — |
| | | | periplasmic binding | | | | | |
| | | | protein; Transport of | | | | | |
| | | | small molecules: | | | | | |
| | | | Carbohydrates, organic | | | | | |
| | | | acids, alcohols | | | | | |
| 197 | 198 | Backbone | OsmY/ | Periplasm | 7 | 26% | — | OsmY |
| | | | hyperosmotically | | | | | |
| | | | inducible periplasmic | | | | | |
| | | | protein; osmotic | | | | | |
| | | | adaptation | | | | | |
| 199 | 200 | Backbone | Z3065/hypothetical | Periplasm | 1 | 6% | — | — |
| | | | protein; unknown | | | | | |
| | | | function | | | | | |
| | | Backbone | Prc/carboxy-terminal | Periplasm | 1 | 2% | — | — |
| | | | protease for penicillin- | | | | | |
| | | | binding protein 3; | | | | | |
| | | | Macromolecule | | | | | |
| | | | degradation: Degradation | | | | | |
| | | | of proteins, peptides, | | | | | |
| | | | glyco | | | | | |
| 201 | 202 | Backbone | ArgT/lysine-, arginine-, | Periplasm | 1 | 6% | — | — |
| | | | ornithine-binding | | | | | |
| | | | periplasmic protein; | | | | | |
| | | | transport; Transport of | | | | | |
| | | | small molecules: Amino | | | | | |
| | | | acids, amines | | | | | |
| | | Backbone | OppA/oligopeptide | Periplasm | 5 | 8% | — | OppB/ |
| | | | transport; periplasmic | | | | | oligopeptide |
| | | | binding protein; | | | | | transport |
| | | | transport; Transport of | | | | | permease |
| | | | large molecules: Protein, | | | | | protein; |
| | | | peptide secretion | | | | | transport; |
| | | | | | | | | Transport of |
| | | | | | | | | large |
| | | | | | | | | molecules: |
| | | | | | | | | Protein, |
| | | | | | | | | peptide |
| | | | | | | | | secretion |
| | | Backbone | SodC/superoxide | Periplasm | 1 | 6% | — | — |
| | | | dismutase precursor (Cu—Zn); | | | | | |
| | | | enzyme; Protection | | | | | |
| | | | responses: Detoxification | | | | | |
| 203 | 204 | Backbone | XasA (GadC)/acid | Inner membrane | 6 | 3% | — | — |
| | | | sensitivity protein, | | | | | |
| | | | putative transporter; | | | | | |
| | | | Probable | | | | | |
| | | | glutamate/gamma- | | | | | |
| | | | aminobutyrate antiporter | | | | | |
| 205 | 206 | Backbone | PspA/phage shock | Inner membrane | 1 | 7% | — | — |
| | | | protein A; prophage and | | | | | |
| | | | phage-related functions; | | | | | |
| | | | adaptations, atypical | | | | | |
| | | | conditions; negative | | | | | |
| | | | regulatory gene for the | | | | | |
| | | | psp operon | | | | | |
| | | Backbone | AtpF/ATP synthase, F0 | Inner membrane | 1 | 8% | — | — |
| | | | sector, B chain; enzyme; | | | | | |
| | | | ATP-proton motive | | | | | |
| | | | force, interconversion | | | | | |
| | | Backbone | AtpB/membrane-bound | Inner membrane | 3 | 21% | — | — |
| | | | ATP synthase, F0 sector, | | | | | |
| | | | subunit A; enzyme; ATP- | | | | | |
| | | | proton motive force | | | | | |
| | | | interconversion | | | | | |
| 207 | 208 | Backbone | YleB/putative 2- | Inner membrane | 1 | 5% | — | YleB |
| | | | polyprenyl-6- | | | | | |
| | | | methoxyphenol | | | | | |
| | | | hydroxylase and related | | | | | |
| | | | FAD-dependent | | | | | |
| | | | oxidoreductase; | | | | | |
| | | | unknown function | | | | | |
| 209 | 210 | Backbone | CysK/cysteine synthase | Inner | 2 | 8% | — | — |
| | | | A, O-acetylserine | membrane | | | | |
| | | | sulfhydrolase A; enzyme; | | | | | |
| | | | Amino acid biosynthesis: | | | | | |
| | | | Cysteine | | | | | |
| 211 | 212 | Backbone | DnaC/chromosome | Inner | 1 | 5% | — | — |
| | | | replication; initiation and | membrane | | | | |
| | | | chain elongation; | | | | | |
| | | | putative enzyme; | | | | | |
| | | | Macromolecule | | | | | |
| | | | synthesis, modification: | | | | | |
| | | | DNA - replication, | | | | | |
| | | | repair, restr./modific'n | | | | | |
| 213 | 214 | Backbone | DsbB/reoxidizes DsbA | Inner | 1 | 8% | — | DsbA/ |
| | | | protein following | membrane | | | | protein |
| | | | formation of disulfide | | | | | disulfide |
| | | | bond in P-ring of | | | | | isomerase I, |
| | | | flagella; enzyme; Cell | | | | | essential for |
| | | | exterior constituents: | | | | | cytochrome c |
| | | | Surface | | | | | synthesis and |
| | | | structures | | | | | formate- |
| | | | | | | | | dependent |
| | | | | | | | | reduction; |
| | | | | | | | | Proteins - |
| | | | | | | | | translation |
| | | | | | | | | and |
| | | | | | | | | modification |
| 215 | 216 | Backbone | ElaB/hypothetical | Inner | 3 | 28% | — | — |
| | | | protein; unknown | membrane | | | | |
| | | | function | | | | | |
| 217 | 218 | Backbone | GatD/galactitol-1- | Inner | 1 | 4% | — | — |
| | | | phosphate | membrane | | | | |
| | | | dehydrogenase; enzyme; | | | | | |
| | | | Degradation of small | | | | | |
| | | | molecules: Carbon | | | | | |
| | | | compounds | | | | | |
| 219 | 220 | Backbone | KefC/K+ efflux | Inner membrane | 1 | 3% | — | KefC |
| | | | antiporter, glutathione- | | | | | |
| | | | regulated; transport; | | | | | |
| | | | Transport of small | | | | | |
| | | | molecules: Cations | | | | | |
| 221 | 222 | O-island # 57 | Z2112/putative ClpP- | Inner | 1 | 7% | — | — |
| | | | like protease encoded | membrane | | | | |
| | | | within prophage CP- | | | | | |
| | | | 933O; putative enzyme; | | | | | |
| | | | Macromolecule | | | | | |
| | | | degradation: Degradation | | | | | |
| | | | of proteins, peptides, | | | | | |
| | | | glyco (Phage or | | | | | |
| | | | Prophage Related) | | | | | |
| | | Backbone | LacI/transcriptional | Inner | 3 | 11% | — | — |
| | | | repressor of the lac | membrane | | | | |
| | | | operon; regulator; | | | | | |
| | | | Degradation of small | | | | | |
| | | | molecules: Carbon | | | | | |
| | | | compounds | | | | | |
| 223 | 224 | Backbone | GatZ/putative tagatose | Inner | 4 | 10% | — | — |
| | | | 6-phosphate kinase 1; | membrane | | | | |
| | | | unknown function | | | | | |
| 225 | 226 | Backbone | AcnB/aconitate hydrase | Inner | 1 | 2% | — | — |
| | | | B; Energy metabolism, | membrane | | | | |
| | | | carbon: TCA cycle | | | | | |
| 227 | 228 | Backbone | CydA/cytochrome d | Inner | 1 | 3% | — | — |
| | | | terminal oxidase, | membrane | | | | |
| | | | polypeptide subunit I; | | | | | |
| | | | Energy metabolism, | | | | | |
| | | | carbon: Electron | | | | | |
| | | | transport | | | | | |
| 229 | 230 | Backbone | CreD/tolerance to | Inner | 1 | 6% | — | — |
| | | | colicin E2; putative | membrane | | | | |
| | | | membrane; Colicin- | | | | | |
| | | | related functions | | | | | |
| 231 | 232 | Backbone | YgjD/putative O- | Inner | 1 | 5% | — | — |
| | | | sialoglycoprotein | membrane | | | | |
| | | | endopeptidase; unknown | | | | | |
| | | | function | | | | | |
| 233 | 234 | Backbone | Z5187/putative | Inner | 1 | 8% | — | — |
| | | | replicase; | membrane | | | | |
| | | | Macromolecule | | | | | |
| | | | synthesis, modification: | | | | | |
| | | | DNA - replication, | | | | | |
| | | | repair, restr./modific'n | | | | | |
| 235 | 236 | Backbone | YtfQ/putative LACI- | Inner | 1 | 4% | — | — |
| | | | type transcriptional | membrane | | | | |
| | | | regulator, unknown | | | | | |
| | | | function | | | | | |
| | | Backbone | FklB/FKBP-type 22 KD | Inner | 5 | 17% | — | — |
| | | | peptidyl-prolyl cis-trans | membrane | | | | |
| | | | isomerase (rotamase); | | | | | |
| | | | Macromolecule | | | | | |
| | | | synthesis, | | | | | |
| | | | modification:Proteins - | | | | | |
| | | | translation and | | | | | |
| | | | modification | | | | | |
| | | Backbone | RpsA/30S ribosomal | Inner | 2 | 6% | — | — |
| | | | subunit protein S1; | membrane | | | | |
| | | | structural component; | | | | | |
| | | | Macromolecule | | | | | |
| | | | synthesis, modification: | | | | | |
| | | | Ribosomal proteins | | | | | |
| | | Backbone | Ssb/ssDNA-binding | Inner | 5 | 20% | — | — |
| | | | protein; factor; | membrane | | | | |
| | | | Macromolecule | | | | | |
| | | | synthesis, modification: | | | | | |
| | | | DNA - replication, | | | | | |
| | | | repair, | | | | | |
| | | | restriction./modification | | | | | |
| 237 | 238 | Backbone | SucC/succinyl-CoA | Inner | 2 | 9% | — | — |
| | | | synthetase, beta subunit; | membrane | | | | |
| | | | enzyme; Energy | | | | | |
| | | | metabolism, carbon: | | | | | |
| | | | TCA cycle | | | | | |
| | | Backbone | RplK/50S ribosomal | Inner | 5 | 26% | — | — |
| | | | subunit protein L11; | membrane | | | | |
| | | | structural component; | | | | | |
| | | | Macromolecule | | | | | |
| | | | synthesis, modification: | | | | | |
| | | | Ribosomal proteins | | | | | |
| 239 | 240 | Backbone | TpiA/triosephosphate | Inner | 1 | 6% | — | — |
| | | | isomerase; enzyme; | membrane | | | | |
| | | | Energy metabolism, | | | | | |
| | | | carbon: Glycolysis | | | | | |
| 241 | 242 | Backbone | YgaM/hypothetical | Inner | 2 | 12% | — | — |
| | | | protein; unknown | membrane | | | | |
| | | | function | | | | | |
| | | Backbone | TrxA/thioredoxin 1; | Cytoplasm | 18 | 43% | TrxC/ | Ggt/ |
| | | | enzyme; biosynthesis of | | | | putative | biosynthesis |
| | | | cofactors, carriers: | | | | thioredoxin- | of cofactors, |
| | | | thioredoxin, | | | | like protein | carriers: |
| | | | glutaredoxin, glutathione | | | | | thioredoxin, |
| | | | | | | | | glutaredoxin, |
| | | | | | | | | glutathione |
| 243 | 244 | Backbone | DeaD/inducible ATP- | Cytoplasm | 13 | 5% | — | — |
| | | | independent RNA | | | | | |
| | | | helicase; enzyme; | | | | | |
| | | | macromolecule | | | | | |
| | | | synthesis, modification: | | | | | |
| | | | RNA synthesis, | | | | | |
| | | | modification, DNA | | | | | |
| | | | transcription | | | | | |
| | | Backbone | DpS/DNA protection | Cytoplasm | 2 | 22% | — | — |
| | | | during starvation protein; | | | | | |
| | | | global regulatory | | | | | |
| | | | functions | | | | | |
| | | Backbone | Tpx/thiol peroxidase; | Cytoplasm | 1 | 9% | — | — |
| | | | enzyme; protection | | | | | |
| | | | responses: detoxification | | | | | |
| 245 | 246 | Backbone | AceE/pyruvate | Cytoplasm | 2 | 4% | — | — |
| | | | dehydrogenase | | | | | |
| | | | (decarboxylase | | | | | |
| | | | component); enzyme; | | | | | |
| | | | energy metabolism | | | | | |
| 247 | 248 | Backbone | Gnd/gluconate-6- | Cytoplasm | 2 | 6% | — | — |
| | | | phosphate | | | | | |
| | | | dehydrogenase, | | | | | |
| | | | decarboxylating; | | | | | |
| | | | enzyme; energy | | | | | |
| | | | metabolism, carbon: | | | | | |
| | | | oxidative branch, pentose | | | | | |
| | | | pathway | | | | | |
| 249 | 250 | Backbone | MalP/maltodextrin | Cytoplasm | 3 | 5% | — | Mal F/ |
| | | | phosphorylase; enzyme; | | | | | maltose |
| | | | degradation of small | | | | | permease, |
| | | | molecules: carbon | | | | | MalS/alpha |
| | | | compounds | | | | | amylase, |
| | | | | | | | | MaLY/ |
| | | | | | | | | enzyme that |
| | | | | | | | | may degrade |
| | | | | | | | | or block |
| | | | | | | | | biosynthesis |
| | | | | | | | | of |
| | | | | | | | | endogenous |
| | | | | | | | | mal inducer, |
| | | | | | | | | MalZ/ |
| | | | | | | | | maltodextrin |
| | | | | | | | | glucosidase |
| | | Backbone | HflC/protease specific | Cytoplasm | 1 | 5% | — | HflX/GTP- |
| | | | for phage lambda cII | | | | | binding |
| | | | repressor; enzyme; | | | | | subunit of |
| | | | macromolecule | | | | | protease |
| | | | degradation: degradation | | | | | specific for |
| | | | of proteins, peptides | | | | | phage |
| | | | | | | | | lambda cII |
| | | | | | | | | repressor |
| | | Backbone | Eno/enolase; enzyme; | Cytoplasm | 2 | 7% | — | — |
| | | | energy metabolism, | | | | | |
| | | | carbon: glycolysis, | | | | | |
| | | | anaerobic respiration, | | | | | |
| | | | gluconeogenesis | | | | | |
| 251 | 252 | Backbone | MopB/GroES; 10 Kd | Cytoplasm | 1 | 13% | — | MopB |
| | | | chaperone binds to | | | | | |
| | | | Hsp60 in presence of | | | | | |
| | | | Mg-ATP, suppressing its | | | | | |
| | | | ATPase activity; folding | | | | | |
| | | | and ushering proteins: | | | | | |
| | | | Chaperones | | | | | |
| 253 | 254 | Backbone | Z3260/hypothetical | Cytoplasm | 1 | 6% | — | — |
| | | | protein; unknown | | | | | |
| | | | function; putative | | | | | |
| | | | fructose bis-phosphate | | | | | |
| | | | aldolase | | | | | |
| | | Backbone | Add/adenosine | Cytoplasm | 1 | 10% | — | — |
| | | | deaminase; enzyme; | | | | | |
| | | | central intermediary | | | | | |
| | | | metabolism: salvage of | | | | | |
| | | | nucleosides and | | | | | |
| | | | nucleotides | | | | | |
| | | Backbone | DnaK/chaperone | Cytoplasm | 4 | 9% | — | Z2654/ |
| | | | Hsp70; DNA | | | | | putative |
| | | | biosynthesis; | | | | | chaperone |
| | | | autoregulated heat shock | | | | | distantly |
| | | | proteins; folding and | | | | | related to |
| | | | ushering proteins: | | | | | Hsp70 |
| | | | chaperones | | | | | |
| 255 | 256 | Backbone | Pnp/polynucleotide | Cytoplasm | 2 | 6% | — | — |
| | | | phosphorylase; cytidylate | | | | | |
| | | | kinase; macromolecule | | | | | |
| | | | synthesis, modification: | | | | | |
| | | | RNA synthesis, | | | | | |
| | | | modification, DNA | | | | | |
| | | | transcription | | | | | |
| | | Backbone | RpsE/30S ribosomal | Cytoplasm | 2 | 13% | — | — |
| | | | subunit protein S5; | | | | | |
| | | | protein synthesis, | | | | | |
| | | | modification | | | | | |
| | | Backbone | RpsG/30S ribosomal | Cytoplasm | 1 | 8% | — | — |
| | | | subunit protein S7, | | | | | |
| | | | initiates assembly; | | | | | |
| | | | protein synthesis, | | | | | |
| | | | modification | | | | | |
| | | Backbone | RpsH/30S ribosomal | Cytoplasm | 1 | 12% | — | — |
| | | | subunit protein S8, | | | | | |
| | | | initiates assembly; | | | | | |
| | | | protein synthesis, | | | | | |
| | | | modification | | | | | |
| 257 | 258 | Backbone | TnaA/tryptophanase; | Cytoplasm | 3 | 9% | — | — |
| | | | degradation of small | | | | | |
| | | | molecules: amino acids | | | | | |
| | | Backbone | LpdA/lipoamide | Cytoplasm | 4 | 17% | — | — |
| | | | dehydrogenase (NADH); | | | | | |
| | | | component of 2- | | | | | |
| | | | oxodehydrogenase and | | | | | |
| | | | pyruvate complexes; L- | | | | | |
| | | | protein of glycine | | | | | |
| | | | cleavage complex; | | | | | |
| | | | enzyme; energy | | | | | |
| | | | metabolism, carbon: | | | | | |
| | | | pyruvate dehydrogenase | | | | | |
| | | Backbone | TufA/protein chain | Cytoplasm | 2 | 10% | — | — |
| | | | elongation factor EF-Tu; | | | | | |
| | | | macromolecule | | | | | |
| | | | synthesis, modification: | | | | | |
| | | | proteins - translation and | | | | | |
| | | | modification | | | | | |
| | | Backbone | Ftn (FtnA)/ferritin (an | Cytoplasm | 1 | 9% | — | — |
| | | | iron storage protein); | | | | | |
| | | | carrier; transport of small | | | | | |
| | | | molecules: cations | | | | | |
| | | Backbone | DapD/2,3,4,5- | Cytoplasm | 1 | 5% | — | DapD |
| | | | tetrahydropyridine-2- | | | | | |
| | | | carboxylate N- | | | | | |
| | | | succinyltransferase; | | | | | |
| | | | enzyme; amino acid | | | | | |
| | | | biosynthesis: lysine | | | | | |
| 259 | 260 | Backbone | AceF/pyruvate | Cytoplasm | 3 | 6% | — | — |
| | | | dehydrogenase (dihydro- | | | | | |
| | | | lipoyltransacetylase | | | | | |
| | | | component); enzyme; | | | | | |
| | | | energy metabolism, | | | | | |
| | | | carbon: pyruvate | | | | | |
| | | | dehydrogenase | | | | | |
| | | Backbone | AdhP/alcohol | Cytoplasm | 1 | 7% | — | AdhP |
| | | | dehydrogenase; enzyme; | | | | | |
| | | | energy metabolism, | | | | | |
| | | | carbon: anaerobic | | | | | |
| | | | respiration | | | | | |
| 261 | 262 | Backbone | Z1099/hypothetical | Cytoplasm | 1 | 13% | — | — |
| | | | protein; unknown | | | | | |
| | | | function | | | | | |
| | | Backbone | Tsf/protein chain | Cytoplasm | 1 | 7% | — | — |
| | | | elongation factor EF-Ts; | | | | | |
| | | | macromolecule | | | | | |
| | | | synthesis, modification: | | | | | |
| | | | proteins - translation and | | | | | |
| | | | modification | | | | | |
| | | Backbone | RpoE/RNA polymerase, | Cytoplasm | 1 | 9% | — | — |
| | | | sigma-E factor; heat | | | | | |
| | | | shock and oxidative | | | | | |
| | | | stress; regulator; global | | | | | |
| | | | regulatory functions | | | | | |
| 263 | 264 | Backbone | YqjD/hypothetical | Cytoplasm | 1 | 18% | — | — |
| | | | protein; unknown | | | | | |
| | | | function | | | | | |
| | | Backbone | FabG/3-oxoacyl-[acyl- | Cytoplasm | 1 | 10% | — | — |
| | | | carrier-protein] | | | | | |
| | | | reductases; enzyme; fatty | | | | | |
| | | | acid and phosphatidic | | | | | |
| | | | acid biosynthesis | | | | | |
| 265 | 266 | Backbone | CreA/hypothetical | Cytoplasm | 1 | 9% | — | — |
| | | | protein; unknown | | | | | |
| | | | function | | | | | |
| | | Backbone | LacZ/beta-D- | Cytoplasm | 1 | 6% | — | — |
| | | | galactosidase; enzyme; | | | | | |
| | | | degradation of small | | | | | |
| | | | molecules: carbon | | | | | |
| | | | compounds | | | | | |
| 267 | 268 | Backbone | OrdL/putative | Cytoplasm | 1 | 4% | Z2702/ | YhjL, Z2723, |
| | | | oxidoreductase; function | | | | putative | Z3401/ |
| | | | unknown | | | | oxido- | putative |
| | | | | | | | reductase, | oxido- |
| | | | | | | | Fe—S subunit | reductase |
| | | Backbone | FldA/flavodoxin 1; | Cytoplasm | 1 | 11% | — | Z4018/ |
| | | | enzyme; energy | | | | | putative |
| | | | metabolism, carbon: | | | | | flavodoxin |
| | | | electron transport | | | | | |
| | | Backbone | FusA/GTP-binding | Cytoplasm | 1 | 2% | — | — |
| | | | protein chain elongation | | | | | |
| | | | factor EF-G; | | | | | |
| | | | macromolecule | | | | | |
| | | | synthesis, modification: | | | | | |
| | | | proteins - translation and | | | | | |
| | | | modification | | | | | |
| | | Backbone | RpoA/RNA | Cytoplasm | 1 | 4% | — | — |
| | | | polymerase, alpha | | | | | |
| | | | subunit; enzyme; | | | | | |
| | | | macromolecule | | | | | |
| | | | synthesis, modification: | | | | | |
| | | | RNA synthesis, | | | | | |
| | | | modification, DNA | | | | | |
| | | | transcription | | | | | |
| 269 | 270 | Backbone | YhdH/putative | Cytoplasm | 1 | 4% | — | YgjL, YidS, |
| | | | dehydrogenase; unknown | | | | | YliL, Z3775, |
| | | | function | | | | | Z4220/ |
| | | | | | | | | putative |
| | | | | | | | | dehydrogenase |
| 271 | 272 | Backbone | SucA/2-oxoglutarate | Cytoplasm | 1 | 2% | — | — |
| | | | dehydrogenase | | | | | |
| | | | (decarboxylase | | | | | |
| | | | component); enzyme; | | | | | |
| | | | energy metabolism, | | | | | |
| | | | carbon: TCA cycle | | | | | |
| | | Backbone | AdhE/CoA-linked | Cytoplasm | 1 | 2% | — | — |
| | | | acetaldehyde | | | | | |
| | | | dehydrogenase and iron- | | | | | |
| | | | dependent alcohol | | | | | |
| | | | dehydrogenase; | | | | | |
| | | | pyruvate-formate-lyase | | | | | |
| | | | deactivase; enzyme; | | | | | |
| | | | energy metabolism, | | | | | |
| | | | carbon: fermentation | | | | | |
| | | Backbone | Pgk/phosphoglycerate | Cytoplasm | 1 | 6% | — | — |
| | | | kinase; enzyme; energy | | | | | |
| | | | metabolism, carbon: | | | | | |
| | | | glycolysis | | | | | |
| | | Backbone | RplO/50S ribosomal | Cytoplasm | 1 | 11% | — | — |
| | | | protein L15; protein | | | | | |
| | | | synthesis, modification | | | | | |
| | | Backbone | RplN/50S ribosomal | Cytoplasm | 2 | 33% | — | — |
| | | | protein L14; protein | | | | | |
| | | | synthesis, modification | | | | | |
| | | Backbone | RplL/50S ribosomal | Cytoplasm | 2 | 10% | — | — |
| | | | protein L7/L12; protein | | | | | |
| | | | synthesis, modification | | | | | |
| | | Backbone | RplJ/50S ribosomal | Cytoplasm | 1 | 9% | — | — |
| | | | protein L10; protein | | | | | |
| | | | synthesis, modification | | | | | |
| | | Backbone | RplF/50S ribosomal | Cytoplasm | 1 | 9% | — | — |
| | | | protein L6; protein | | | | | |
| | | | synthesis, modification | | | | | |
| | | Backbone | RplC/50S ribosomal | Cytoplasm | 1 | 10% | — | — |
| | | | protein L3; protein | | | | | |
| | | | synthesis, modification | | | | | |
| | | Backbone | YnaF/putative universal | Cytoplasm | 1 | 11% | — | — |
| | | | stress protein; unknown | | | | | |
| | | | function | | | | | |
| 273 | 274 | Backbone | MalQ/4-alpha- | Cytoplasm | 2 | 6% | — | Mal F/ |
| | | | glucanotransferase | | | | | maltose |
| | | | (amylomaltase); enzyme; | | | | | permease, |
| | | | macromolecule | | | | | MalS/alpha |
| | | | degradation: degradation | | | | | amylase, |
| | | | of polysaccharides | | | | | MaLY/ |
| | | | | | | | | enzyme that |
| | | | | | | | | may degrade |
| | | | | | | | | or block |
| | | | | | | | | biosynthesis |
| | | | | | | | | of |
| | | | | | | | | endogenous |
| | | | | | | | | mal inducer, |
| | | | | | | | | MalZ/ |
| | | | | | | | | maltodextrin |
| | | | | | | | | glucosidase |
| 275 | 276 | Backbone | YhbP/hypothetical | Cytoplasm | 1 | 8% | — | — |
| | | | protein; unknown | | | | | |
| | | | function | | | | | |
| 277 | 278 | Backbone | OsmC/osmotically | Cytoplasm | 1 | 15% | — | OsmY/ |
| | | | inducible protein; | | | | | hyperosmotically |
| | | | phenotype; osmotic | | | | | inducible |
| | | | adaptation | | | | | protein; |
| | | | | | | | | osmotic |
| | | | | | | | | adaptation |
| 279 | 280 | Backbone | Hns/DNA-binding | Cytoplasm | 3 | 17% | — | — |
| | | | protein HLP-II (HU, | | | | | |
| | | | BH2, HD, NS); | | | | | |
| | | | pleiotropic regulator; | | | | | |
| | | | regulator; basic proteins - | | | | | |
| | | | synthesis, | | | | | |
| | | | modification | | | | | |
| 281 | 282 | Backbone | SbmC/hypothetical | Cytoplasm | 1 | 12% | — | — |
| | | | protein; unknown | | | | | |
| | | | function | | | | | |
| | | Backbone | AtpA/membrane-bound | Cytoplasm | 11 | 23% | — | — |
| | | | ATP synthase, F1 sector, | | | | | |
| | | | alpha-subunit; enzyme; | | | | | |
| | | | ATP-proton motive force | | | | | |
| | | | interconversion | | | | | |
| | | Backbone | AtpG/membrane-bound | Cytoplasm | 1 | 4% | — | — |
| | | | ATP synthase, F1 sector, | | | | | |
| | | | gamma-subunit; | | | | | |
| | | | enzyme; ATP-proton | | | | | |
| | | | motive force | | | | | |
| | | | interconversion | | | | | |
| | | Backbone | MopA/GroEL, | Cytoplasm | 6 | 17% | — | — |
| | | | chaperone Hsp60, | | | | | |
| | | | peptide-dependent | | | | | |
| | | | ATPase, heat shock | | | | | |
| | | | protein; Folding and | | | | | |
| | | | ushering proteins: | | | | | |
| | | | Chaperones | | | | | |
| | | Backbone | Crp/cyclic AMP | Cytoplasm | 2 | 12% | — | — |
| | | | receptor protein; | | | | | |
| | | | regulator; Global | | | | | |
| | | | regulatory functions | | | | | |
| | | Backbone | CspA/cold shock | Cytoplasm | 1 | 20% | — | — |
| | | | protein 7.4, | | | | | |
| | | | transcriptional activator | | | | | |
| | | | of hns; regulator; | | | | | |
| | | | Adaptations, atypical | | | | | |
| | | | conditions | | | | | |
| | | Backbone | CspC/cold shock | Cytoplasm | 3 | 20% | 5′of cspC | — |
| | | | protein; unknown | | | | | |
| | | | function | | | | | |
| 283 | 284 | Backbone | CysN/ATP-sulfurylase | Cytoplasm | 1 | 3% | — | CysD/ |
| | | | (ATP:sulfate | | | | | ATP:sulfurylase |
| | | | adenylyltransferase), | | | | | (ATP:sulfate |
| | | | subunit 1, probably a | | | | | adenylyltransferase), |
| | | | GTPase; enzyme; Central | | | | | subunit 2; |
| | | | intermediary metabolism: | | | | | enzyme; |
| | | | Sulfur metabolism | | | | | Central |
| | | | | | | | | intermediary |
| | | | | | | | | metabolism: |
| | | | | | | | | Sulfur |
| | | | | | | | | metabolism |
| | | Backbone | HupA/DNA-binding | Cytoplasm | 4 | 32% | — | — |
| | | | protein HU-alpha (HU- | | | | | |
| | | | 2); Basic proteins - | | | | | |
| | | | synthesis, modification | | | | | |
| | | Backbone | FrdB/fumarate | Cytoplasm | 1 | 4% | — | — |
| | | | reductase, anaerobic, | | | | | |
| | | | iron-sulfur protein | | | | | |
| | | | subunit; enzyme; Energy | | | | | |
| | | | metabolism, carbon: | | | | | |
| | | | Anaerobic | | | | | |
| | | | respiration | | | | | |
| | | Backbone | GapA/glyceraldehyde- | Cytoplasm | 5 | 22% | — | — |
| | | | 3-phosphate | | | | | |
| | | | dehydrogenase A; | | | | | |
| | | | enzyme; Energy | | | | | |
| | | | metabolism, carbon: | | | | | |
| | | | Glycolysis | | | | | |
| 285 | 286 | Backbone | FolE/GTP | Cytoplasm | 2 | 13% | — | FolE |
| | | | cyclohydrolase I; | | | | | |
| | | | enzyme; Biosynthesis of | | | | | |
| | | | cofactors, carriers: Folic | | | | | |
| | | | acid | | | | | |
| | | Backbone | GlnA/glutamine | Cytoplasm | 1 | 3% | — | GlnG/ |
| | | | synthetase; enzyme; | | | | | response |
| | | | Amino acid biosynthesis: | | | | | regulator for |
| | | | Glutamine | | | | | gln operon; |
| | | | | | | | | interacts with |
| | | | | | | | | sensor GlnL |
| | | | | | | | | GlnL/ |
| | | | | | | | | histidine |
| | | | | | | | | protein |
| | | | | | | | | kinase sensor |
| | | | | | | | | for the GlnG |
| | | | | | | | | regulator |
| | | | | | | | | Both are |
| | | | | | | | | involved in |
| | | | | | | | | glutamine |
| | | | | | | | | biosynthesis |
| 287 | 288 | Backbone | GpmA/ | Cytoplasm | 2 | 11% | — | — |
| | | | phosphoglyceromutase 1; | | | | | |
| | | | enzyme; Energy | | | | | |
| | | | metabolism, carbon: | | | | | |
| | | | Glycolysis | | | | | |
| | | Backbone | GrpE/phage lambda | Cytoplasm | 1 | 8% | — | — |
| | | | replication; host DNA | | | | | |
| | | | synthesis; | | | | | |
| | | | heat shock protein; | | | | | |
| | | | protein repair; IS, phage, | | | | | |
| | | | Tn; Other or unknown | | | | | |
| | | Backbone | HisB/ | Cytoplasm | 1 | 5% | — | HisS/ |
| | | | imidazoleglycerolphosphate | | | | | histidine |
| | | | dehydratase and | | | | | tRNA |
| | | | histidinol-phosphate | | | | | synthetase; |
| | | | phosphatase; enzyme; | | | | | enzyme; |
| | | | Amino acid biosynthesis: | | | | | Amino acyl |
| | | | Histidine | | | | | tRNA syn; |
| | | | | | | | | tRNA |
| | | | | | | | | modification |
| | | Backbone | HslV/heat shock protein | Cytoplasm | 1 | 7% | — | — |
| | | | hslVU, proteasome- | | | | | |
| | | | related peptidase subunit; | | | | | |
| | | | enzyme; macromolecule | | | | | |
| | | | degradation: Degradation | | | | | |
| | | | of proteins, peptides, | | | | | |
| | | | glyco | | | | | |
| | | Backbone | IbpA/heat shock | Cytoplasm | 1 | 9% | — | — |
| | | | protein; factor; | | | | | |
| | | | Adaptations, atypical | | | | | |
| | | | conditions | | | | | |
| | | Backbone | IbpB/heat shock | Cytoplasm | 2 | 8% | — | — |
| | | | protein; factor; | | | | | |
| | | | Adaptations, atypical | | | | | |
| | | | conditions | | | | | |
| 289 | 290 | Backbone | InfC/Initiation factor | Cytoplasm | 2 | 34% | — | — |
| | | | IF-3; Macromolecule | | | | | |
| | | | synthesis, modification: | | | | | |
| | | | Proteins - translation and | | | | | |
| | | | modification | | | | | |
| 291 | 292 | Backbone | HimD/integration host | Cytoplasm | 2 | 13% | — | — |
| | | | factor (IHF), beta | | | | | |
| | | | subunit; site-specific | | | | | |
| | | | recombination; factor; | | | | | |
| | | | Macromolecule | | | | | |
| | | | synthesis, modification: | | | | | |
| | | | DNA - replication, | | | | | |
| | | | repair, restr./modific'n | | | | | |
| | | Backbone | AdK/adenylate kinase | Cytoplasm | 4 | 12% | — | — |
| | | | activity; pleiotropic | | | | | |
| | | | effects on glycerol-3- | | | | | |
| | | | phosphate | | | | | |
| | | | acyltransferase activity; | | | | | |
| | | | enzyme; Nucleotide | | | | | |
| | | | biosynthesis: Purine | | | | | |
| | | | ribonucleotide | | | | | |
| | | | biosynthesis | | | | | |
| | | Backbone | MalK/ATP-binding | Cytoplasm | 1 | 3% | — | Mal F/ |
| | | | component of transport | | | | | maltose |
| | | | system for maltose; | | | | | permease, |
| | | | transport; Transport of | | | | | MalS/alpha |
| | | | small molecules: | | | | | amylase, |
| | | | Carbohydrates, organic | | | | | MaLY/ |
| | | | acids, alcohols | | | | | enzyme that |
| | | | | | | | | may degrade |
| | | | | | | | | or block |
| | | | | | | | | biosynthesis |
| | | | | | | | | of |
| | | | | | | | | endogenous |
| | | | | | | | | mal inducer, |
| | | | | | | | | MalZ/ |
| | | | | | | | | maltodextrin |
| | | | | | | | | glucosidase |
| 293 | 294 | Backbone | MukE/hypothetical | Cytoplasm | 1 | 9% | — | — |
| | | | protein; Unknown | | | | | |
| | | | function | | | | | |
| | | Backbone | NuoB/NADH | Cytoplasm | 1 | 4% | — | NuoL/ |
| | | | dehydrogenase I chain B; | | | | | NADH de- |
| | | | enzyme; Energy | | | | | hydrogenase |
| | | | metabolism, carbon: | | | | | I chain L; |
| | | | Aerobic respiration | | | | | enzyme; |
| | | | | | | | | Energy |
| | | | | | | | | metabolism, |
| | | | | | | | | carbon: |
| | | | | | | | | Aerobic |
| | | | | | | | | respiration |
| | | | | | | | | NuoM/ |
| | | | | | | | | NADH de- |
| | | | | | | | | hydrogenase |
| | | | | | | | | I chain M; |
| | | | | | | | | enzyme; |
| | | | | | | | | Energy |
| | | | | | | | | metabolism, |
| | | | | | | | | carbon: |
| | | | | | | | | Aerobic |
| | | | | | | | | respiration |
| | | Backbone | NuoG/NADH | Cytoplasm | 1 | 1% | — | NuoL/ |
| | | | dehydrogenase I chain G; | | | | | NADH de- |
| | | | enzyme; Energy | | | | | hydrogenase |
| | | | metabolism, carbon: | | | | | I chain L; |
| | | | Aerobic respiration | | | | | enzyme; |
| | | | | | | | | Energy |
| | | | | | | | | metabolism, |
| | | | | | | | | carbon: |
| | | | | | | | | Aerobic |
| | | | | | | | | respiration |
| | | | | | | | | NuoM/ |
| | | | | | | | | NADH de- |
| | | | | | | | | hydrogenase |
| | | | | | | | | I chain M; |
| | | | | | | | | enzyme; |
| | | | | | | | | Energy |
| | | | | | | | | metabolism, |
| | | | | | | | | carbon: |
| | | | | | | | | Aerobic |
| | | | | | | | | respiration |
| | | Backbone | SucB/2-oxoglutarate | Cytoplasm | 7 | 12% | — | — |
| | | | dehydrogenase (dihydro- | | | | | |
| | | | lipoyltranssuccinase E2 | | | | | |
| | | | component); enzyme; | | | | | |
| | | | Energy metabolism, | | | | | |
| | | | carbon: TCA cycle | | | | | |
| 295 | 296 | Backbone | PckA/ | Cytoplasm | 1 | 1% | — | — |
| | | | phosphoenolpyruvate | | | | | |
| | | | carboxykinase; enzyme; | | | | | |
| | | | Central intermediary | | | | | |
| | | | metabolism: | | | | | |
| | | | Gluconeogenesis | | | | | |
| | | Backbone | PPk/polyphosphate | Cytoplasm | 1 | 3% | — | — |
| | | | kinase; enzyme; Central | | | | | |
| | | | intermediary metabolism: | | | | | |
| | | | Phosphorus compounds | | | | | |
| 297 | 298 | Backbone | ProQ/ProQ protein that | Cytoplasm | 1 | 30% | — | — |
| | | | influences osmotic | | | | | |
| | | | activation of ProP; | | | | | |
| | | | putative factor; Transport | | | | | |
| | | | of small molecules: | | | | | |
| | | | Amino acids, amines | | | | | |
| | | Backbone | PyrG/CTP synthetase; | Cytoplasm | 1 | 2% | — | — |
| | | | enzyme; Central | | | | | |
| | | | intermediary metabolism: | | | | | |
| | | | Nucleotide | | | | | |
| | | | interconversions | | | | | |
| | | O-island # 57 | Z2118/putative | Cytoplasm | 1 | 14% | — | — |
| | | | endopeptidase Rz of | | | | | |
| | | | prophage CP-933O; | | | | | |
| | | | putative enzyme; Lysis | | | | | |
| | | | (Phage or Prophage | | | | | |
| | | | Related) | | | | | |
| 299 | 300 | O-island # 50 | Z1824/hypothetical | Cytoplasm | 1 | 1% | — | — |
| | | | protein encoded by | | | | | |
| | | | prophage CP-933N; | | | | | |
| | | | unknown function | | | | | |
| | | Backbone | FumB/fumarase B = | Cytoplasm | 1 | 2% | — | — |
| | | | fumarate hydratase Class | | | | | |
| | | | I; anaerobic isozyme; | | | | | |
| | | | enzyme; Energy | | | | | |
| | | | metabolism, carbon: | | | | | |
| | | | TCA cycle | | | | | |
| | | Backbone | AceA/isocitrate lyase; | Cytoplasm | 1 | 4% | — | — |
| | | | enzyme; Central | | | | | |
| | | | intermediary metabolism: | | | | | |
| | | | Glyoxylate bypass | | | | | |
| | | Backbone | TopA/DNA | Cytoplasm | 1 | 1% | — | TopA |
| | | | topoisomerase type I, | | | | | |
| | | | omega protein; | | | | | |
| | | | Macromolecule | | | | | |
| | | | synthesis, modification: | | | | | |
| | | | DNA - replication, | | | | | |
| | | | repair, restr./modific'n | | | | | |
| | | Backbone | PepD/aminoacyl- | Cytoplasm | 2 | 5% | — | — |
| | | | histidine dipeptidase | | | | | |
| | | | (peptidase D); | | | | | |
| | | | Macromolecule | | | | | |
| | | | degradation: Degradation | | | | | |
| | | | of proteins, peptides, | | | | | |
| | | | glyco | | | | | |
| | | Backbone | Udp/uridine | Cytoplasm | 1 | 5% | — | — |
| | | | phosphorylase; Central | | | | | |
| | | | intermediary metabolism: | | | | | |
| | | | Salvage of nucleosides | | | | | |
| | | | and nucleotides | | | | | |
| 301 | 302 | Backbone | YgaU/hypothetical | Cytoplasm | 6 | 39% | — | — |
| | | | protein; unknown | | | | | |
| | | | function | | | | | |
| | | Backbone | DnaJ/chaperone with | Cytoplasm | 2 | 4% | — | — |
| | | | DnaK; heat shock | | | | | |
| | | | protein; Folding and | | | | | |
| | | | ushering proteins: | | | | | |
| | | | Chaperones | | | | | |
| 303 | 304 | Backbone | Crr/PTS system, | Cytoplasm | 4 | 62% | — | — |
| | | | glucose-specific IIA | | | | | |
| | | | component; Transport of | | | | | |
| | | | small molecules: | | | | | |
| | | | Carbohydrates, organic | | | | | |
| | | | acids, alcohols | | | | | |
| 305 | 306 | O-island # 122 | Z4317/unknown protein | Cytoplasm | 1 | 1% | — | — |
| | | | encoded by ISEc8; | | | | | |
| | | | Unknown function | | | | | |
| | | | (Insertion Sequence | | | | | |
| | | | Associated) | | | | | |
| 307 | 308 | Backbone | Z1419/hypothetical | Cytoplasm | 1 | 6% | — | — |
| | | | protein; unknown | | | | | |
| | | | function | | | | | |
| 309 | 310 | Backbone | yjgF/hypothetical | Cytoplasm | 1 | 8% | — | — |
| | | | protein; unknown | | | | | |
| | | | function | | | | | |
| 311 | 312 | Backbone | PpiB/peptidyl-prolyl | Cytoplasm | 1 | 8% | — | — |
| | | | cis-trans isomerase B | | | | | |
| | | | (rotamase B); | | | | | |
| | | | Macromolecule | | | | | |
| | | | synthesis, modification: | | | | | |
| | | | Proteins - translation and | | | | | |
| | | | modification | | | | | |
| 313 | 314 | Backbone | YlbA/hypothetical | Cytoplasm | 1 | 4% | — | — |
| | | | proteins; unknown | | | | | |
| | | | function | | | | | |
| 315 | 316 | Backbone | ChaC/cation transport | Cytoplasm | 1 | 8% | — | — |
| | | | regulator; Transport of | | | | | |
| | | | small molecules: Cations | | | | | |
| 317 | 318 | Backbone | SgaU/putative | Cytoplasm | 1 | 6% | — | SgaB/ |
| | | | hexulose-6-phosphate | | | | | hypothetical |
| | | | isomerase; putative | | | | | protein; |
| | | | enzyme; Central | | | | | unknown |
| | | | intermediary | | | | | function |
| | | | metabolism: Pool, | | | | | |
| | | | multipurpose | | | | | |
| | | | conversions of | | | | | |
| | | | intermediary metabolites | | | | | |
| 319 | 320 | Backbone | MelA/alpha- | Cytoplasm | 1 | 2% | — | — |
| | | | galactosidase; enzyme; | | | | | |
| | | | Degradation of small | | | | | |
| | | | molecules: Carbon | | | | | |
| | | | compounds | | | | | |
| | | Backbone | Z2778/putative | Cytoplasm | 1 | 3% | — | — |
| | | | aldehyde dehydrogenase; | | | | | |
| | | | unknown function | | | | | |
| | | Backbone | KatE/catalase; | Cytoplasm | 1 | 2% | — | — |
| | | | hydroperoxidase | | | | | |
| | | | HPII(III); enzyme; | | | | | |
| | | | Protection responses: | | | | | |
| | | | detoxification | | | | | |
| | | Backbone | RplY/50S ribosomal | Cytoplasm | 1 | 16% | — | — |
| | | | subunit protein L25; | | | | | |
| | | | structural component; | | | | | |
| | | | Macromolecule | | | | | |
| | | | synthesis, modification: | | | | | |
| | | | Ribosomal proteins | | | | | |
| 321 | 322 | Backbone | RhlB/putative ATP- | Cytoplasm | 1 | 2% | — | — |
| | | | dependent RNA helicase; | | | | | |
| | | | unknown function | | | | | |
| | | Backbone | Z0516/riboflavin | Cytoplasm | 1 | 10% | — | — |
| | | | synthase subunit beta; | | | | | |
| | | | 6,7-dimethyl-8- | | | | | |
| | | | ribityllumazine synthase/ | | | | | |
| | | | note = DMRL₋ synthase; | | | | | |
| | | | enzyme; Biosynthesis of | | | | | |
| | | | cofactors, carriers: | | | | | |
| | | | Riboflavin | | | | | |
| | | Backbone | RplA/50S ribosomal | Cytoplasm | 10 | 52% | — | — |
| | | | subunit protein L1, | | | | | |
| | | | regulates synthesis of L1 | | | | | |
| | | | and L11; structural | | | | | |
| | | | component; | | | | | |
| | | | Macromolecule | | | | | |
| | | | synthesis, modification: | | | | | |
| | | | Ribosomal proteins | | | | | |
| | | Backbone | RplP/50S ribosomal | Cytoplasm | 1 | 12% | — | — |
| | | | subunit protein L16; | | | | | |
| | | | structural component; | | | | | |
| | | | Macromolecule | | | | | |
| | | | synthesis, modification: | | | | | |
| | | | Ribosomal proteins | | | | | |
| | | Backbone | RplQ/50S ribosomal | Cytoplasm | 2 | 24% | — | — |
| | | | subunit protein L17; | | | | | |
| | | | structural component; | | | | | |
| | | | Macromolecule | | | | | |
| | | | synthesis, modification: | | | | | |
| | | | Ribosomal proteins | | | | | |
| | | Backbone | RplR/50S ribosomal | Cytoplasm | 2 | 9% | — | — |
| | | | subunit protein L18; | | | | | |
| | | | structural component; | | | | | |
| | | | Macromolecule | | | | | |
| | | | synthesis, modification: | | | | | |
| | | | Ribosomal proteins | | | | | |
| | | Backbone | RplS/50S ribosomal | Cytoplasm | 2 | 23% | — | — |
| | | | subunit protein L19; | | | | | |
| | | | structural component; | | | | | |
| | | | Macromolecule | | | | | |
| | | | synthesis, modification: | | | | | |
| | | | Ribosomal proteins | | | | | |
| | | Backbone | RplB/50S ribosomal | Cytoplasm | 1 | 5% | — | — |
| | | | subunit protein L2; | | | | | |
| | | | structural component; | | | | | |
| | | | Macromolecule | | | | | |
| | | | synthesis, | | | | | |
| | | | modification: Ribosomal | | | | | |
| | | | proteins | | | | | |
| | | Backbone | RplU/50S ribosomal | Cytoplasm | 1 | 14% | — | — |
| | | | subunit protein L21; | | | | | |
| | | | structural component; | | | | | |
| | | | Macromolecule | | | | | |
| | | | synthesis, modification: | | | | | |
| | | | Ribosomal proteins | | | | | |
| | | Backbone | RplX/50S ribosomal | Cytoplasm | 6 | 32% | — | — |
| | | | subunit protein L24; | | | | | |
| | | | structural component; | | | | | |
| | | | Macromolecule | | | | | |
| | | | synthesis, modification: | | | | | |
| | | | Ribosomal proteins | | | | | |
| | | Backbone | RpmA/50S ribosomal | Cytoplasm | 1 | 11% | — | — |
| | | | subunit protein L27; | | | | | |
| | | | structural component; | | | | | |
| | | | Macromolecule | | | | | |
| | | | synthesis, modification: | | | | | |
| | | | Ribosomal proteins | | | | | |
| | | Backbone | RpmB/50S ribosomal | Cytoplasm | 1 | 15% | — | — |
| | | | subunit protein L28; | | | | | |
| | | | structural component; | | | | | |
| | | | Macromolecule | | | | | |
| | | | synthesis, modification: | | | | | |
| | | | Ribosomal proteins | | | | | |
| | | Backbone | RplD/50S ribosomal | Cytoplasm | 4 | 21% | — | — |
| | | | subunit protein L4, | | | | | |
| | | | regulates expression of | | | | | |
| | | | S10 operon; structural | | | | | |
| | | | component; | | | | | |
| | | | Macromolecule | | | | | |
| | | | synthesis, modification: | | | | | |
| | | | Ribosomal proteins | | | | | |
| | | Backbone | RplE/50S ribosomal | Cytoplasm | 2 | 9% | — | — |
| | | | subunit protein L5; | | | | | |
| | | | structural component; | | | | | |
| | | | Macromolecule | | | | | |
| | | | synthesis, | | | | | |
| | | | modification: Ribosomal | | | | | |
| | | | proteins | | | | | |
| | | Backbone | RplL/50S ribosomal | Cytoplasm | 8 | 38% | — | — |
| | | | subunit protein L7/L12; | | | | | |
| | | | structural component; | | | | | |
| | | | Macromolecule | | | | | |
| | | | synthesis, modification: | | | | | |
| | | | Ribosomal proteins | | | | | |
| | | Backbone | RpiA/ribosephosphate | Cytoplasm | 2 | 17% | — | — |
| | | | isomerase, constitutive; | | | | | |
| | | | enzyme; Central | | | | | |
| | | | intermediary metabolism: | | | | | |
| | | | Non-oxidative branch, | | | | | |
| | | | pentose pathway | | | | | |
| | | Backbone | Frr/ribosome releasing | Cytoplasm | 1 | 11% | — | — |
| | | | factor; factor; | | | | | |
| | | | Macromolecule | | | | | |
| | | | synthesis, modification: | | | | | |
| | | | Proteins - translation and | | | | | |
| | | | modification | | | | | |
| | | Backbone | RpsJ/30S ribosomal | Cytoplasm | 3 | 40% | — | — |
| | | | subunit protein S10; | | | | | |
| | | | structural component; | | | | | |
| | | | Macromolecule | | | | | |
| | | | synthesis, modification: | | | | | |
| | | | Ribosomal proteins | | | | | |
| | | Backbone | RpsK/30S ribosomal | Cytoplasm | 2 | 18% | — | — |
| | | | subunit protein S11; | | | | | |
| | | | structural component; | | | | | |
| | | | Macromolecule | | | | | |
| | | | synthesis, modification: | | | | | |
| | | | Ribosomal proteins | | | | | |
| | | Backbone | RpsN/30S ribosomal | Cytoplasm | 1 | 12% | — | — |
| | | | subunit protein S14; | | | | | |
| | | | structural component; | | | | | |
| | | | Macromolecule | | | | | |
| | | | synthesis, modification: | | | | | |
| | | | Ribosomal proteins | | | | | |
| | | Backbone | RpsP/30S ribosomal | Cytoplasm | 6 | 41% | — | — |
| | | | subunit protein S16; | | | | | |
| | | | structural component; | | | | | |
| | | | Macromolecule | | | | | |
| | | | synthesis, modification: | | | | | |
| | | | Ribosomal proteins | | | | | |
| | | Backbone | RpsQ/30S ribosomal | Cytoplasm | 1 | 8% | — | — |
| | | | subunit protein S17; | | | | | |
| | | | structural component; | | | | | |
| | | | Macromolecule | | | | | |
| | | | synthesis, modification: | | | | | |
| | | | Ribosomal proteins | | | | | |
| | | Backbone | RpsS/30S ribosomal | Cytoplasm | 2 | 10% | — | — |
| | | | subunit protein S19; | | | | | |
| | | | structural component; | | | | | |
| | | | Macromolecule | | | | | |
| | | | synthesis, modification: | | | | | |
| | | | Ribosomal proteins | | | | | |
| | | Backbone | RpsB/30S ribosomal | Cytoplasm | 1 | 5% | — | — |
| | | | subunit protein S2; | | | | | |
| | | | structural component; | | | | | |
| | | | Macromolecule | | | | | |
| | | | synthesis, modification: | | | | | |
| | | | Ribosomal proteins | | | | | |
| | | Backbone | RpsU/30S ribosomal | Cytoplasm | 1 | 14% | — | — |
| | | | subunit protein S21; | | | | | |
| | | | structural component; | | | | | |
| | | | Macromolecule | | | | | |
| | | | synthesis, modification: | | | | | |
| | | | Ribosomal proteins | | | | | |
| | | Backbone | RpsC/30S ribosomal | Cytoplasm | 7 | 45% | — | — |
| | | | subunit protein S3; | | | | | |
| | | | structural component; | | | | | |
| | | | Macromolecule | | | | | |
| | | | synthesis, modification: | | | | | |
| | | | Ribosomal proteins | | | | | |
| | | Backbone | RpsD/30S ribosomal | Cytoplasm | 6 | 24% | — | — |
| | | | subunit protein S4; | | | | | |
| | | | structural component; | | | | | |
| | | | Macromolecule | | | | | |
| | | | synthesis, modification: | | | | | |
| | | | Ribosomal proteins | | | | | |
| | | Backbone | RpsL/30S ribosomal | Cytoplasm | 3 | 17% | — | — |
| | | | subunit protein S12; | | | | | |
| | | | structural component; | | | | | |
| | | | Macromolecule | | | | | |
| | | | synthesis, modification: | | | | | |
| | | | Ribosomal proteins | | | | | |
| 323 | 324 | Backbone | SodB/superoxide | Cytoplasm | 1 | 13% | — | — |
| | | | dismutase, iron; enzyme; | | | | | |
| | | | Protection responses: | | | | | |
| | | | Detoxification | | | | | |
| 325 | 326 | Backbone | SucD/succinyl-CoA | Cytoplasm | 2 | 8% | — | — |
| | | | synthetase, alpha subunit; | | | | | |
| | | | enzyme; Energy | | | | | |
| | | | metabolism, carbon: | | | | | |
| | | | TCA cycle | | | | | |
| 327 | 328 | Backbone | TalB/transaldolase B; | Cytoplasm | 6 | 19% | — | — |
| | | | enzyme; Central | | | | | |
| | | | intermediary metabolism: | | | | | |
| | | | Non-oxidative branch, | | | | | |
| | | | pentose pathway | | | | | |
| | | Backbone | Tig/trigger factor; a | Cytoplasm | 5 | 13% | — | — |
| | | | molecular chaperone | | | | | |
| | | | involved in cell division; | | | | | |
| | | | factor; Cell division | | | | | |
| 329 | 330 | Backbone | Z0751/hypothetical | Cytoplasm | 4 | 32% | — | — |
| | | | protein/unknown | | | | | |
| | | | function | | | | | |
| 331 | 332 | Backbone | WrbA/trp repressor | Cytoplasm | 1 | 10% | — | — |
| | | | binding protein; affects | | | | | |
| | | | association of trp | | | | | |
| | | | repressor and operator; | | | | | |
| | | | regulator; Amino acid | | | | | |
| | | | biosynthesis: Tryptophan | | | | | |
| 333 | 334 | Backbone | YaiE/hypothetical | Cytoplasm | 2 | 14% | — | — |
| | | | protein/unknown | | | | | |
| | | | function | | | | | |
| 335 | 336 | Backbone | YbaA/hypothetical | Cytoplasm | 1 | 15% | — | — |
| | | | protein/unknown | | | | | |
| | | | function | | | | | |
| 337 | 338 | Backbone | YdfH/hypothetical | Cytoplasm | 1 | 7% | — | — |
| | | | protein/unknown | | | | | |
| | | | function | | | | | |
| 339 | 340 | Backbone | YjbJ/hypothetical | Cytoplasm | 4 | 46% | — | — |
| | | | protein/unknown | | | | | |
| | | | function | | | | | |

¹Based on homology to the sequenced, E. coli O157 strain EDL933 genome, and plasmid, pO157.
²Putative functions assigned to hypothetical proteins using the Conserved Domain Database (CDD).
³Bacterial cell localization of proteins determined by PSORTb v.2.0/PSLpred/PSORTdb, and SignalP 3.0 prediction program.
⁴STM, signature tagged mutagenesis¹⁷; IVIAT, in vivo induced antigen technology¹¹.

All publications and patents mentioned in this specification are herein incorporated by reference to the same extent as if each individual publication or patent was specifically and individually indicated to be incorporated by reference.

What is claimed is:

1. A composition for stimulating an immune response against E. coli O157, said composition comprising substantially pure OsmY (SEQ ID NO: 109) or an immunogenic fragment thereof.

2. The composition of claim 1, said composition further comprising a pharmaceutically acceptable carrier or an adjuvant.

3. A method of inducing an immune response to E. Coli O157 in a mammal, the method comprising administering to the mammal the composition of claim 1.

4. A method of treating a mammal for an E. Coli O157 associated disease, said method comprising administering the composition of claim 1.

5. The method of claim 4, wherein said treatment is a prophylactic.

6. The method of claim 4, wherein said treatment is therapeutic.

7. The method of claim 3 or 4, wherein said mammal is a cattle or human.

8. A method for producing an antibody specific to OsmY (SEQ ID NO: 109); said method comprising the steps of
a) immunizing a mammal with said peptide or an immunogenic fragment thereof, and
b) purifying said antibody from the tissue of said mammal, or from a hybridoma made using said tissue.

9. The method of claim 8 wherein said antibody is a polyclonal antibody or fragment thereof.

10. The method of claim 8 wherein said antibody is a monoclonal antibody or fragment thereof.

11. A composition for stimulating an immune response against E. coli O157, said composition comprising substantially pure YleB (SEQ ID NO: 43) or an immunogenic fragment thereof.

12. The composition of claim 11, said composition further comprising a pharmaceutically acceptable carrier or an adjuvant.

13. A method of inducing an immune response to E. Coli O157 in a mammal, the method comprising administering to the mammal the composition of claim 11.

14. A method of treating a mammal for an E. Coli O157 associated disease, said method comprising administering the composition of claim 11.

15. The method of claim 14, wherein said treatment is a prophylactic.

16. The method of claim 14, wherein said treatment is therapeutic.

17. The method of claim 13 or 14, wherein said mammal is a cattle or human.

18. A method for producing an antibody specific to YleB (SEQ ID NO: 43); said method comprising the steps of
a) immunizing a mammal with said peptide or an immunogenic fragment thereof, and
b) purifying said antibody from the tissue of said mammal, or from a hybridoma made using said tissue.

19. The method of claim 18 wherein said antibody is a polyclonal antibody or fragment thereof.

20. The method of claim 18 wherein said antibody is a monoclonal antibody or fragment thereof.

21. A composition for stimulating an immune response against E. coli O157, said composition comprising substantially pure KefC (SEQ ID NO: 91) or an immunogenic fragment thereof.

22. The composition of claim 21, said composition further comprising a pharmaceutically acceptable carrier or an adjuvant.

23. A method of inducing an immune response to E. Coli O157 in a mammal, the method comprising administering to the mammal the composition of claim 21.

24. A method of treating a mammal for an E. Coli O157 associated disease, said method comprising administering the composition of claim 21.

25. The method of claim 24, wherein said treatment is a prophylactic.

26. The method of claim 24, wherein said treatment is therapeutic.

27. The method of claim 23 or 24, wherein said mammal is a cattle or human.

28. A method for producing an antibody specific to KefC (SEQ ID NO: 91); said method comprising the steps of
a) immunizing a mammal with said peptide or an immunogenic fragment thereof, and
b) purifying said antibody from the tissue of said mammal, or from a hybridoma made using said tissue.

29. The method of claim 28 wherein said antibody is a polyclonal antibody or fragment thereof.

30. The method of claim 28 wherein said antibody is a monoclonal antibody or fragment thereof.

31. A composition for stimulating an immune response against E. coli O157, said composition comprising substantially pure MopB (SEQ ID NO: 251) or an immunogenic fragment thereof.

32. The composition of claim 31, said composition further comprising a pharmaceutically acceptable carrier or an adjuvant.

33. A method of inducing an immune response to E. Coli O157 in a mammal, the method comprising administering to the mammal the composition of claim 31.

34. A method of treating a mammal for an E. Coli O157 associated disease, said method comprising administering the composition of claim 31.

35. The method of claim 34, wherein said treatment is a prophylactic.

36. The method of claim 34, wherein said treatment is therapeutic.

37. The method of claim 33 or 34, wherein said mammal is a cattle or human.

38. A method for producing an antibody specific to MopB (SEQ ID NO: 251); said method comprising the steps of
a) immunizing a mammal with said peptide or an immunogenic fragment thereof, and
b) purifying said antibody from the tissue of said mammal, or from a hybridoma made using said tissue.

39. The method of claim 38 wherein said antibody is a polyclonal antibody or fragment thereof.

40. The method of claim 38 wherein said antibody is a monoclonal antibody or fragment thereof.

41. A composition for stimulating an immune response against E. coli O157, said composition comprising substantially pure CysN (SEQ ID NO: 283) or an immunogenic fragment thereof.

42. The composition of claim 41, said composition further comprising a pharmaceutically acceptable carrier or an adjuvant.

43. A method of inducing an immune response to E. Coli O157 in a mammal, the method comprising administering to the mammal the composition of claim 41.

44. A method of treating a mammal for an E. Coli O157 associated disease, said method comprising administering the composition of claim 41.

45. The method of claim 44, wherein said treatment is a prophylactic.

46. The method of claim 44, wherein said treatment is therapeutic.

47. The method of claim 43 or 44, wherein said mammal is a cattle or human.

48. A method for producing an antibody specific to CysN (SEQ ID NO: 283); said method comprising the steps of
a) immunizing a mammal with said peptide or an immunogenic fragment thereof, and
b) purifying said antibody from the tissue of said mammal, or from a hybridoma made using said tissue.

49. The method of claim 48 wherein said antibody is a polyclonal antibody or fragment thereof.

50. The method of claim 48 wherein said antibody is a monoclonal antibody or fragment thereof.

51. A composition for stimulating an immune response against E. coli O157, said composition comprising substantially pure FolE (SEQ ID NO: 285) or an immunogenic fragment thereof.

52. The composition of claim 51, said composition further comprising a pharmaceutically acceptable carrier or an adjuvant.

53. A method of inducing an immune response to E. Coli O157 in a mammal, the method comprising administering to the mammal the composition of claim 51.

54. A method of treating a mammal for an E. Coli O157 associated disease, said method comprising administering the composition of claim 51.

55. The method of claim 54, wherein said treatment is a prophylactic.

56. The method of claim 54, wherein said treatment is therapeutic.

57. The method of claim 53 or 54, wherein said mammal is a cattle or human.

58. A method for producing an antibody specific to FolE (SEQ ID NO: 285); said method comprising the steps of
a) immunizing a mammal with said peptide or an immunogenic fragment thereof, and
b) purifying said antibody from the tissue of said mammal, or from a hybridoma made using said tissue.

59. The method of claim 58 wherein said antibody is a polyclonal antibody or fragment thereof.

60. The method of claim 58 wherein said antibody is a monoclonal antibody or fragment thereof.

61. A composition for stimulating an immune response against E. coli O157, said composition comprising substantially pure CirA (SEQ ID NO: 113) or an immunogenic fragment thereof.

62. The composition of claim 61, said composition further comprising a pharmaceutically acceptable carrier or an adjuvant.

63. A method of inducing an immune response to E. Coli O157 in a mammal, the method comprising administering to the mammal the composition of claim 61.

64. A method of treating a mammal for an E. Coli O157 associated disease, said method comprising administering the composition of claim 61.

65. The method of claim 64, wherein said treatment is a prophylactic.

66. The method of claim 64, wherein said treatment is therapeutic.

67. The method of claim 63 or 64, wherein said mammal is a cattle or human.

68. A method for producing an antibody specific to CirA (SEQ ID NO: 113); said method comprising the steps of
a) immunizing a mammal with said peptide or an immunogenic fragment thereof, and
b) purifying said antibody from the tissue of said mammal, or from a hybridoma made using said tissue.

69. The method of claim 68 wherein said antibody is a polyclonal antibody or fragment thereof.

70. The method of claim 68 wherein said antibody is a monoclonal antibody or fragment thereof.

71. A composition for stimulating an immune response against E. coli O157, said composition comprising substantially pure YeaF (SEQ ID NO: 175) or an immunogenic fragment thereof.

72. The composition of claim 71, said composition further comprising a pharmaceutically acceptable carrier or an adjuvant.

73. A method of inducing an immune response to E. Coli O157 in a mammal, the method comprising administering to the mammal the composition of claim 71.

74. A method of treating a mammal for an E. Coli O157 associated disease, said method comprising administering the composition of claim 71.

75. The method of claim 74, wherein said treatment is a prophylactic.

76. The method of claim 74, wherein said treatment is therapeutic.

77. The method of claim 73 or 74, wherein said mammal is a cattle or human.

78. A method for producing an antibody specific to YeaF (SEQ ID NO: 175); said method comprising the steps of
a) immunizing a mammal with said peptide or an immunogenic fragment thereof, and
b) purifying said antibody from the tissue of said mammal, or from a hybridoma made using said tissue.

79. The method of claim 78 wherein said antibody is a polyclonal antibody or fragment thereof.

80. The method of claim 78 wherein said antibody is a monoclonal antibody or fragment thereof.

81. A composition for stimulating an immune response against E. coli O157, said composition comprising substantially pure Z1931 (SEQ ID NO: 169) or an immunogenic fragment thereof.

82. The composition of claim 81, said composition further comprising a pharmaceutically acceptable carrier or an adjuvant.

83. A method of inducing an immune response to E. Coli O157 in a mammal, the method comprising administering to the mammal the composition of claim 81.

84. A method of treating a mammal for an E. Coli O157 associated disease, said method comprising administering the composition of claim 81.

85. The method of claim 84, wherein said treatment is a prophylactic.

86. The method of claim 84, wherein said treatment is therapeutic.

87. The method of claim 83 or 84, wherein said mammal is a cattle or human.

88. A method for producing an antibody specific to Z1931 (SEQ ID NO: 169); said method comprising the steps of
a) immunizing a mammal with said peptide or an immunogenic fragment thereof, and
b) purifying said antibody from the tissue of said mammal, or from a hybridoma made using said tissue.

89. The method of claim 88 wherein said antibody is a polyclonal antibody or fragment thereof.

90. The method of claim 88 wherein said antibody is a monoclonal antibody or fragment thereof.

91. A composition for stimulating an immune response against E. coli O157, said composition comprising substantially pure Z0269 (SEQ ID NO: 195) or an immunogenic fragment thereof.

92. The composition of claim 91, said composition further comprising a pharmaceutically acceptable carrier or an adjuvant.

93. A method of inducing an immune response to E. Coli O157 in a mammal, the method comprising administering to the mammal the composition of claim 91.

94. A method of treating a mammal for an E. Coli O157 associated disease, said method comprising administering the composition of claim 91.

95. The method of claim 94, wherein said treatment is a prophylactic.

96. The method of claim 94, wherein said treatment is therapeutic.

97. The method of claim 93 or 94, wherein said mammal is a cattle or human.

98. A method for producing an antibody specific to Z0269 (SEQ ID NO: 195); said method comprising the steps of
a) immunizing a mammal with said peptide or an immunogenic fragment thereof, and
b) purifying said antibody from the tissue of said mammal, or from a hybridoma made using said tissue.

99. The method of claim 98 wherein said antibody is a polyclonal antibody or fragment thereof.

100. The method of claim 98 wherein said antibody is a monoclonal antibody or fragment thereof.

101. A method of identifying a protein comprising the steps of:
(a) providing polyclonal antibodies, wherein said polyclonal antibodies are isolated from the sera of a subject infected with a pathogen,
(b) contacting said sera with a library of recombinant proteins under conditions allowing for complex formation between said polyclonal antibodies and said proteins, wherein said library is derived from said pathogen,
(c) identifying those proteins in said complex with said antibodies.

102. The method of claim 101, wherein said pathogen is E. coli O157.

103. The method of claim 101, wherein said polyclonal antibodies are coupled to a column.

104. The method of claim 101, wherein said proteins identified in step (c) are identified using GelC-MS/MS.

105. An isolated polypeptide selected from the group consisting of: (a) a polypeptide comprising an amino acid sequence having substantial identity to any one of the polypeptides identified in Table 2, Table 3, or Table 7; (b) a polypeptide comprising the amino acid sequence of any one of the polypeptides identified in Table 2, Table 3 or Table 7; (c) a polypeptide that consists essentially of any one of the polypeptides identified in Table 2, Table 3 or Table 7; and (d) a polypeptide comprising at least 200 contiguous amino acids of any one of the polypeptides identified in Table 2, Table 3 or Table 7.

106. An isolated nucleic acid molecule selected from the group consisting of: (a) a nucleic acid molecule having substantial identity to any one of the polynucleotides identified in Table 2 or Table 3; (b) a nucleic acid molecule comprising the nucleotide sequence of any one of the polynucleotides identified in Table 2 or Table 3 or a complement thereof; (c) a nucleic acid molecule consisting essentially of any one of the polynucleotides identified in Table 2 or Table 3 or a fragment thereof; and (d) a nucleic acid molecule comprising at least 20 contiguous nucleotides of any one of the polynucleotides identified in Table 2, Table 3 or Table 7

107. A vector or host cell comprising the isolated nucleic acid molecule of claim 106.

108. A method for screening a compound for effectiveness as an antagonist of a polypeptide of claim 1, the method comprising the steps of: a) exposing a sample comprising a polypeptide of claim 1 to a compound; and b) detecting antagonist activity in the sample, wherein said antagonist activity identifies said compound as an effective antagonist of a polypeptide of claim 105.

109. A composition comprising an antagonist compound identified by a method of claim 108.

110. A method for screening an antagonist compound for effectiveness in altering expression of a target nucleic acid molecule of claim 2, said method comprising the steps of: a) exposing a sample comprising a target nucleic acid molecule of claim 2 to a compound under conditions suitable for the expression of the target nucleic acid molecule; b) detecting altered expression of the target nucleic acid molecule; and c) comparing the expression of the target nucleic acid molecule in the presence of the compound and in the absence of the compound, wherein a decrease in expression identifies an antagonist compound effective in altering the expression of the target nucleic acid molecule of claim 106.

111. A composition comprising an antagonist compound identified according to the method of claim 110.

112. A method for the treatment of a subject having need to inhibit a polypeptide of claim 105, said method comprising administering to the individual a therapeutically effective amount of the antagonist of claim 5 or claim 7.

113. A vaccine composition comprising a pharmaceutically acceptable vehicle and an isolated immunogenic polypeptide of claim 105.

114. A method of preventing or treating a microbial infection in a mammal by administering to said mammal a vaccine composition of claim 113.

115. The method of claim 114, wherein said mammal is a human.
